SEQUENCE LISTING (36 sequences)

SEQ ID NO: 1 - DNA, 19 bp, artificial
  PCR primer
cgctggcggtactgaagtc

SEQ ID NO: 2 - DNA, 20 bp, artificial
  PCR primer
aaggaggaacggtgacatgc

SEQ ID NO: 3 - DNA, 22 bp, artificial
  PCR primer
aacctgaaccttccaaagatgg

SEQ ID NO: 4 - DNA, 21 bp, artificial
  PCR primer
tctggcttgttcctcactact

SEQ ID NO: 5 - DNA, 20 bp, artificial
  PCR primer
ccgtcgcccatcatcaagtt

SEQ ID NO: 6 - DNA, 20 bp, artificial
  PCR primer
ctgtctggggcagtccaaag

SEQ ID NO: 7 - DNA, 23 bp, artificial
  PCR primer
actgagagtgattgagagtggac

SEQ ID NO: 8 - DNA, 21 bp, artificial
  PCR primer
aaccctctgcacccagttttc

SEQ ID NO: 9 - DNA, 21 bp, artificial
  PCR primer
gcttgtccaagagtgcatggt

SEQ ID NO: 10 - DNA, 19 bp, artificial
  PCR primer
agggctggttctcgatggt

SEQ ID NO: 11 - DNA, 19 bp, artificial
  PCR primer
gcctctcgtcaggcttgag

SEQ ID NO: 12 - DNA, 22 bp, artificial
  PCR primer
tcatctaggtcaactcgtgcaa

SEQ ID NO: 13 - DNA, 20 bp, artificial
  PCR primer
gcagattagtaggcggcttg

SEQ ID NO: 14 - DNA, 20 bp, artificial
  PCR primer
gctggtggaacaattcctgt

SEQ ID NO: 15 - DNA, 20 bp, artificial
  PCR primer
ggccctcgtgctgatgctac

SEQ ID NO: 16 - DNA, 20 bp, artificial
  PCR primer
tggagcagcagcagctccgc

SEQ ID NO: 17 - DNA, 20 bp, artificial
  PCR primer
ggactagtggagaaggtgcg

SEQ ID NO: 18 - DNA, 20 bp, artificial
  PCR primer
gggcgctgcccatcatcatg

SEQ ID NO: 19 - DNA, 20 bp, artificial
  PCR primer
caccgaatagttacggtcgg

SEQ ID NO: 20 - DNA, 20 bp, artificial
  PCR primer
gcacgggtcgggtgagagtg

SEQ ID NO: 21 - DNA, 20 bp, artificial
  PCR primer
ggggacctagagcaacttac

SEQ ID NO: 22 - DNA, 20 bp, artificial
  PCR primer
gtagcagtctcctggcaatc

SEQ ID NO: 23 - DNA, 20 bp, artificial
  PCR primer
ctcaaccgcttcggcaagac

SEQ ID NO: 24 - DNA, 20 bp, artificial
  PCR primer
ggactggtaccggaggtgtc

SEQ ID NO: 25 - DNA, 20 bp, artificial
  PCR primer
gagtcaacggatttggtcgt

SEQ ID NO: 26 - DNA, 20 bp, artificial
  PCR primer
ttgattttggagggatctcg

SEQ ID NO: 27 - DNA, 39 bp, artificial
  PCR primer
cggtttgtttgggtttgggtttgggtttgggtttgggtt

SEQ ID NO: 28 - DNA, 38 bp, artificial
  PCR primer
ggcttgccttacccttacccttacccttacccttaccc

SEQ ID NO: 29 - DNA, 24 bp, Homo sapiens
tgacttttggactttgtttcttta

SEQ ID NO: 30 - DNA, 22 bp, Homo sapiens
tgacttttggactttgtcttta

SEQ ID NO: 31 - DNA, 18 bp, Homo sapiens
ctgtcctgggattctctt

SEQ ID NO: 32 - DNA, 18 bp, Homo sapiens
ctgcctggggattctctt

SEQ ID NO: 33 - DNA, 24 bp, Homo sapiens
ttggaagaaaataatcaagaagag

SEQ ID NO: 34 - DNA, 20 bp, Homo sapiens
ttggaagaaaataagaacag

SEQ ID NO: 35 - DNA, 14 bp, Homo sapiens
atgaagaaagagga

SEQ ID NO: 36 - DNA, 14 bp, Homo sapiens
atgagaaagaggaa

(19) United States
(12) Patent Application Publication
(10) Pub. No.: US 2013/0309249 A1
(43) Pub. Date: Nov. 21, 2013

(54) MATERIALS AND METHODS FOR THE PROPHYLACTIC TREATMENT OF A PRE-MALIGNANT CONDITION

(71) Applicants: Charlotte Kuperwasser, Boston, MA (US); Maja Sedic, Cambridge, MA (US)

(72) Inventors: Charlotte Kuperwasser, Boston, MA (US); Maja Sedic, Cambridge, MA (US)

(21) Appl. No.: 13/869,796

(22) Filed: Apr. 24, 2013

Related U.S. Application Data
(60) Provisional application No. 61/637,578, filed on Apr. 24, 2012.

(51) Int. Cl. A61K 39/395; A61K 31/352; A61K 38/50; A61K 31/05; A61K 38/45; A61K 31/12; A61K 31/455

(52) U.S. Cl. CPC A61K 39/3955; A61K 31/12; A61K 31/352; A61K 31/455; A61K 31/05; A61K 38/45; A61K 38/50
USPC 424/172.1; 514/685; 514/456; 514/355; 514/733; 424/94.5; 424/94.6

(57) ABSTRACT

Described herein are materials and methods for the prophylactic treatment of a pre-malignant condition, comprising administering a SIRT1 agonist to an individual whose genotype comprises one defective BRCA1 allele and one functional BRCA1 allele.

### RELATED APPLICATION

This application claims the benefit of U.S. provisional application No. 61/637,578, filed Apr. 24, 2012, the entire contents of each of which are herein incorporated by reference.

### BACKGROUND

Individuals with inherited mutations in BRCA1 have a ˜50-85% chance of developing breast and/or ovarian cancer within their lifetimes. Although BRCA1 function appears to be essential in all cell types, it is unclear why increased risk of cancer development in individuals with mutations in BRCA1 is restricted to only a select few tissues. The precise molecular basis for this cell and tissue-type specific predisposition and accelerated tumor development is unknown.

Although BRCA1 function appears to be essential in all cell types, increased risk of cancer development in individuals with mutations in BRCA1 is restricted to specific tissues; moreover, BRCA1-associated breast cancers invariably develop at an early age with a rapid onset. Unfortunately, non-invasive forms of preventative therapies are not available for individuals with germline mutations in BRCA1. Surgical removal of all breast tissue is the only approved method for preventing disease in these patients, which is a major and irreversible treatment that carries considerable negative emotional and physical side effects. In addition, although prophylactic mastectomies reduce the risk of developing breast cancer by −90%, some women still develop breast cancer from the residual breast epithelium left behind.

Accordingly, there is a need to determine the pathway that leads from particular BRCA1 genotypes to malignancy, as such a pathway would be informative for not only breast cancer, but other cancers as well that share downstream effectors.

### SUMMARY

Mammary epithelial cells (HMECs), but not other cell types, from individuals harboring a deleterious allele of BRCA1 (BRCA1^{mut/+}) undergo rapid telomere attrition and premature senescence in the absence of tumor suppressor loss. Disclosed herein are findings that haploinsufficiency-induced senescence (HIS), genomic instability and other pre-malignant events in BRCA1 heterozygotes are due to the misregulation of the NAD⁺-dependent deacetylase, SIRT1 (the yeast Sir2 homolog) in HMECs, but not fibroblasts. SIRT1 misregulation leads to increased acetylyation of Rb, telomere dysfunction and senescence. HIS, genomic instability and other pre-malignant events in BRCA1^{mut/+} HMECs can be, therefore, prevented and/or prevented from progressing further, through the activation of SIRT1.

This work provides a novel paradigm for the prophylactic treatment of high risk patients. This deeper understanding of the molecular mechanisms affected by BRCA1 haploinsufficiency has significant and far-ranging implications for the basis by which mutation in BRCA1 preferentially leads to the rapid onset and tissue-specificity of BRCA1-associated cancers. Data provided herein indicate drugs targeting SIRT1 activity offer novel prophylactic and/or ameliorative therapies for individuals with mutations in BRCA1.

One embodiment is directed to a method for prophylactically treating an individual at risk for breast cancer or ovarian cancer, comprising identifying the individual as comprising a genotype that comprises one copy of a defective BRCA1 allele and one copy of a functional BRCA1 allele, and administering a prophylactically effective amount of a SIRT1 agonist to the individual. In a particular embodiment, the prophylactic treatment prevents or ameliorates a pre-malignant condition associated with breast cancer or ovarian cancer. In a particular embodiment, the SIRT1 agonist is selected from the group consisting of: a small molecule agonist, an activating antibody and an enzymatic agonist. In a particular embodiment, the SIRT1 agonist is selected from the group consisting of: butein, fisetin, isonicotinamide, piceatannol, quercetin and resveratrol.

One embodiment is directed to a method for prophylactically treating an individual at risk for breast cancer or ovarian cancer, comprising identifying the individual as comprising a genotype that comprises one copy of a defective BRCA1 allele and one copy of a functional BRCA1 allele, and administering a prophylactically effective amount of a deacetylase that deacetylates Rb. In a particular embodiment, the method further comprises administering a prophylactically effective amount of a Rb phosphorylase.

### BRIEF DESCRIPTION OF THE DRAWINGS

FIGS. 1A-1C are data showing ex vivo growth characteristics of HMECs. FIG. 1A is a representative growth curve of WT HMECs grown in culture. FIG. 1B shows Western blot analysis of p16/INK4a, p53, phosphorylated p53 and p21 as cells enter and are at M0, emerge from M0 and enter agonescence. FIG. 1C shows representative images of BRCA1 WT HMECs taken at different growth phases.

FIGS. 2A-2D are data and images showing BRCA1^{mut/+} HMECs, but not fibroblasts, undergo premature senescence. FIG. 2A shows representative growth curves of WT and BRCA1^{mut/+} HMECs and fibroblasts and HMECs in culture. Each curve represents an average of BRCA1^{+/+} (n=3) and BRCA1^{mut/+} (n=3) patient samples. FIG. 2B shows SA-β-galactosidase staining of cells at indicated points. Point A=hyperproliferating growth phase, Point B=senescence. FIG. 2C shows graphs quantifying SA-βgal positive cells at indicated time points. FIG. 2D shows Western blot analysis of p16/INK4a, p53, phosphorylated p53, and p21 as WT and BRCA1^{mut/+} HMECs enter and are at M0 or agonescence.

FIGS. 3A-3E show data for DNA damage and DDR analysis in BRCA1^{mut/+} HMECs and fibroblasts. FIG. 3A shows telomere length is similar between pre-senescent WT and BRCA1^{mut/+} HMECs. FIG. 3B shows total chromosomal abnormalities events were similar between WT and BRCA1^{mut/+} HMECs, although differences in types of rearrangements were noted. FIG. 3C shows Western blot analysis indicating the DDR hallmarks of senescence are not engaged in growth arrested BRCA1^{mut/+} HMECs. Western blot analysis and quantification of p53, phospho-p53, and phosphorylated γH2AX in WT and BRCA1^{mut} HMECs was performed during the hyperproliferation phase (A) and during senescence (B). FIG. 3D shows IF and quantification of γH2AX foci in WT and BRCA1^{mut} during senescence. FIG. 3E shows expression and quantification of total and phosphorylated p53 protein in senescent WT and BRCA1^{mut} fibroblasts (N=3 patients each genotype).

FIG. 4 shows loss of heterozygosity (LOH) analysis indicating no BRCA1 LOH events in senescent BRCA1^{mut/+} HMECs. gDNA from proliferating and senescent BRCA1^{mut/+} HMECs was sequenced over the mutation site of BRCA1 to determine whether heterozygosity was lost. All BRCA1^{mut/+} HMECs remained heterozygous at senescence (three different patient samples; see FIGS. 1-3).

FIGS. 5A-5C show data indicating the molecular hallmarks of senescence are differentially engaged in senescent BRCA1^{mut/+} HMECs. FIG. 5A shows expression of p53 pathway-related genes involved in the processes of apoptosis, cell cycle, cell growth, proliferation and DNA repair in senescent BRCA1^{mut/+}. FIG. 5B shows Western blot analysis of Rb and p53 pathway components in senescent WT and BRCA1′ HMECs and in senescent WT HMECs following knockdown of BRCA1 expression. FIG. 5C shows quantification of p53 and pRB pathways components in growing or senescent WT and BRCA1^{mut/+} fibroblasts (N=3) patients or (D) HMECs (N=2 patients).

FIGS. 6A-6E show data indicating SIRT1 levels are reduced in BRCA1^{mut/+} HMECs, but not fibroblasts, and the loss of SIRT1 activity leads to premature senescence. FIG. 6A shows Western blot analysis of SIRT1 in exponentially growing WT and BRCA^{mut} HMECs (A) as well as senescent WT and BRCA1^{mut} HMECs. Western blot analysis of SIRT1 in WT HMECs in which BRCA1 was knocked down using shRNA. FIG. 6B shows quantification of protein levels in HMECs (N=2 patients), fibroblasts (N=3 patients), and shBRCA1 in WT HMECs. FIG. 6C shows Western blot analysis of SIRT1 in WT HMECs in which SIRT1 was knocked down using shRNA. FIG. 6D shows mRNA analysis of SIRT1 in WT HMECs in which SIRT1 was knocked down using shRNA. FIG. 6E shows SA-β-galactosidase staining of HMECs following infection of either control (shScr) or 2 different hairpins targeting SIRT1 expression.

FIGS. 7A-7C shows data obtained during cultured growth of BRCA^{+/+} and BRCA^{mut/+} HMECs. FIG. 7A shows representative growth curves of BRCA1^{+/+} and BRCA1^{mut/+} HMECs in culture. BRCA1^{mut/+} HMECs undergo premature growth arrest with significantly increased number of β-galactosidase positive cells as compared to WT. FIG. 7B shows Western blot analysis indicating phospho-Rb and cyclin A levels are decreased. FIG. 7C shows the expression of stress activated serum factors (SASFs) is increased in senescent BRCA1^{mut/+} HMECs in comparison to agonescent BRCA1 WT HMECs.

FIGS. 8A and 8B show the protein expression levels of various activators and controls in BRCA^{+/+} and BRCA^{mut/+} HMECs. FIG. 8A shows Western blot analysis of total p53 and γH2AX levels are decreased in senescent BRCA1^{mut/+} HMECs in comparison to agonescent BRCA1 WT HMECs (see boxes). FIG. 8B shows a decrease in γH2AX levels in senescent BRCA1^{mut/+} HMECs (also confirmed by immunofluorescence, right panel).

FIGS. 9A and 9B show genomic abnormality events in proliferating HMECs. FIG. 9A is a metaphase spread of proliferating BRCA1 WT and BRCA1^{mut/+} HMECs. FIG. 9B is a table showing the percent of total chromosomal abnormalities is increased in BRCA1^{mut/+} HMECs. Increase in particular types of chromosomal abnormalities such as, for example, trisomy and aneuploidy are only seen in BRCA1^{mut/+} HMECs.

FIGS. 10A-10F show BRCA1^{mut/+} HMECs exhibit increased genomic instability and telomere dysfunction. FIG. 10A: representative images of IF staining for phospho-ATM/ATR substrates, γH2AX foci, 53BP1 foci in proliferating (Prolif) WT and BRCA1^{mut/+} HMECs. Graphs under the respective images represent the percent of cells positive for phospho-ATM/ATR substrates, as well as the average number of γH2AX foci per WT nuclei (N=278) and BRCA1 nuclei (N=182), and the average number of 53BP1 foci per WT nuclei (N=262) and BRCA1 nuclei (N=187). FIG. 10B: representative images and summary table of significant genetic and chromosomal events determined by karyotype analysis in proliferating WT and BRCA1^{mut/+} HMECs. FIG. 10C: representative images of IF staining for γH2AX foci in WT and BRCA1 mut/+HMFs at day 100 in culture (same day in culture as proliferating HMECs). Graph under the images represents the average number of γH2AX foci per WT nuclei (N=278) and BRCA1 nuclei (N=182). Representative images and summary table of significant genetic and chromosomal events determined by karyotype analysis in WT and BRCA1^{mut/+} HMFs at day 100 in culture (same day in culture as proliferating HMECs). FIG. 10D: telomere erosion rate in WT (N=4) and BRCA1^{mut/+} (N=4) HMECs and keratinocytes (HDE, WT N=3, BRCA1^{mut/+} N=3). Telomere length was determined by qPCR in proliferating and agonescent/senescent WT and BRCA1^{mut/+} HMECs. Telomere erosion rate was calculated by the following formula: TER=(M2/M* telomere length-Prolif. telomere length)/PDs. FIG. 10E: average telomere length in hTERT immortalized WT (N=3) and BRCA1^{mut/+} (N=3) HMECs. FIG. 10F: representative images and summary table of significant genetic and chromosomal events determined by karyotype analysis in immortalized WT and BRCA1^{mut/+} HMECs. Scale bar=10 μm.

FIGS. 11A-11H show premature growth arrest in cells from BRCA1 mutation carriers is cell type-specific. FIG. 11A: representative growth curves of WT (N=3) and BRCA1^{mut/+} (N=3) HMECs, HDEs and HMFs. FIG. 11B: Western blot analysis of p161NK4a, total p53, p53 (Ser15) and p21 levels in WT and BRCA1^{mut/+} HMECs at indicated population doublings (PDs). Selected population doublings correspond to the following growth stages: M0=Stasis, M2=Agonescence (WT HMECs), M*=Premature growth arrest (BRCA1 HMECs). FIG. 11C: quantification of Ki67-positive cells by immunofluorescence (IF) staining in M2 WT and M* BRCA1^{mut/+} HMECs. FIG. 11D: quantification of Trypan blue-positive cells in M2 WT and M* BRCA1^{mut/+} HMECs. FIG. 11E: brightfield images of SA-β-galactosidase staining and quantification of positive cells at selected PDs in WT and BRCA1^{mut/+} HMECs and HMFs. FIG. 11F: Western blot analysis of p161NK4a, total p53, p53 (Ser15) and p21 levels in WT and BRCA1^{mut/+} HMFs at indicated population doublings (PDs). M1=Senescence. FIG. 11G: Western blot analysis of p16INK4a, total p53, p53 (Ser15) and p21 levels in WT and BRCA1^{mut/+} HDEs at indicated population doublings (PDs). M0=Stasis. FIG. 11H: LOH analysis in proliferating (Prolif) and cell cycle-arrested (M*) BRCA1^{mut/+} HMECs and HDEs. Scale bar=100 μm.

FIGS. 12A-12G show a characterization of BRCA1 haploinsufficiency-induced senescence. FIG. 12A: Western blot analysis of p53 (Ser15), total p53, γH2AX, p21 and p27 levels in proliferating (Prolif) and agonescent/growth-arrested (M2/HIS) WT and BRCA1^{mut/+} HMECs and proliferating (Prolif) and M0 WT and BRCA1^{mut/+} HDEs. Quantification of p53 (Ser15), total p53, γH2AX, p21 and p27 levels determined by western blotting in M2 WT (N=3) and HIS BRCA1^{mut/+} (N=3) HMECs and M0 WT (N=3) and HIS BRCA1^{mut/+} (N=3) HDEs is represented in the graphs below images. FIG. 12B: representative images of IF staining for phospho-ATM/ATR substrates, γH2AX foci, 53BP1 foci in M2 WT and MHIS BRCA1^{mut/+} HMECs. Graphs under the respective images represent the percent of cells positive for phospho-ATM/ATR substrates, and the average number of γH2AX foci per WT nuclei (N=51) and BRCA1 nuclei (N=78), as well as the number of 53BP1 foci per WT nuclei (N=79) and BRCA1 nuclei (N=99). FIG. 12C: Western blot analysis of phospho-pRb (Ser795), total pRb, Cyclin E and Cyclin A levels in proliferating (Prolif) and agonescent/growth-arrested (M2/HIS) WT and BRCA1^{mut/+} HMECs and proliferating (Prolif) and M0 WT and BRCA1^{mut/+} HDEs. Quantification of phospho-pRb (Ser795), total pRb, Cyclin E and Cyclin A levels determined by western blotting in M2 WT (N=3) and HIS BRCA1^{mut/+} (N=3) HMECs and M0 WT (N=3) and HIS BRCA1^{mut/+} (N=3) HDEs is represented in the graphs below images. FIG. 12D: mRNA levels of IL-6 and MMP2 (SASFs) in M2 WT and M* BRCA1^{mut/+} HMECs. The values were determined by qRT-PCR and normalized to proliferating cells (represented by line set at 1). FIG. 12E: Rb knockdown in proliferating BRCA1^{mut/+} HMECs (mRNA and protein levels shown). Representative growth curve of control (shScr) and shRb BRCA1^{mut/+} HMECs. FIG. 12F: Western blot analysis of p53 (Ser15), total p53, p21 and p27 levels in growth arrested shScr (control) and shRb BRCA1^{mut/+} HMECs. FIG. 12G: representative images and summary table of significant genetic and chromosomal events determined by karyotype analysis in proliferating shScr (control) and shRb BRCA1^{mut/+} HMECs.

FIGS. 13A-13H demonstrate, in BRCA1^{mut/+} HMECs, SIRT1 regulates HIS through acetylation of pRb and histone H4K16. FIG. 13A: Western blot analysis and quantification of SIRT1 levels in proliferating (Prolif) and growth arrested (M2/MHIS) WT (N=3) and BRCA1^{mut/+} (N=3) HMECs, proliferating (Prolif) and M0 WT and BRCA1^{mut/+} HDEs, as well as proliferating (Prolif) and senescent (M1) WT (N=3) and BRCA1^{mut/+} (N=3) HMFs and HDFs. FIG. 13b: i). Acetyl-pRb (Acetyl-Lysine blot) and total pRb levels in shScr (control), shSIRT1 and shBRCA1 HMECs. Total pRb was immunoprecipitated (IP), and the Western blot was sequentially probed with anti-Acetyl-Lysine as well as anti-pRb (total) antibodies. ii) Western blot analysis of Acetyl-Lysine, total-pRb and tubulin (loading control) levels in total cell lysates from shScr (control), shSIRT1 and shBRCA1 HMECs. FIG. 13C: SIRT1 levels (protein and mRNA) in shScr (control), shSIRT1-1, and shSIRT1-2 HMECs. Analysis of SA-β-galactosidase staining in HMECs with shScr (control) or shSIRT1-1. FIG. 13D: ChIP analysis of SIRT1 abundance at telomeres in WT and BRCA1 mutation carriers HMECs. Data are presented as average of 3 WT and 3 BRCA1 patient samples±SEM. FIG. 13E: Western blot analysis of Acetyl-H3K9, total H3, Acetyl-H4K16 and total H4 levels in shScr (control), shSIRT1 and shBRCA1 HMECs from Patient 1 and Patient 2. FIG. 13F: ChIP analysis of Acetyl-H4K16 and Acetyl-H3K9 abundance at telomeres in shScr (control), shSIRT1 and shBRCA1 HMECs from Patient 1 and Patient 2 (results were averaged). FIG. 13G: representative images and mean telomere length (kb) determined by qFISH in WT lobules (N=762 cells) and BRCA1^{mut/+} lobules (N=800 cells). FIG. 13H: images of immunohistochemistry (1HC) staining and quantification of SIRT1 levels in epithelial cells from WT (N=10) and BRCA1^{mut/+} (N=10) breast tissues. Allred score methodology was used to measure SIRT1 antibody staining.

FIGS. 14A-14D show further cell analysis. FIG. 14A: representative growth curve of WT HMECs. FIG. 14B: Western blot analysis of p16INK4a, total p53, p53 (Ser15) and p21 levels in WT and BRCA1^{mut/+} HMECs at indicated population doublings (PDs). Selected population doublings correspond to the following growth stages: M0=Stasis (HMECs), M2=Agonescence (WT HMECs). FIG. 14C: representative phase-contrast brightfield images of WT HMECs in M0, post-M0 proliferation and M2 growth stages. FIG. 14D: images of Ki67-positive cells by immunofluorescence (IF) staining in M2 WT and M* BRCA1^{mut/+} HMECs.

FIGS. 15A-15D show further cell analysis. FIG. 14A: Western blot analysis of p161NK4a, total p53, p53 (Ser15) and p21 levels in Patient 2 and Patient 3 of WT and BRCA1^{mut/+} HMFs at indicated PDs. FIG. 15B: representative growth curves of WT (N=3) and BRCA1^{mut/+} (N=3) HDFs. Analysis of SA-β-galactosidase levels using β-galactosidase detection assay in senescent WT and BRCA1^{mut/+} HDFs. FIG. 15C: Western blot analysis of p16INK4a, total p53, p53 (Ser15) and p21 levels in proliferating and senescent (M1) WT (N=3) and BRCA1^{mut/+} (N=3) HDFs.

FIGS. 16A-16F show further cell analysis. FIG. 16A: Western blot analysis of p53 (Ser15), total p53, γH2AX, p21 and p27 levels in proliferating (Prolif) and agonescent/senescent (M2/HIS) WT and BRCA1^{mut/+} HMECs from Patient 2 and Patient 3. FIG. 16B: Western blot analysis of phospho-pRb (Ser795), total pRb, Cyclin E and Cyclin A levels in proliferating (Prolif) and agonescent/growth-arrested (M2/M*) WT and BRCA1^{mut/+} HMECs from Patient 2 and Patient 3. FIG. 16C: mRNA levels of Cyclin A in M2 WT and M* BRCA1^{mut/+} HMECs. The values were determined by qRT-PCR and normalized to proliferating cells (represented by line set at 1). FIG. 16D: mRNA levels of p14, p15, p18 and p19 in M2 WT and HIS BRCA1^{mut/+} HMECs. The values were determined by qRT-PCR and normalized to proliferating cells (represented by line set at 1). FIG. 16E: mRNA levels of IL-8 and PAI-1 (SASFs) in M2 WT and M* BRCA1^{mut/+} HMECs. The values were determined by qRT-PCR and normalized to proliferating cells (represented by line set at 1). FIG. 16F: i) pRb knockdown in proliferating BRCA1^{mut/+} HMECs from Patient 2 (mRNA levels). ii) Growth curve of shScr (control) and shpRb BRCA1^{mut/+} HMECs from Patient 2.

FIGS. 17A-17D show further cell analysis. FIG. 17A: Western blot analysis of p53 (Ser15), total p53, γH2AX, p21, p27 and p16INK4a levels in proliferating (Prolif) and growth-arrested (M0/HIS) WT and BRCA1^{mut/+} HDEs in additional patient samples. FIG. 17B: quantification of p53 (Ser15), total p53, γH2AX, p21 and p27, γH2AX, p27 and p16 levels determined by western blotting in M2 WT (N=3) and HIS in BRCA1^{mut/+} (N=3) HDEs. FIG. 17C: Western blot analysis of phospho-pRb (Ser795), total pRb, and Cyclin A levels in proliferating (Prolif) and growth-arrested (M0/HIS) WT and BRCA1^{mut/+} HDEs in additional patient samples. FIG. 17D: model of tissue and cell type-specific response to BRCA1-haploinsufficiency.

FIGS. 18A-18E show further cell analysis. FIG. 18A: Western blot analysis of SIRT1 levels in additional patient samples from proliferating (Prolif) and M2/MHIS WT and BRCA1^{mut/+} HMECs. Western blot analysis of SIRT1 levels in proliferating (Prolif) and M0 WT and BRCA1^{mut/+} HDEs from Patient 2 and Patient 3. FIG. 18B: Western blot analysis of SIRT1 levels in proliferating (Prolif) and M1 WT and BRCA1^{mut/+} HMF and HDFs from Patient 2 and Patient 3. FIG. 18C: Western blot analysis of SIRT1 levels in shLuciferase (control) and shBRCA1 HMECs. FIG. 18D: Analysis of SA-β-galactosidase staining using β-galactosidase detection assay in HMECs using a second short hairpin targeting SIRT1 (shSIRT1-2). FIG. 18E: median telomere length (kb) determined by qFISH in stromal fibroblasts from WT (N=21) and BRCA1^{mut/+} (N=10) disease-free patient tissues.

FIGS. 19A and 19B show pathway analysis of mammary epithelial cells in vivo from BRCA1-mutation carriers. FIG. 19A: Ingenuity Pathway Analysis identified 25 significant gene networks from differentially expressed genes from freshly isolated HMECs. A gene network of 12 overlapping central nodes was constructed from this analysis. Genes colored in grey represent genes differentially expressed in BRCA1^{mut/+} tissues vs. WT tissues. Nodes are displayed using various shapes that represent the functional class of the gene product. Edges with dashed lines show indirect interaction, while a continuous line represents direct interactions. FIG. 19B: the most significant gene networks identified involved central nodes in SIRT, Rb, p53 and NFκB pathways.

### DETAILED DESCRIPTION

Described herein are materials and methods for the prophylactic treatment of individuals at risk for cancer or a pre-malignant condition. Data provided herein elucidate the molecular and cellular basis for the cell type-specific predisposition and accelerated rate of tumor progression in BRCA1-mutation carriers and identify mechanisms that can be targeted for the prevention of cancer (e.g., breast cancer and ovarian cancer) and/or for the prevention of the progression of a pre-malignant condition.

Mutations in BRCA1 predispose in part, to the formation of aggressive human breast cancers in humans (Proia, T. et al., Cell Stem Cell, 8:149-63, 2011). Breast epithelial cells from BRCA1-mutation carries exhibit perturbations in differentiation programs prior to the formation of cancer. These defects are sufficient to disrupt the lineage commitment programs and influence tumor phenotype following neoplastic transformation. These findings along with those of others (Keller, P. et al., Proc. Natl. Acad. Sci. USA, 109:2772-7, 2011; Jeselsohn, R. et al., Cancer Cell, 17:65-76, 2010) provide molecular evidence supporting the notion that BRCA1 in human breast epithelial cells is associated with features of malignancy and a pre-malignant condition.

Described herein is the unexpected finding that haploinsufficiency for BRCA1 leads to defects in breast epithelial differentiation and lineage commitment, and a premature senescent state. Although several mouse models of BRCA1-deficiency exist, they have been unable to recapitulate many of the features of BRCA1 mutation in humans, including defects in mammary differentiation or increased frequency of tumor formation (Liu, X. et al., Proc. Natl. Acad. Sci. USA, 104:12111-6, 2007; Drost, R. & Jonkers, J., Br. J. Cancer, 101:1651-7, 2009; Xu, X. et al., Nat. Genet., 22:37-43, 1999; Xu, X. et al., Nat. Genet., 28:266-71, 2001; Moynahan, M., Oncogene, 21:8994-9007, 2002). BRCA1 heterozygous mice, in fact, do not exhibit any apparent abnormal phenotype, nor do they develop spontaneous mammary tumors. Conditional deletion of BRCA1 in mouse mammary epithelial cells, furthermore, does not result in accelerated tumor formation; rather, these mice develop mammary tumors at a low frequency and late in life. Only in the background of additional genetic mutations, such as heterozygosity for p53, is mammary tumorigenesis observed. Given the differences in p53, BRCA1 and telomere biology between mice and humans (Rangarajan, A. & Weinberg, R., Nat. Rev. Cancer, 3:952-9, 2003), studying BRCA1 haploinsufficiency in mice is not a legitimate approach to enumerate the phenotypes and mechanisms associated with BRCA1 haploinsufficiency in humans.

Described herein, for example, are observations using cells from human BRCA1-mutation carriers in various stages of growth including, for example, cellular senescence and immortalization. The use of these cells allowed for the identification of mechanisms of the regulation of a novel proliferative barrier in BRCA1^{mut/+} cells that is cell type-specific, namely, a SIRT1-dependent pathway that is involved in cell type-specific senescence and genomic instability. These findings contribute to the understanding of the molecular underpinnings and events that are necessary to drive cancer in BRCA1-mutation carriers; and provide a novel paradigm for the prophylactic treatment of high risk patients.

Tumor suppressor genes, such as BRCA1, repress malignant transformation by ensuring the fidelity of DNA replication and chromosomal segregation in response to potentially deleterious events. It had been assumed that individuals with inherited mutations in BRCA1 are predisposed to breast and ovarian cancer due to compromised DNA damage repair. Since this proposed function of BRCA1 is essential for all cell-lineages, it was puzzling as to why BRCA1-mutations are preferentially associated with increased incidence in a select few tissues rather than a generalized increase in all cancer types (as is observed with p53 and ATM).

Using human mammary epithelial cells (HMECs) and fibroblasts (HMFs) isolated from disease-free age-matched BRCA1-mutation carriers (BRCA1^{mut/+}) and non-carriers (BRCA1^{+/+}), disclosed herein is the finding that BRCA1^{mut/+} HMECs, but not fibroblasts, undergo premature senescence. This phenotype correlates with active Rb signaling pathway and reduced levels of SIRT1, but is not associated with increased activation of DNA damage response or a loss of heterozygosity (LOH) event.

As used herein, the singular forms “a,” “an” and “the” include plural references unless the content clearly dictates otherwise. Thus, for example, reference to “an agonist” includes a mixture of two or more such agonists.

As used herein, “treatment” refers to the administration of an agent to an individual, for either prophylaxis (“prophylactic treatment”) or to cure or reduce the symptoms of the infirmity or malady in the instance where the patient is afflicted. The methods and compounds described herein or identified through methods described herein can be used as part of a treatment regimen in therapeutically effective amounts or prophylactically effective amounts as would be determined by one of skill in the art. A “prophylactically effective amount” is an amount sufficient to decrease, prevent or ameliorate symptoms or conditions associated with a medical condition, e.g., a pre-malignant condition. The present disclosure, for example, is directed to treatment using a prophylactically effective amount of a compound sufficient to prevent or reduce the risk for developing a pre-malignant condition and/or to reduce or prevent further progression of a pre-malignant condition.

As used herein, the terms “individual,” “subject,” “host” and “patient” are used interchangeably and refer to any subject for whom diagnosis, treatment or therapy is desired, particularly humans. Other individuals include, but are not limited to cattle, dogs, cats, guinea pigs, rabbits, rats, mice, horses, and the like.

As used herein, a “pre-malignant condition” is a condition that, if left untreated, could lead to cancer. A pre-malignant condition is characterized by pre-malignant events, such as, for example, genomic instability (e.g., telomere shortening, aberrant centromere replication), escape from cell cycle arrest, escape from apoptosis, lesions etc.

Described herein for the first time is a link between BRCA1 haploinsufficiency and reduced activity levels of SIRT1. Although indications have been noted that SIRT1 levels are reduced in BRCA1-associated tumors (Wang, R. et al., Mol. Cell, 32:11-20, 2008), it is herein shown that the heterozygous carriers of a deleterious BRCA1 allele lead to the tissue-specific BRCA1 tumors. While not wishing to be bound by theory, reduced SIRT1 activity leads to both cell cycle arrest (and haploinsufficiency-induced senescence (HIS)), and genomic instability. The increase in genomic stability caused by reduced SIRT1 activity eventually results in escape from cell cycle arrest and apoptosis with a high level of genomic rearrangements due to increased genomic instability caused by reduced SIRT1 activity. As the mechanism leading to HIS would not be expected to lead to progression from a pre-malignant state, as it would appear to be a tumor repression event, the finding that reduced SIRT1 activity leads to both HIS and progression from a pre-malignant condition is entirely unexpected. It was this finding that allows one of skill in the art a novel prophylactic treatment for individuals who have a genetic predisposition to breast and ovarian cancer (e.g., individuals who are heterozygous for BRCA1)-namely, prophylactic treatment using a SIRT1 agonist.

As used herein, the term “agonist” refers to a compound or molecule that increases the biological activity of a second compound or molecule. A “SIRT1” agonist, for example, increases the biological activity of SIRT1. An agonist can stimulate the enzymatic activity of the SIRT1 protein, or it can lead to increased expression of SIRT1 through increased transcription and/or translation. Examples of SIRT1 agonist are known and can include, for example, small molecules, activating antibodies, proteins (e.g., enzymatic co-activators), and upstream effector signals. An agonist can either activate SIRT1 activity directly or indirectly through other effector molecules. Examples of SIRT1 agonists, sometimes referred to as sirtuin-activating compounds (STACs), include, for example, resveratrol, butein, fisetin, isonicotinamide (IsoNAM), piceatannol, quercetin, as well as compounds identified in Table 3 of US Patent Publication No: 2011/0152254, which is herein incorporated by reference in its entirety (see also, Milne, J. et al., Nature, 450:712-6, 2007, the entire contents of which are herein incorporated by reference) and/or as described (US Patent Publication Nos: 2012/0022254, 2011/0306612, 2011/0263564, 2011/0257174, 2011/0130387, 2011/0039847, 2011/0015192, 2010/0215632, 2009/0221020, 2009/0163476, 2009/0105246, 2009/0099170, 2009/0069301, 2009/0012080, 2008/0293081, and 2008/0249103, the entire contents of each of which are hereby incorporated by reference). SIRT1 mRNA levels were shown to increase in adipose tissue with a sixteen-week treatment of a combination of ephedrine, caffeine and the anti-diabetic drug Pioglitazone in non-diabetic human subjects (Bogacka, I. et al., Diabetes Care, 30:1179-86, 2007). Dietary supplementation of omega-3 fatty acids is effective in reversing the reduction of SIRT1 levels in rats with mild traumatic brain injury (Wu, A. et al., J. Neurotrauma, 24:1587-95, 2007).

In addition to known SIRT1 agonists, described herein are methods for identifying additional SIRT1 agonists useful for the methods described herein. Ideally, a SIRT1 agonist for use in the treatment methods described herein is effective in increasing SIRT1 activity levels (e.g., deacetylase activity). SIRT1 agonists useful for the methods described herein are also, ideally, biologically inactive or clinically inactive or tolerated with regard to other unwanted biological effects. The SIRT1 agonists described herein can be administered, for example, in prophylactically effective amounts in compositions designed to deliver the agonist in a prophylactically effective manner. The agonist, for example, can be delivered in a tissue-specific manner, in a dose-dependent manner, or as part of an extended release formulation.

The treatment(s) described herein are understood to utilize formulations including compounds identified herein or identified through methods described herein and, for example, salts, solvates and co-crystals of the compound(s). The compounds of the present disclosure can exist in unsolvated as well as solvated forms with pharmaceutically acceptable solvents such as, for example, water, ethanol, and the like. In general, the solvated forms are considered equivalent to the unsolvated forms for the purposes of the present disclosure.

The term “pharmaceutically acceptable salts, esters, amides and prodrugs” as used herein refers to those carboxylate salts, amino acid addition salts, esters, amides, prodrugs and inclusion complexes of the compounds of the present disclosure that are, within the scope of medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “solvate” refers to a compound in the solid state, wherein molecules of a suitable solvent are incorporated. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Co-crystals are combinations of two or more distinct molecules arranged to create a unique crystal form whose physical properties are different from those of its pure constituents (Remenar, J. et al., 2003. J. Am. Chem. Soc., 125:8456-7). Inclusion complexes are described in Remington: The Science and Practice of Pharmacy 19.sup.th Ed. (1995) volume 1, page 176-177. Examples of inclusion complexes are those with cyclodextrins, and all cyclodextrin complexes, natural and synthetic, with or without added additives and polymer(s), as described in U.S. Pat. Nos. 5,324,718 and 5,472,954. The disclosures of Remenar, Remington and the '718 and '954 patents are incorporated herein by reference in their entireties.

The compounds can be presented as salts. The term “pharmaceutically acceptable salt” refers to salts whose counter ion derives from pharmaceutically acceptable non-toxic acids and bases. Suitable pharmaceutically acceptable base addition salts for the compounds of the present disclosure include, but are not limited to, metallic salts made from aluminum, calcium, lithium, magnesium, potassium, sodium and zinc or organic salts made from lysine, N,N-dialkyl amino acid derivatives (e.g., N,N-dimethylglycine, piperidine-1-acetic acid and morpholine-4-acetic acid), N,N′-dibenzylethylenediamine, chloroprocaine, choline, diethanolamine, ethylenediamine, meglumine (N-methylglucamine) and procaine. Where the compounds contain a basic residue, suitable pharmaceutically acceptable base addition salts for the compounds include, for example, inorganic acids and organic acids. Examples include acetate, benzenesulfonate (besylate), benzoate, bicarbonate, bisulfate, carbonate, camphorsulfonate, citrate, ethanesulfonate, fumarate, gluconate, glutamate, bromide, chloride, isethionate, lactate, maleate, malate, mandelate, methanesulfonate, mucate, nitrate, pamoate, pantothenate, phosphate, succinate, sulfate, tartrate, p-toluenesulfonate, and the like (Barge, S. et al., J. Pharm. Sci., 66:1-19, 1977; the entire contents of which are incorporated herein by reference).

Diluents that are suitable for use herein include, for example, pharmaceutically acceptable inert fillers such as microcrystalline cellulose, lactose, sucrose, fructose, glucose dextrose, or other sugars, dibasic calcium phosphate, calcium sulfate, cellulose, ethylcellulose, cellulose derivatives, kaolin, mannitol, lactitol, maltitol, xylitol, sorbitol, or other sugar alcohols, dry starch, saccharides, dextrin, maltodextrin or other polysaccharides, inositol or mixtures thereof. The diluent can be, for example, a water-soluble diluent. Examples of diluents include, for example: microcrystalline cellulose such as Avicel PH112, Avicel PH101 and Avicel PH102 available from FMC Corporation; lactose such as lactose monohydrate, lactose anhydrous, and Pharmatose DCL 21; dibasic calcium phosphate such as Emcompress; mannitol; starch; sorbitol; sucrose; and glucose. Diluents are carefully selected to match the specific composition with attention paid to the compression properties. The diluent can be used in an amount of about 2% to about 80% by weight, about 20% to about 50% by weight, or about 25% by weight of the treatment formulation.

Other agents that can be used in the treatment formulation include, for example, a surfactant, dissolution agent and/or other solubilizing material. Surfactants that are suitable for use herein include, for example, sodium lauryl sulphate, polyethylene stearates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxamers, polyvinyl alcohol and sorbitan fatty acid esters.

Dissolution agents increase the dissolution rate of the active agent and function by increasing the solubility of the active agent. Suitable dissolution agents include, for example, organic acids such as citric acid, fumaric acid, tartaric acid, succinic acid, ascorbic acid, acetic acid, malic acid, glutaric acid and adipic acid, which may be used alone or in combination. These agents can also be combined with salts of the acids, e.g., sodium citrate with citric acid, to produce a buffer system. Other agents that can be used to alter the pH of the microenvironment on dissolution include salts of inorganic acids and magnesium hydroxide.

Disintegrants that are suitable for use in the pharmaceutical composition of the present disclosure include, for example, starches, sodium starch glycolate, crospovidone, croscarmellose, microcrystalline cellulose, low substituted hydroxypropyl cellulose, pectins, potassium methacrylate-divinylbenzene copolymer, poly(vinyl alcohol), thylamide, sodium bicarbonate, sodium carbonate, starch derivatives, dextrin, beta cyclodextrin, dextrin derivatives, magnesium oxide, clays, bentonite and mixtures thereof.

A SIRT1 agonist used in a formulation as described herein is the “active agent” or “active ingredient” of the formulation. The active ingredient(s) described herein can be mixed with excipients, which are pharmaceutically acceptable and compatible with the active ingredient, in amounts suitable for use in the treatment methods described herein. Various excipients can be homogeneously mixed with the active agent as would be known to those skilled in the art. The active agent, for example, can be mixed or combined with excipients such as but not limited to microcrystalline cellulose, colloidal silicon dioxide, lactose, starch, sorbitol, cyclodextrin and combinations of these.

Compositions of the present disclosure can also optionally include other therapeutic ingredients, anti-caking agents, preservatives, sweetening agents, colorants, flavors, desiccants, plasticizers, dyes, and the like.

The following examples provide illustrative descriptions of the mechanism(s) involved in induction of premature senescence in BRCA1^{mut/+} HMECs, as well as, the cell-type specificity of this phenotype by studying the growth and senescence of keratinocytes and skin fibroblasts from BRCA1-mutation carriers. The identification of SIRT1 as a mediator of HIS and genomic instability is shown. These examples underscore the importance of the materials and methods described herein for BRCA1 in breast biology and tissue-specific tumorigenesis.

### EXEMPLIFICATION

### Example 1

To determine the cell type-specificity of BRCA1 haploinsufficiency and whether this relates to the rapid onset of breast tumors, various cell types isolated from disease-free tissues of women harboring deleterious mutations in BRCA1 (BRCA1^{mut/+}) were studied. This approach has provided novel insights into a breast epithelial-specific role of BRCA1 in regulating cellular senescence and immortalization.

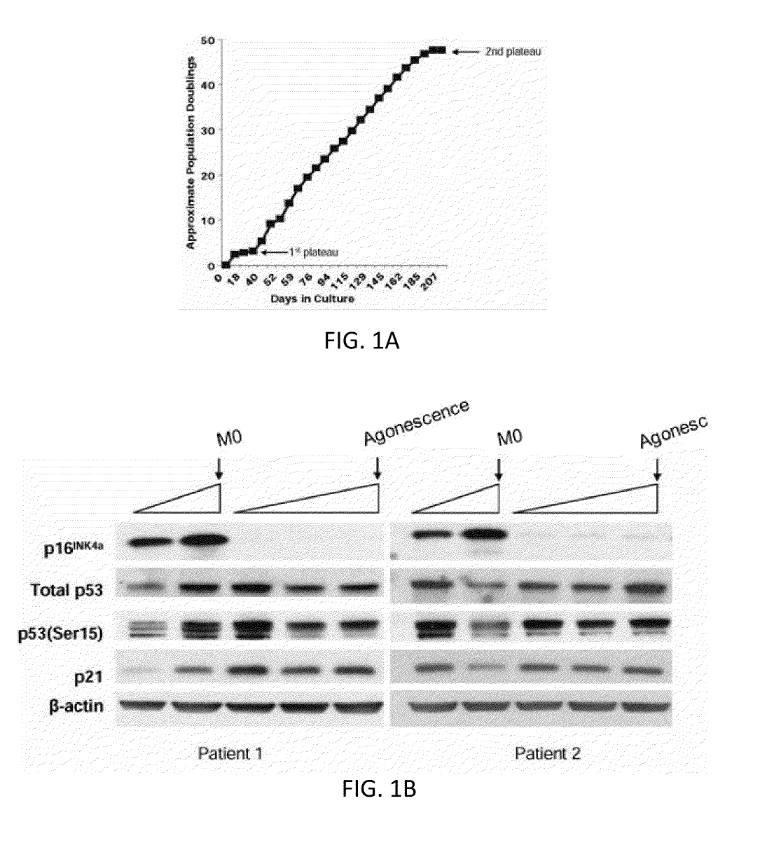

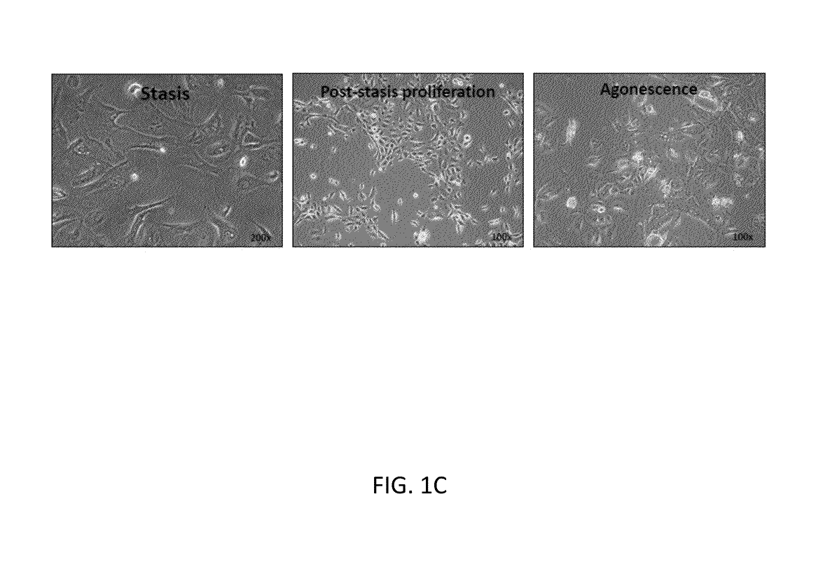

Context-specificity of BRCA1 haploinsufficiency in promoting premature senescence. BRCA1^{mut/+} breast epithelial cells but not fibroblasts undergo premature senescence (months 1-24). To further evaluate how BRCA1 haploinsufficiency is associated with increased predisposition to cellular transformation, normal human mammary epithelial cell (HMEC) behavior was investigated ex vivo. The HMEC culture model (Garbe, J. et al., Cancer Res., 69:7557-68, 2009; Stampfer, M. & Bartley, J., Proc. Natl. Acad. Sci. USA, 82:2394-8, 1985; Huschtscha, L. et al., Cancer Res., 58:3508-12, 1998; Romanov, S. et al., Nature, 409:633-7, 2001) was used to study the properties of primary HMECs from WT and BRCA1-mutation carriers. Notably, WT HMECs encounter two mechanistically distinct senescent-like barriers when cultured ex vivo with this model (FIG. 1A; Hammond, S. et al., Proc. Natl. Acad. Sci. USA, 81:5435-9, 1984; Brenner, A. et al., Oncogene, 17:199-205, 1998; Novak, P. et al., Cancer Res., 69:5251-8, 2009; Holst, C. et al., Cancer Res., 63:1596-601, 2003; Dumont, N. et al., Breast Cancer Res., 11:R87, 2009).

The first proliferative barrier, referred to as stasis or M0, is associated with the classical stress-induced senescence morphology and senescence-associated β-galactosidase (β-gal) activity (Garbe, J. et al., Cell Cycle, 6:1927-36, 2007). The molecular characteristics of cells approaching and in M0/stasis are consistent with an Rb-mediated growth arrest mediated by increased expression of p16/INK4a, but also consistent with the activation of the p53 pathway (FIG. 1B). Although p53 expression is induced during this growth arrest period, bypass of this proliferative barrier only occurs upon suppression of the Rb pathway, indicating that Rb activity is the main mediator of regulating this stress-induced proliferative barrier. Indeed, HMECs that escape this proliferative barrier invariably demonstrate inactivation of p16/INK4A through DNA promoter methylation and resume a period of active proliferation for an additional 30-50 population doublings (FIG. 1).

Cells that emerge from this Rb-dependent M0 barrier rapidly proliferate until they reach a second senescence barrier referred to as agonescence (FIG. 1). The molecular characteristics of cells in agonescence are consistent with a p53-mediated growth arrest, rather than an Rb-mediated arrest, due to activation of DNA damage responses (DDR) and genomic instability as a consequence of telomere attrition and dysfunction. This second barrier of cell growth is also associated with senescence morphology and senescence-associated β-gal activity. Unlike the first proliferative barrier, however, cells that are forced to continue to proliferate as a result of p53 loss eventually undergo apoptosis or become non-proliferative as a consequence of crisis due to the genomic instability and mitotic catastrophes from critically short telomeres. Notably, it has never been reported that HMECs that have entered into this second barrier of cell growth can spontaneously immortalize. Thus, cellular immortalization has been proposed to be the rate limiting step during neoplastic transformation of HMECs.

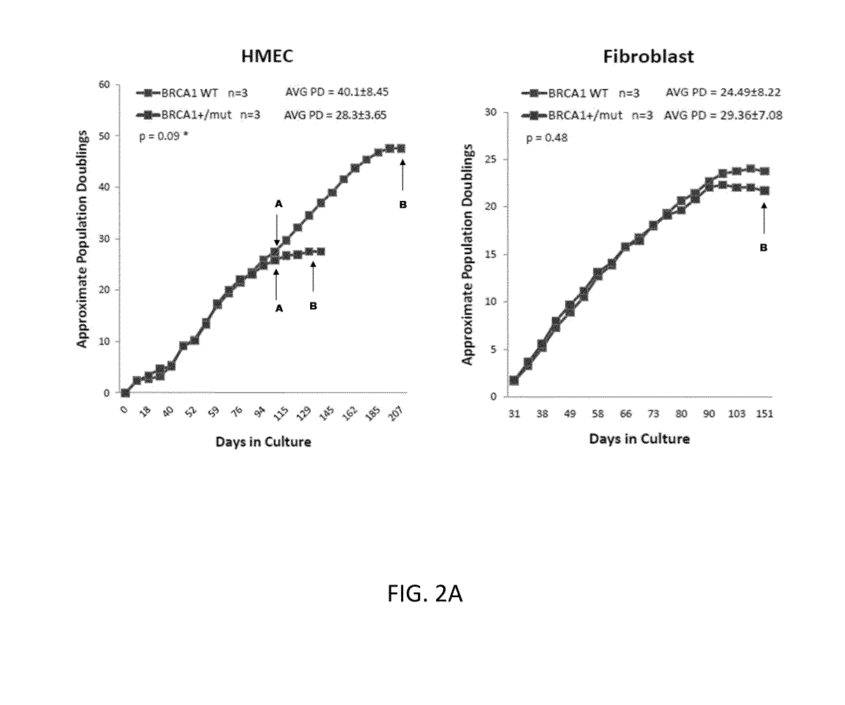

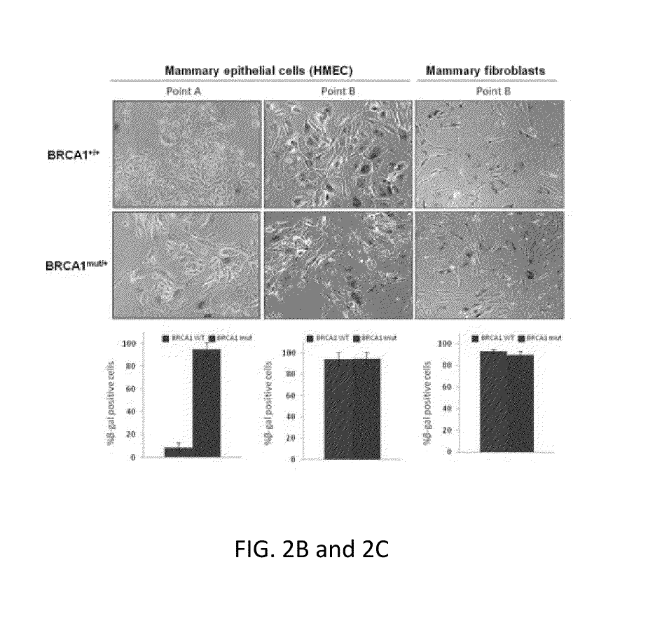

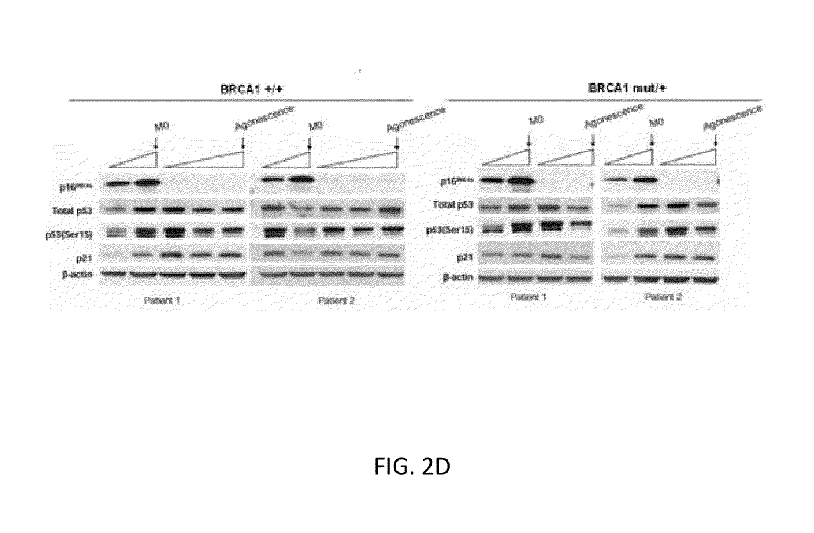

Examination of age-matched HMECs from disease-free BRCA1 prophylactic mastectomy tissues compared to HMECs from disease-free reduction mammoplasty tissues from non-BRCA1 carriers revealed similar growth kinetics in early cultures (FIG. 2A). Both WT and BRCA1^{mut/+} HMECs rapidly entered into M0 and induced p16/IKN4a and p53 protein expression in a similar fashion (FIG. 2D). In addition, WT and BRCA1^{mut/+} HMECs overcame M0 with similar frequencies and efficiencies, and both exhibited loss of p16/INK4a expression (FIG. 2D). In contrast to WT HMECs, however, BRCA1^{mut/+} HMECs stopped proliferating after −28 population doublings (FIG. 2A). This premature growth arrest was observed across different patient-derived BRCA1^{mut/+} HMECs (N=3, p=0.003) and was associated with a senescent phenotype characterized by large, flat morphology and positive staining for SA-β-gal (FIGS. 2B and 2C).

To determine whether premature senescence was a general consequence of BRCA1 haploinsufficiency, the growth kinetics of normal breast fibroblasts isolated from age-matched disease-free BRCA1 prophylactic mastectomy tissues and reduction mammoplasty tissues were also analyzed. WT (N=3) and BRCA1^{mut/+} (N=3) human breast fibroblasts underwent-25-30 population doublings in culture, after which most of the cells became β-gal positive and stopped dividing (p=0.48) (FIG. 2). These findings demonstrate that haploinsufficiency for BRCA1 in HMECs but not mammary fibroblasts results in the engagement of a premature senescence-like barrier.

Senescence in BRCA1^{Mut/+} HMECs is Molecularly Different from Agonescence in WT Cells.

Although there are no reports of premature senescence-associated phenotypes due to BRCA1 haploinsufficiency, MEFs deficient in BRCA1 have been reported to undergo premature senescence in culture (Cao, L. et al., Genes Dev., 17:201-213, 2003). Senescence in BRCA1-null cells is triggered in response to excessive DDR through a p53-dependant response.

To determine whether premature senescence in BRCA1^{mut/+} HMECs is due to increased telomere attrition or elevated DDR compared to WT cells, chromosomal rearrangements using cytogenetic analysis, DNA-damage foci formation, phosphorylation of H2AX, as well as phosphor-53 (Ser15) levels were assessed in proliferating (pre-senescence) and senescent/agonescent HMECs.

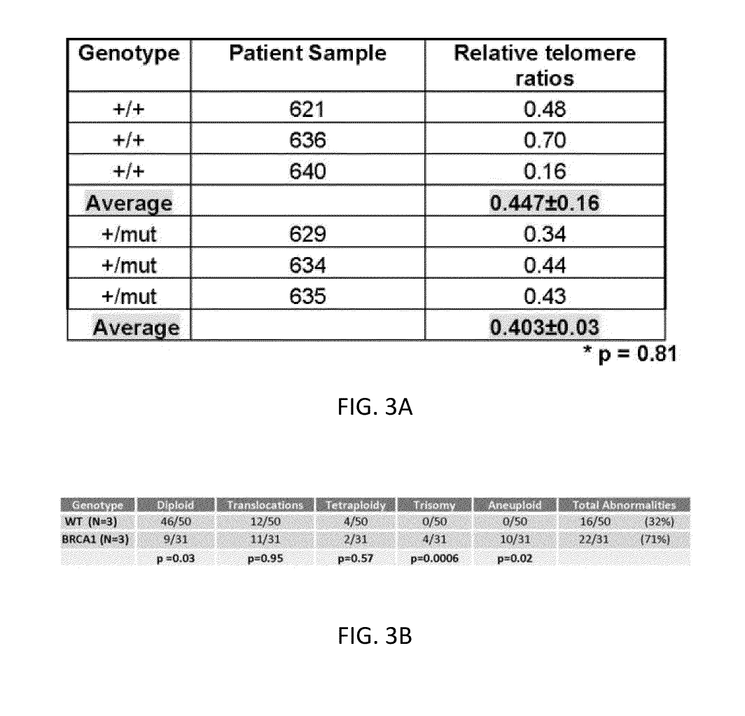

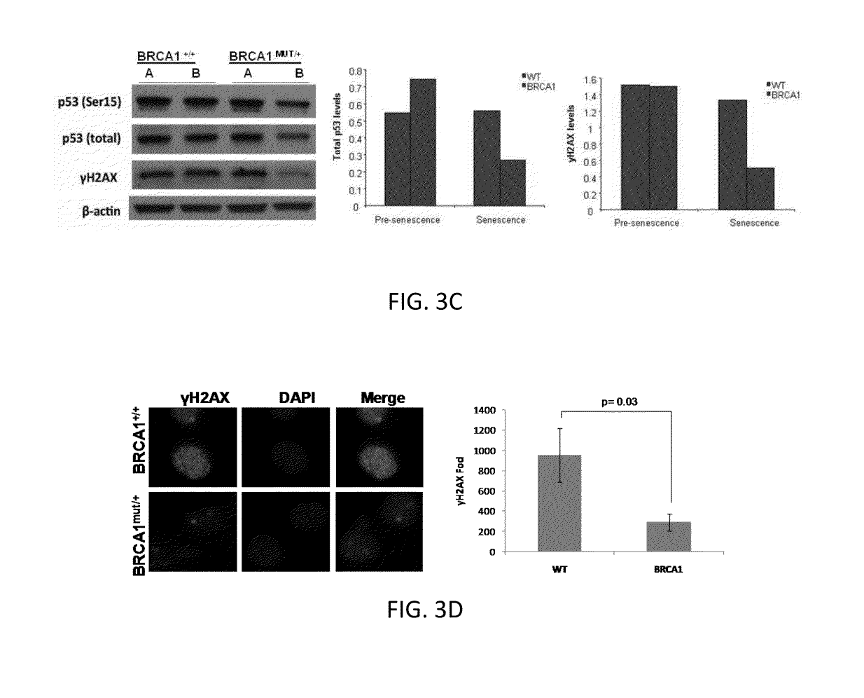

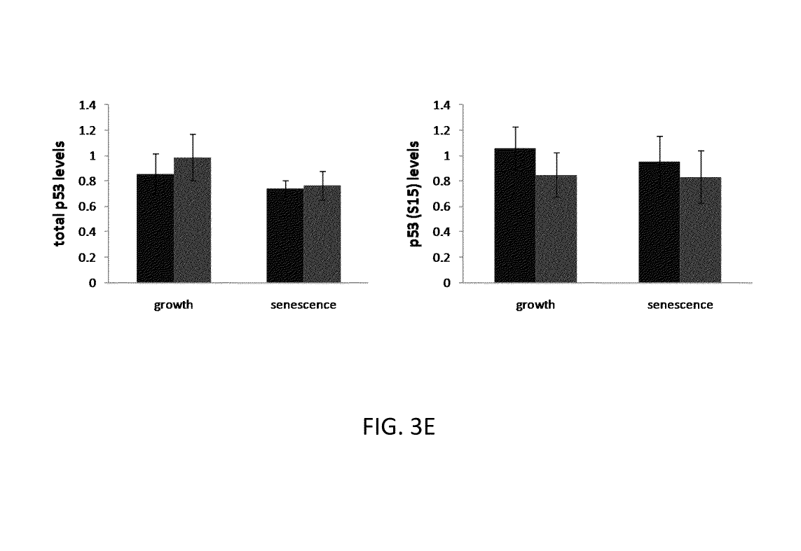

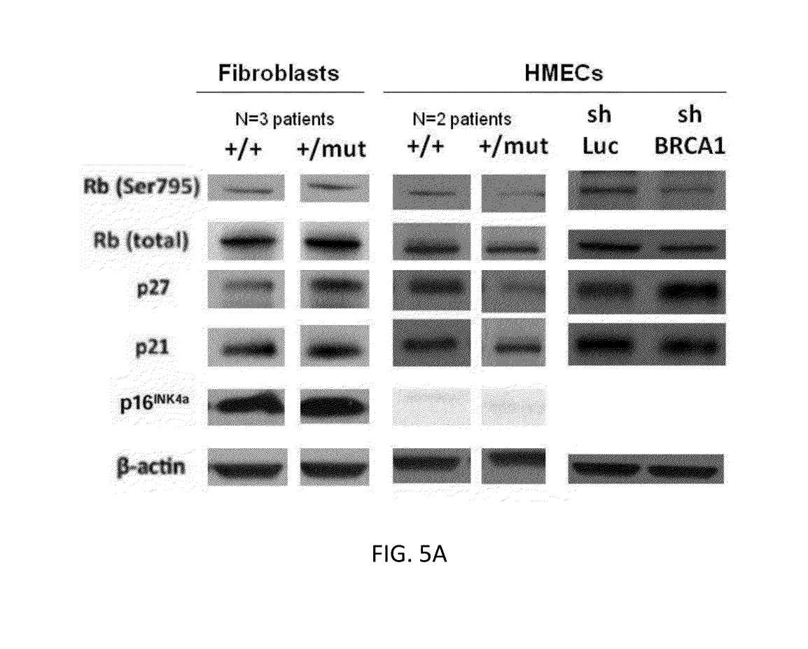

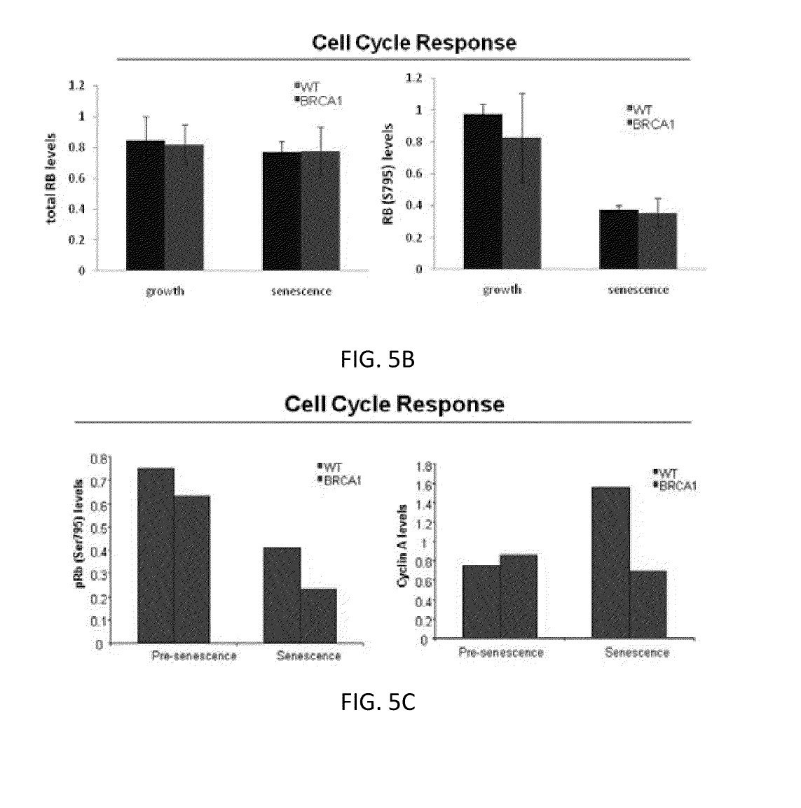

Proliferating cultures of BRCA1^{mut/+} HMECs exhibited increased genomic instability and chromosomal abnormalities (FIG. 3B) compared to WT HMECs, but did not differ in their average telomere lengths or expression levels of DDR mediators such as γH2AX and phospho-p53 (Ser15) (FIGS. 3A, 3B and 3D). At senescence BRCA1^{mut/+} HMECs surprisingly exhibited reduced total levels of p53 and γH2AX compared to either agonescent WT HMECs or senescent fibroblasts (FIGS. 3C and 3E). This is consistent with findings in which BRCA1 complexes are required for p53 phosphorylation at Ser15 upon ionizing radiation-induced DNA damage (Fabbro, M. et al., J. Biol. Chem., 279:31251-8, 2004). Furthermore, at senescence BRCA1^{mut/+} HMECs showed significantly reduced numbers of cells with large γH2AX foci as compared to WT HMECs (FIG. 3). Similar findings were observed following knockdown of BRCA1 in WT HMECs by shRNA (FIG. 5).

Premature Senescence in BRCA1^{Mut/+} HMECs is not Associated with BRCA1 LOH.

Loss of tumor suppressor genes (e.g., VHL, PTEN or NF1) can lead to the induction of premature senescence programs in part through inactivation of cyclin-CDK complexes, thereby leading to reduced phosphorylation of Rb (Berger, A. et al., Nature, 476:163-9, 2011; Kuilman, T. et al., Genes Dev., 24:2463-79, 2010; Young, A. et al., Nat. Cell Biol., 10:361-9, 2008).

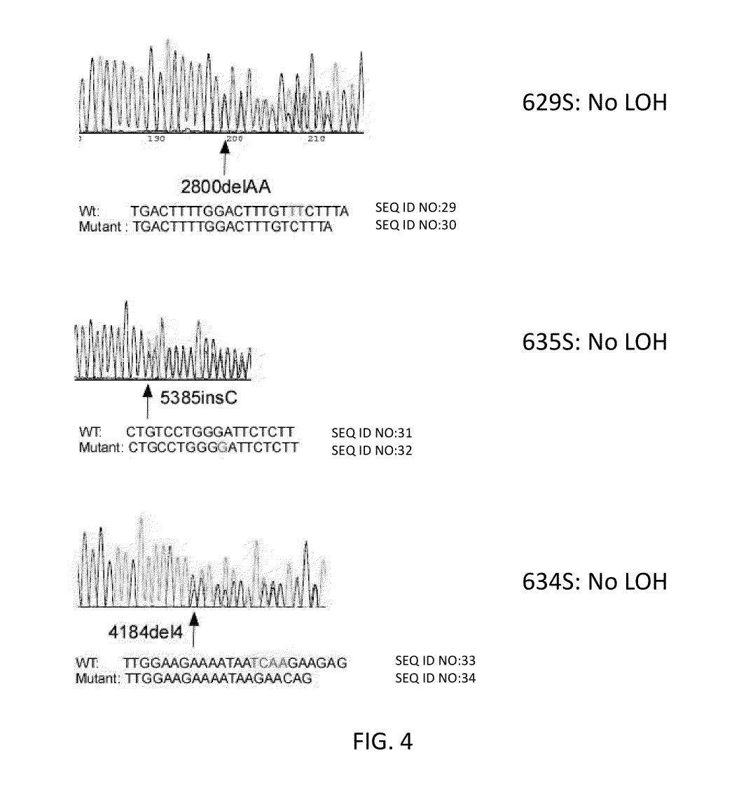

Multiplex ligation-dependent probe amplification and sequencing was used to determine if loss of heterozygosity (LOH) leads to premature senescence in BRCA1^{mut/+} HMECs. Both the WT and mutant alleles for BRCA1 were present in all proliferative and in all senescent BRCA1^{mut/+} HMECs (FIG. 4).

These findings indicate that BRCA1-induced senescence in HMECs is not due to increased DDR or LOH of BRCA1, supporting a novel context-specific mechanism.

### Haploinsufficiency-Induced Senescence (HIS).

Findings described herein have revealed that breast epithelial cells haploinsufficient for BRCA1 undergo premature senescence rather than a p53-mediated DNA damage-induced apoptosis. This premature growth arrest is unique to epithelial cells as breast fibroblasts harboring mutations in BRCA1 do not undergo premature senescence.

Shown herein are results addressing whether inherited mutations in BRCA1 in other cell types lead to premature senescence, and whether senescence occurs in vivo in BRCA1-mutation carriers. In addition, DDR and p53 pathways in BRCA1^{mut/+} cells from other tissues are examined as well as genomic instability in BRCA1^{mut/+} HMECs.

To determine whether epithelial cells or fibroblasts from BRCA1-mutation carriers undergo premature senescence, proliferation and senescence of keratinocytes, additional HMECs, skin and mammary fibroblasts are analyzed. Primary keratinocytes and corresponding isogenic fibroblasts from skin tissues derived from five BRCA1^{+/+} and BRCA1^{mut/+} patient samples are used. Two additional primary HMECs and corresponding isogenic fibroblasts from BRCA1^{+/+} and BRCA1^{mut/+} patient samples also used. BRCA1 mutation status in all patient samples is confirmed prior to analysis.

Skin tissues are digested in dispase overnight and the epidermis is separated from the dermis. Epidermal tissue is enzymatically digested; aggregates are filtered through a 40 micron mesh to generate a single cell suspension. 2×10⁵ cells are seeded in serum-free keratinocyte culture medium in 10 cm plates. Dermal tissues are also enzymatically digested, filtered and 2×10⁵ fibroblasts are seeded in serum containing medium. Population doublings (PD) are calculated for both keratinocytes and fibroblasts until PD=0 for 3 sequential passages. To calculate PD, attachment efficiency is determined by counting attached cells 12 h after plating. The number of accumulated PD per passage is determined using the equation, PD=log [A/(BC)]/log 2, where A is the number of collected cells, B is the number of plated cells, and C is the attachment efficiency. The percent cells at senescence is quantified when PD<2 and PD=0 by SA-β-gal activity at pH 6.0. Growth curves and SA-β-gal positivity is compared between WT control cells and BRCA1^{mut/+} derived cells.

### DDR, Senescence-Associated DNA-Damage Foci, and p53 Pathway Activation.

Proliferating and senescent BRCA1 WT and BRCA1^{mut/+} keratinocytes, additional HMECs, and fibroblasts were examined for phosphorylation of H2AX, phospho-53 (Ser15), total levels of p53, CHK1, phospho-CHK1 (Ser345), phospho-CHK2 (Thr68) as well as ATM/ATR substrates using anti-phospho-ST/Q. In addition, TUNEL analysis was performed in cells undergoing senescence for the presence of DNA breaks while BP53 staining in proliferating cells is used to assess DNA damage associated with stalled replication forks. The specificity of these pathway components was confirmed using WT fibroblasts treated with UV or γIR, respectively. To confirm genetic instability at a more global level, SNP array profiles of proliferating and senescent BRCA1^{mut/+} and BRCA1^{+/+} cells ire analyzed.

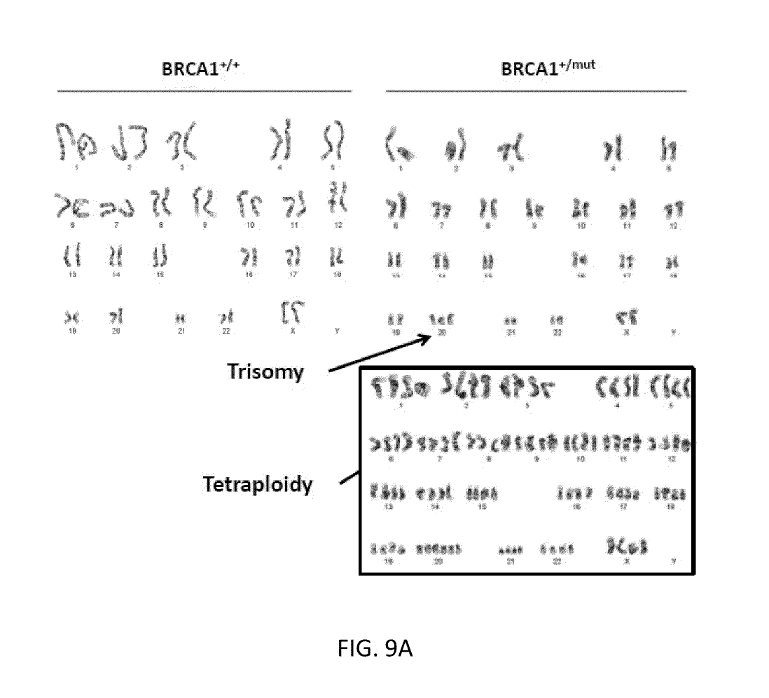

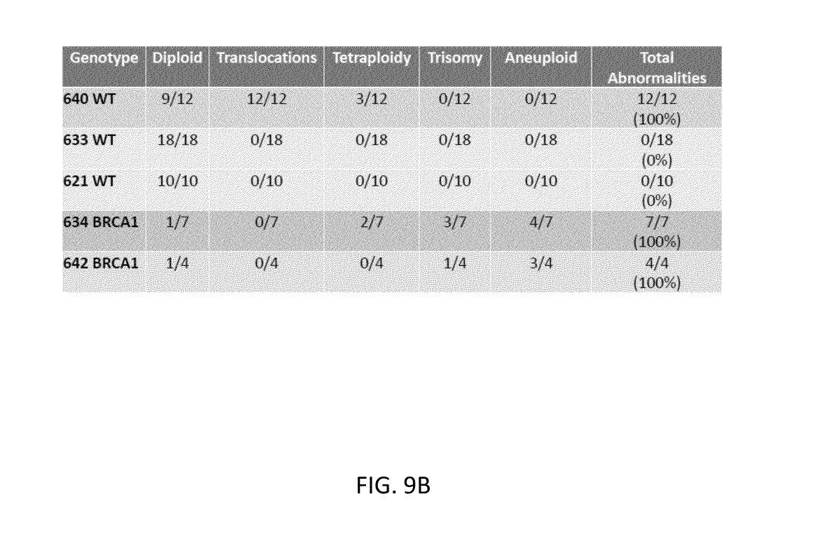

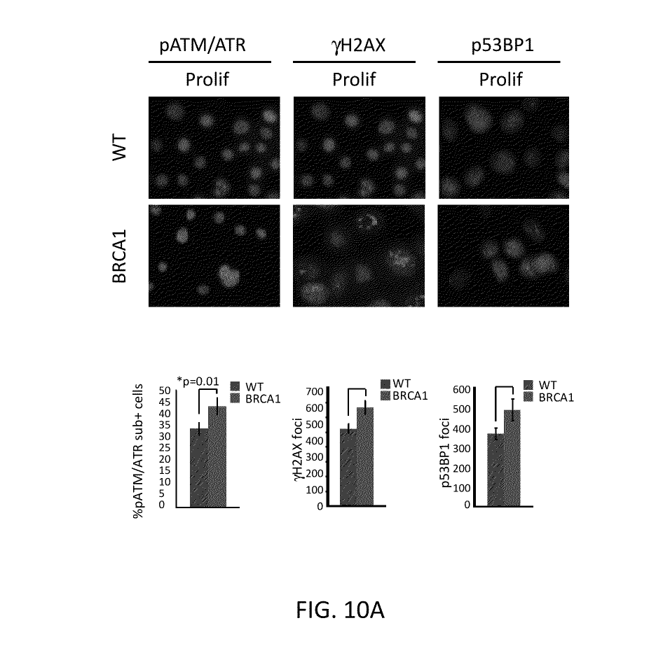

Throughout the culture of post-stasis BRCA1^{mut/+} HMECs, there is an increase in the frequency of metaphase cells with aberrations compared to WT HMECs. The most numerous chromosomal abnormalities in BRCA1^{mut/+} HMECs are tetraploidy, trisomy, aneuploidy as well as translocations at particular chromosomes arms (FIG. 9). These results indicate defects in centrosomes and telomeric DNA are responsible for increased genomic instability. Therefore, centrosomes of BRCA1^{mut/+} HMECs are examined to determine whether supernumerary centrosomes drive genomic instability in BRCA1^{mut/+} HMECs. Using an antibody that recognizes γ-tubulin, a component of pericentriolar material, and an antibody that recognizes centrin, a component of centrioles, proliferating and senescent BRCA1^{mut/+} HMECs are compared to WT HMECs.

One mechanism by which excessive duplication of centrosomes can arise is by uncoupling of the centrosome duplication and DNA replication cycles. To investigate this mechanism, BRCA1^{mut/+} HMECs are treated to achieve a transient inhibition of DNA synthesis with hydroxyurea (HU), a reversible inhibitor of DNA synthesis, that allows the cells to transition into S-phase of the cell cycle but prevents them from progressing through S-phase. BRCA1-associated senescence-like growth arrest in vivo during tumor progression in BRCA1^{mut/+} tissue recombinants.

An approach to create orthotopic humanized tissue-transgenic breast cancers using mammary epithelial cells obtained from prophylactic mastectomy tissues from patients harboring deleterious mutations in BRCA1 was developed. This method involves three distinct temporal steps: (1) clearing of the murine mammary fat pad, (2) reconstitution of the mammary fat pad with human stromal cells and (3) introduction of lentiviral-infected mammary epithelial cells co-mixed with activated fibroblasts into the humanized fat pad. Because this system does not require any intervening cell culture, possible in vitro imposed alterations are effectively minimized. This model system also captures the natural developmental history of breast cancer and can be used to facilitate the study of breast cancer progression (Wu, M. et al., Proc. Natl. Acad. Sci. USA, 106:7022-7, 2009). Tumors that arise from this model exhibit robust activation of hTERT gene expression, suggesting that bypass of possible senescent barriers and acquisition of cellular immortalization may also be necessary events that can be captured in vivo.

This model is utilized to create tumors from histologically normal human breast tissues obtained from BRCA1 mutation carriers and to assess both SA-β-gal staining during tumor progression as well as BRCA1 LOH.

BRCA1-associated and non-BRCA1-associated breast cancers were created using various combinations of transforming oncogenes that disrupt the major pathways necessary for neoplastic transformation. By enumerating the temporal progression of cancer development in vivo, it was found that on day 7 post-implantation, mammary epithelial cells are undergoing morphogenesis to form human breast acini, which by day 10 have matured to the stage of lumen formation and primitive ductal outgrowth. By day 17 after infection/implantation, DCIS-like outgrowths from breast tissue recombinants become readily detectable. By day 35 after infection, invasive carcinomas become visible. Transforming oncogenes (e.g., mutant p53, (p53R175H), overexpression of cyclin E, overexpression of EGFR, and an oncogenic form of K-ras (RasG12V) (Foulkes, W., J. Med. Genet., 41:1-5, 2003)) commonly found in BRCA1-associated breast cancers are utilized for this study. Reconstituted BRCA1^{mut/+} human breast tissue from 10 tissue recombinants are assessed at each time point on days 7, 10, 17, 25, 35 and 45 after implantation, and tissues are assessed for SA-β-gal staining, p16 expression and BRCA LOH.

### BRCA1-Associated Senescence-Like Growth Arrest In Vivo in Patient-Derived BRCA1^{Mut/+} Tissues.

The in vivo relevance and specificity of senescence in BRCA1-mutation carriers is assessed in normal skin, normal breast tissue and breast cancer tissues from BRCA1-mutation carriers. Cancer-associated and non-cancer-associated regions of whole breast tissues are collected and dissected immediately following mastectomy surgery. Cancer-associated regions are selected to be minimally necrotic regions of the tumor mass, while histologically normal tissue is examined that is physically adjacent to the tumor mass, as well as normal tissue at least 2 cm away from the tumor tissue but in the affected breast. Tissues are embedded in OCT and sectioned. Normal, uninvolved breast skin is collected from the mastectomy surgery. SA-β-gal staining is performed on frozen tissue sections (Dimri, G. et al., Proc. Natl. Acad. Sci. USA, 92:9363-7, 1995; Courtois-Cox, S. et al., Cancer Cell, 10:459-72, 2006).

Tissues with other markers of senescence are co-stained with IHC, or, when necessary, in serial sections such as p16 and γH2AX foci. To determine whether SA-β-gal-expressing cells have retained the WT BRCA1 allele, laser capture microdissection (LCM) of SA-β-gal positive cells as well as SA-β-gal positive negative cells adjacent to the tumor, and tumor cells in which LOH of BRCA1 has likely occurred are extracted and analyzed for LOH using multiplex ligation-dependent probe amplification and sequencing. Known BRCA1 tumor tissues in which LOH has already been evaluated is used as a positive control and normal reduction mammoplasty tissues from individuals with WT BRCA1 serves as a negative control for both SA-β-gal staining and BRCA1 LOH.

### Haploinsufficiency and Genomic Instability.

Increased DDR and p53 pathway activation has traditionally been associated with BRCA1 loss in a cell type independent manner. Mutation of a single allele of BRCA1 in HMECs, but not other cell types, is shown herein to be associated with significant chromosomal rearrangements and aneuploidy (FIG. 3), but not increased p53 pathway activation. This difference in DDR and a dampened p53 pathway marks cell type specific responses due to BRCA1 haploinsufficiency.

Using a gene-targeting approach in immortalized HMECs, mutation of a single allele of BRCA1 was reported to lead to genomic instability (Konishi, H. et al., Proc. Natl. Acad. Sci. USA, 108:17773-8, 2011). Consistent with this finding, described herein are data showing BRCA1^{mut/+} HMECs exhibit significant chromosomal rearrangements and aneuploidy (FIG. 3). Increased genomic instability associated with BRCA1 haploinsufficiency is a unique and defining feature of HMECs compared to other cell types. These results demonstrate for the first time a tissue-specific difference either at the level of genomic instability or premature senescence associated with BRCA1 haploinsufficiency.

### Haploinsufficient BRCA1 Affects SIRT1 and Rb.

Although cellular senescence (either oncogene-induced senescence, replicative senescence, or even some forms of tumor suppressor loss-induced senescence) can be regulated by p53 activation, it is also regulated by the inactivation of Rb. Data presented herein indicate that premature senescence in BRCA1^{mut/+} HMECs is associated with reduced p53 activation, showing that cell cycle inhibition in these cells occurs through a mechanism that does not involve p53 (FIGS. 1D and 3C). Total levels of Rb were similar in senescent BRCA1 mut/+HMECs and fibroblasts compared to WT HMECs and fibroblasts, but levels of phosphorylated Rb at Ser795 were significantly reduced only in senescent BRCA1^{mut/+} HMECs (FIGS. 5A and 5C). In addition, E2F expression and the E2F target gene, cyclin A (mRNA and protein) were both decreased in senescent BRCA1^{mut/+} HMECs compared to either WT HMECs or fibroblasts (FIG. 5C). The levels of other cell cycle inhibitors including p16INK4a, p21Cip1A/Waf1, p27 and cyclin E, however, did not appear to differ in expression between WT, BRCA1^{mut/+} HMECs or fibroblasts (FIG. 5A). Similar findings were observed in senescent WT HMECs upon knockdown of BRCA1 shRNA (FIG. 5A). These are indicative of a mechanism in which senescence in BRCA1^{mut/+} HMECs is mediated through inactivation of Rb as a consequence of decreased cyclinA-CDK2 activity and reduction of E2F expression thereby halting cell cycle progression.

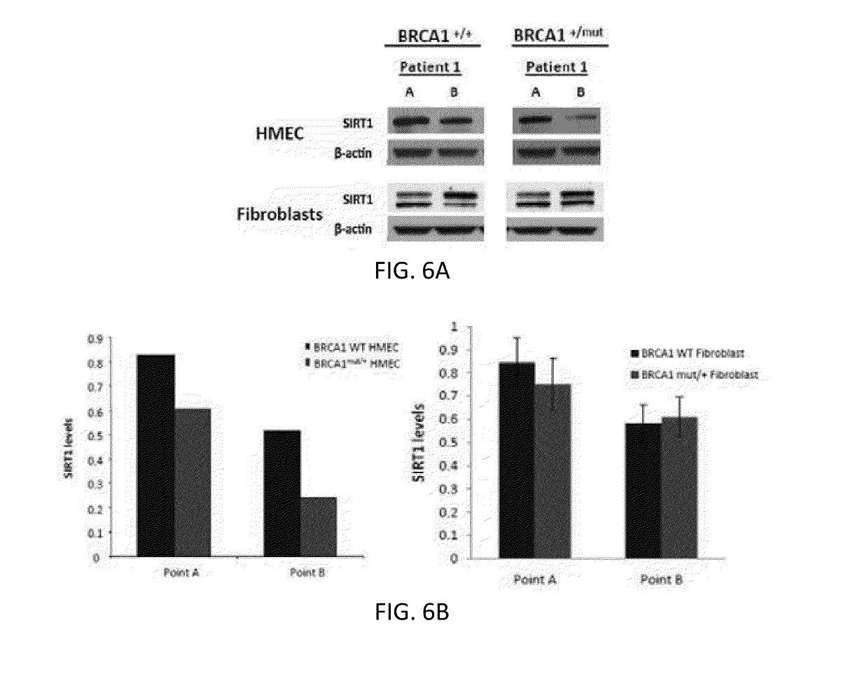

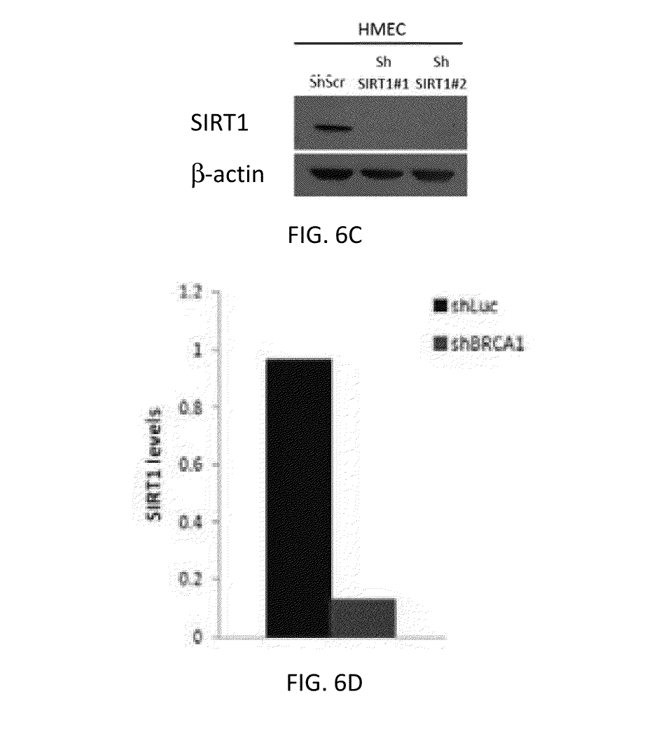

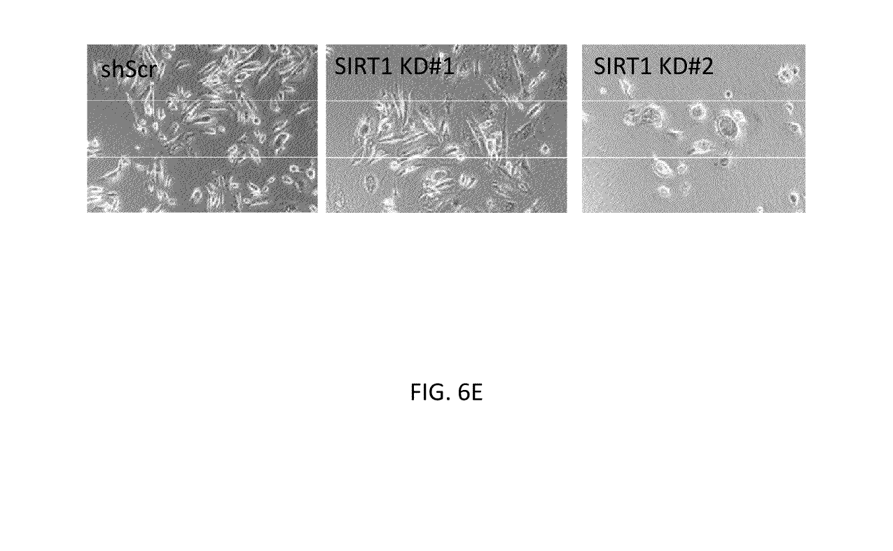

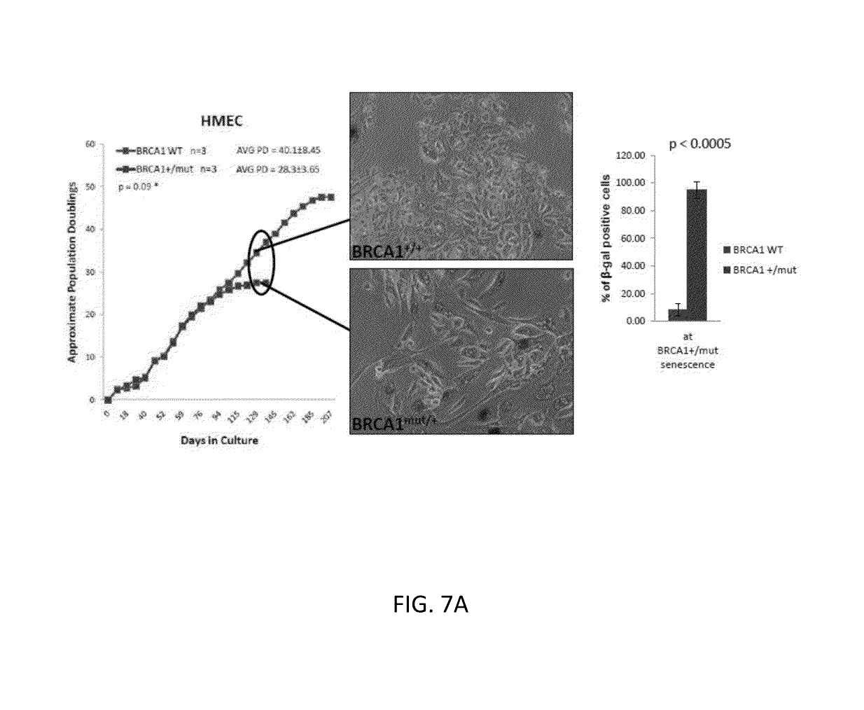

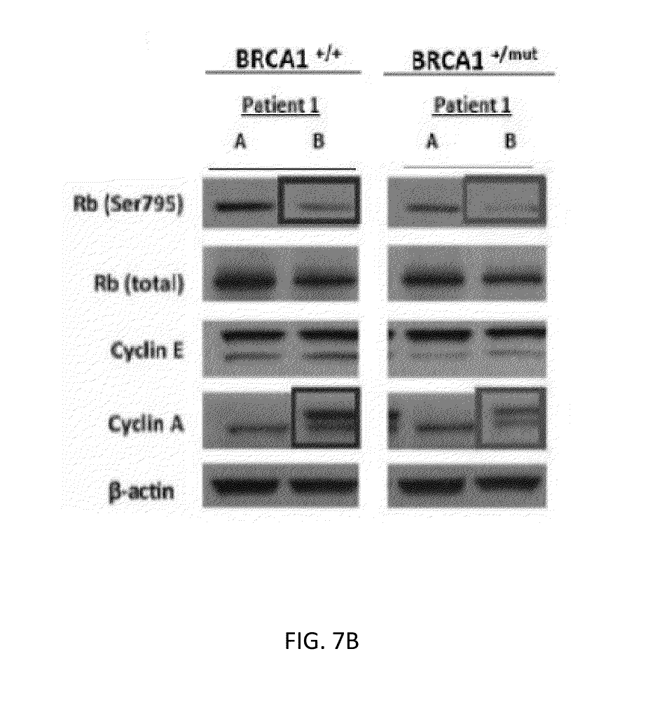

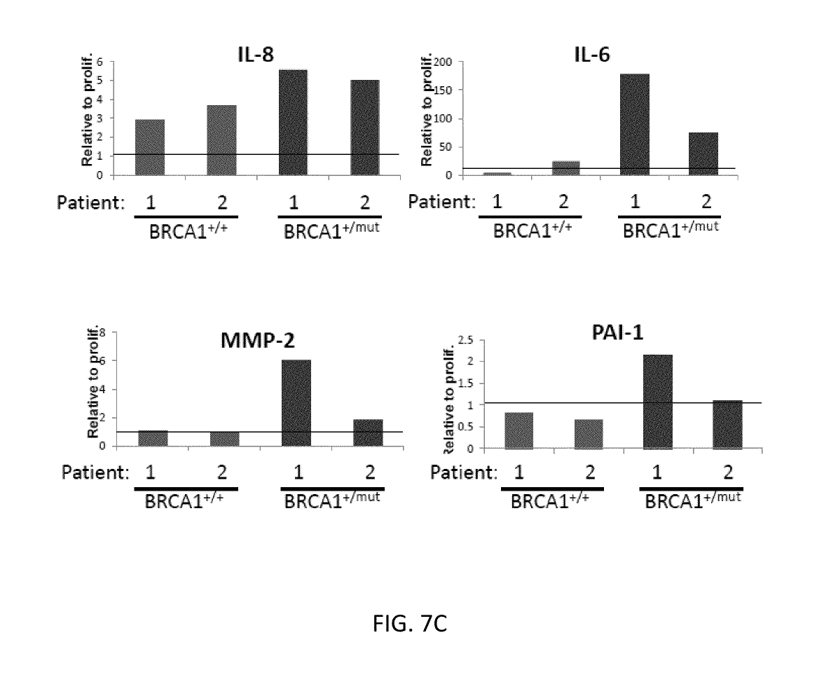

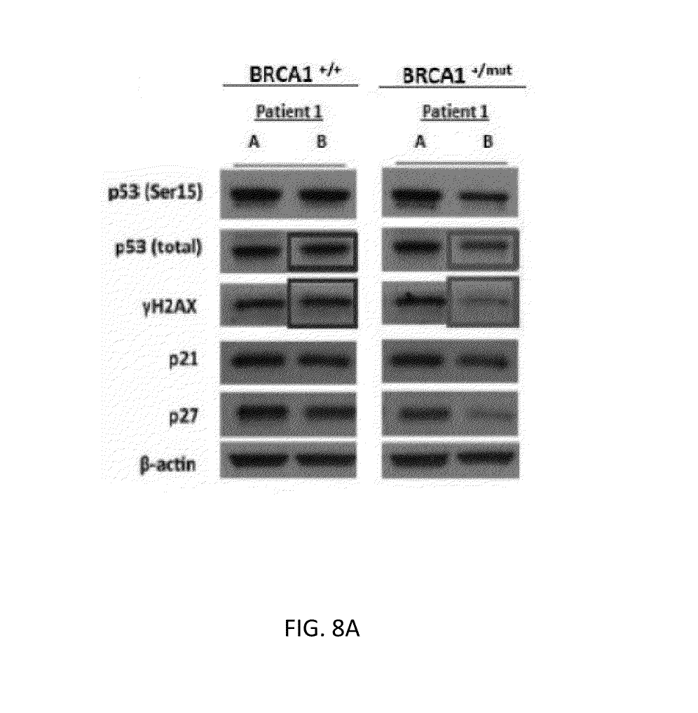

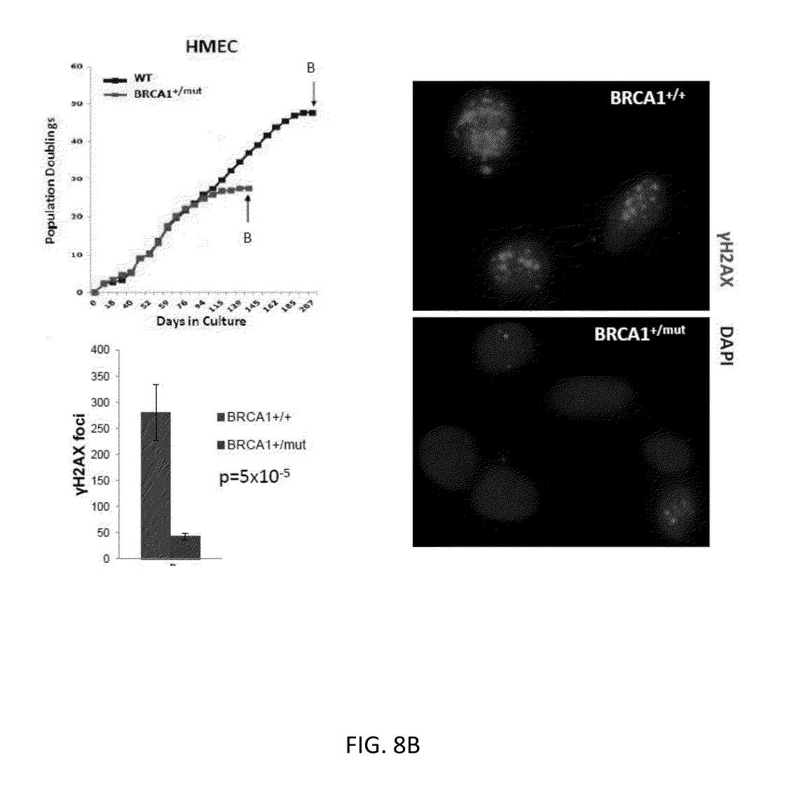

In addition to phosphorylation, the activity of Rb is also regulated by acetylation events on multiple residues that can be catalyzed by the deacetylase functions of the NAD-dependent deacetylase SIRT1 in Rb-SIRT1 complexes during S-phase (Wong, S. & Weber, J., Biochem. J., 407:451-60, 2007). SIRT1 protein expression decreases during replicative senescence and there is a negative correlation between levels of SIRT1 and SA-β-gal activity (Langley, E. et al., EMBO J., 21:2383-96, 2002; Huang, J. et al., PLoS One, 3:e1710, 2008). Cell cycle arrest in these settings is associated with both decreased Rb phosphorylation and increased Rb acetylation. Consistent with these findings, reduced levels of SIRT1 in HMECs from BRCA1-mutation carriers was observed prior to senescence (FIGS. 6A and 6B) as well as in HMECs in which BRCA1 expression had been inhibited through lentiviral-mediated short hairpin knockdown (FIG. 6B). In BRCA1^{mut/+} HMECs and shBRCA1 HMECs, decrease in SIRT1 expression is associated with decreased Rb phosphorylation and increased Rb acetylation. In addition, shRNA experiments inhibiting SIRT1 using lentiviral-mediated shRNA silencing in WT HMECs resulted in immediate cell cycle arrest and morphological changes associated with senescence (FIG. 6C-6E).

In addition to mediating deacetylation of Rb, SIRT1 regulates homologous recombination at telomeres, centromeres and chromosome arms, and specifically binds to telomeric repeats to influence telomere length. SIRT1 mediates deacetylation of telomeric and pericentromeric regions, thereby leading to shorter telomeres. Telomere erosion rates were therefore examined in WT and BRCA1^{mut/+} HMECs. Telomere length of BRCA1^{mut/+} and WT HMECs was assessed using a modified quantitative PCR method (Cawthon, R., Nucleic Acids Res., 30:e47, 2002; Getliffe, K. et al., Aliment. Pharmacol. Ther., 21:121-31, 2005; Gil, M. et al., Mol. Biotechnol., 27:169-72, 2004; Martin-Ruiz, C. et al., Brain Res. Mol. Brain. Res., 123:81-90, 2004). This technique measures the factor by which the ratio of telomere repeat copy number to single-gene copy number differs and generates a relative Telomere/Single Copy Gene (T/S) ratio that is proportional to average telomere length.

Data show BRCA1^{mut/+} HMECs undergo more rapid telomere erosion as they approach senescence compared to WT cells approaching agonescence. BRCA1^{mut/+} HMECs also exhibit significant chromosomal rearrangements and/or duplications of chromosomal arms including 13q34, 3p and the long arm of chromosome 11 (FIG. 3B). These findings show that a lack of SIRT1 or reduced SIRT1 activity in BRCA1^{mut/+} HMECs results in both modifications of Rb acetylation leading to induction of senescence, as well as changes in DNA acetylyation at telomeric regions resulting in telomere dysfunction and increased genomic instability.

Findings described herein show premature senescence in BRCA1^{mut/+} HMECs is not mediated by increased p53 signaling or DDR, but rather through modulation of Rb activity. It is herein demonstrated that i) phosphorylation of Rb at Ser795 is significantly reduced in senescent BRCA1^{mut/+} HMECs (FIG. 5); ii) E2F expression and cyclin A (mRNA and protein) are both decreased in senescent BRCA1^{mut/+} HMECs (FIG. 5); iii) SIRT1 expression is reduced in BRCA1^{mut/+} HMECs (FIG. 6); iv) inhibition of SIRT1 in WT HMECs leads to premature senescence (FIG. 6); and v) BRCA1^{mut/+} HMECs exhibit genomic instability and rapid telomere erosion. Decreased levels of SIRT1 in BRCA1-mutant HMECs, but not fibroblasts, therefore, lead to a reduction in deacetylated Rb, thereby prompting senescence. Reduction of SIRT1 activity in HMECs, but not fibroblasts, also promotes genomic instability by reduction in chromosomal and/or telomeric deacetylation.

### SIRT1 Deacetylase Activity.

To assess the effect of SIRT1 deacetylase activity on premature senescence in HMECs, lentiviral infection followed by puromycin-resistance selection is used to generate cells that stably express SIRT1 shRNA or scrambled shRNA. To characterize the effect of SIRT1 knockdown, population doublings of the cells are recorded and SA-β-gal assay is performed when cells undergo senescence. Samples are also collected for the analysis of acetylated-, phosphorylated-, and total-Rb levels by Western blotting, and corroborated with the assessment of the E2F target genes such as cyclin A using qRT-PCR. These data recapitulate the observations made in senescent BRCA1^{mut/+} HMECs.

To examine whether increase in SIRT1 levels is sufficient to overcome premature senescence in HMECs, SIRT1 or enzymatically dead SIRT1 is overexpressed in BRCA1 mut/+HMECs. Cell lines stably expressing WT or mutant SIRT1, or an empty vector control, are generated. The effect of SIRT1 overexpression on cell proliferation is assessed by counting the population doublings and staining for SA-β-gal. Samples are collected to perform a similar analysis of Rb and cyclin A levels. SIRT1 overexpression can rescue the phenotype of BRCA1^{mut/+} HMECs. Pharmacological activators of SIRT1, e.g., resveratrol, are also used to assess the effect of SIRT1 activity on rescue from HIS.

### SIRT1 Activity Effects Genomic Stability.

Global histone H3 and H4 modifications in BRCA1^{mut/+} HMECs with or without SIRT1 are examined be determining global levels of acetylated H3 and H4 (Nakahata, Y. et al., Cell, 134:329-40, 2008) in pre-senescent and senescent BRCA1^{mut/+} and WT HMECs, as well as cells in which SIRT1 has been inhibited using shRNA or in which SIRT1 has been expressed.

Chromatin immunoprecipitation (ChIP): ChIP studies are performed (Garcia-Cao, M. et al., Nat. Genet., 36:94-99, 2004) for total H3 as well as specifically modified forms of acetylated H3: H3K9Ac, H3K4Ac, and H3K12Ac and acetylated H4 at telomeric DNA in pre-senescent, and senescent BRCA1^{mut/+} HMECs. A telomeric probe containing TTAGGG repeats or a probe recognizing major satellite sequences, which is characteristic of pericentric heterochromatin, is used. The amount of telomeric and pericentric DNA after ChIP is normalized for the total telomeric or centromeric DNA signal, respectively, for each genotype and for the H3 and H4 abundance at these regions, thus correcting for differences in the number of telomere repeats or in nucleosome spacing. To determine if histone modification at telomeres is be mediated by SIRT1 activity, histone acetylation in WT HMECs following SIRT1 knockdown is examined.

### Example 2

Human mammary epithelial cells (HMECs), but not other cell types from individuals harboring deleterious mutations in BRCA1 (BRCA1^{mut/+}) exhibit increased genomic instability and rapid telomere erosion in the absence of tumor suppressor loss. Furthermore, a novel form of HIS specific to epithelial cells is identified, which is triggered by pRb pathway activation rather than p53 induction. HIS and rapid telomere erosion in HMECs is mediated by misregulation of the NAD⁺-dependent deacetylase SIRT1 that leads to increased levels of acetylated pRb as well as H4K16-Ac both globally and at telomeric regions. These results identify a novel form of cellular senescence and provide a molecular basis for the cell and tissue specific predisposition of breast cancer development associated with BRCA1 haploinsufficiency.

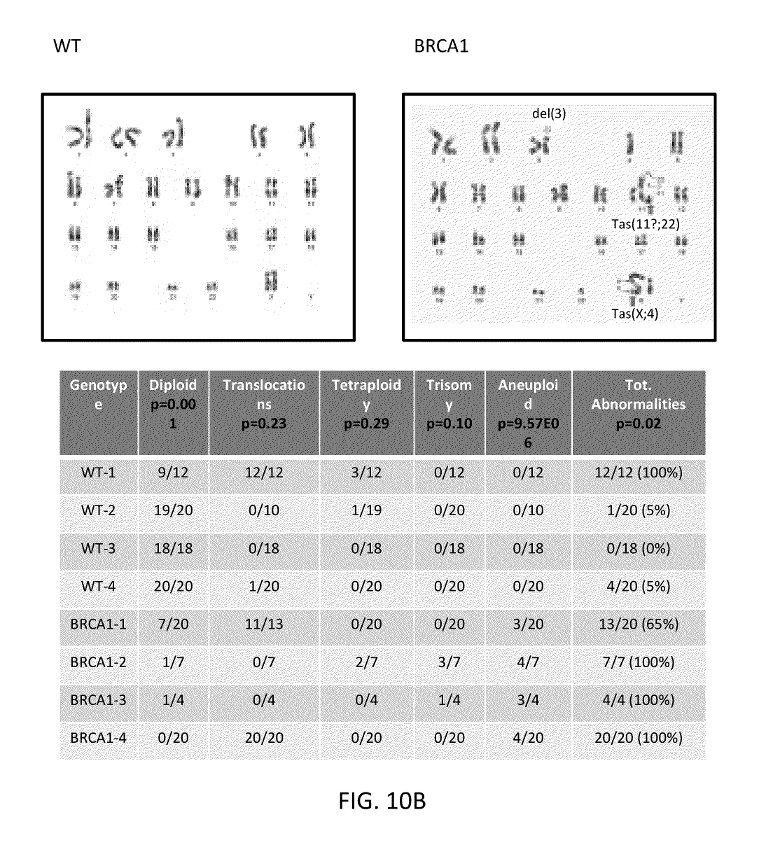

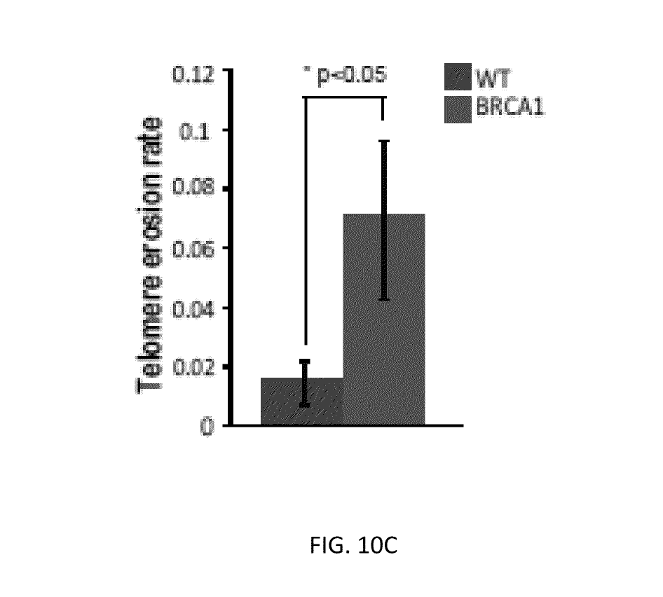

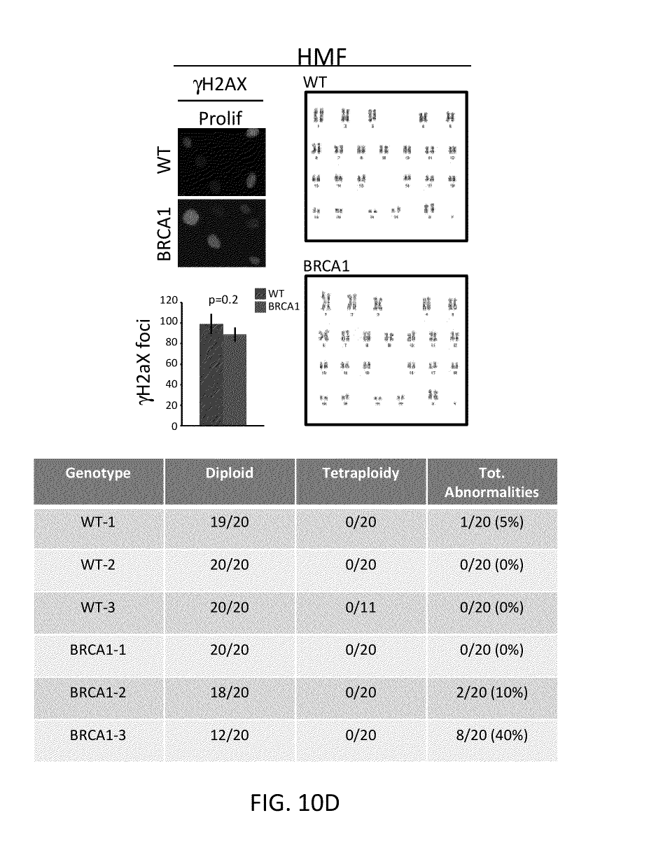

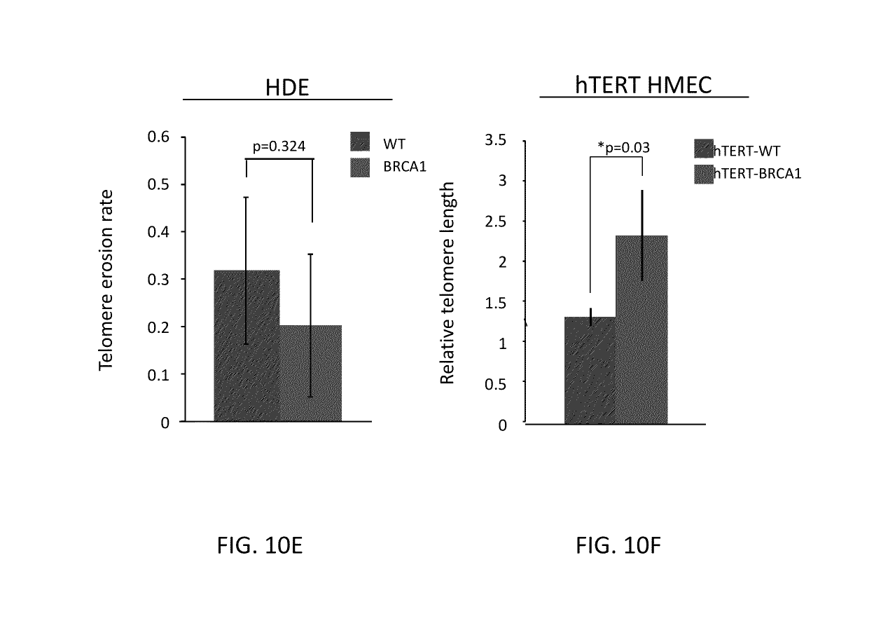

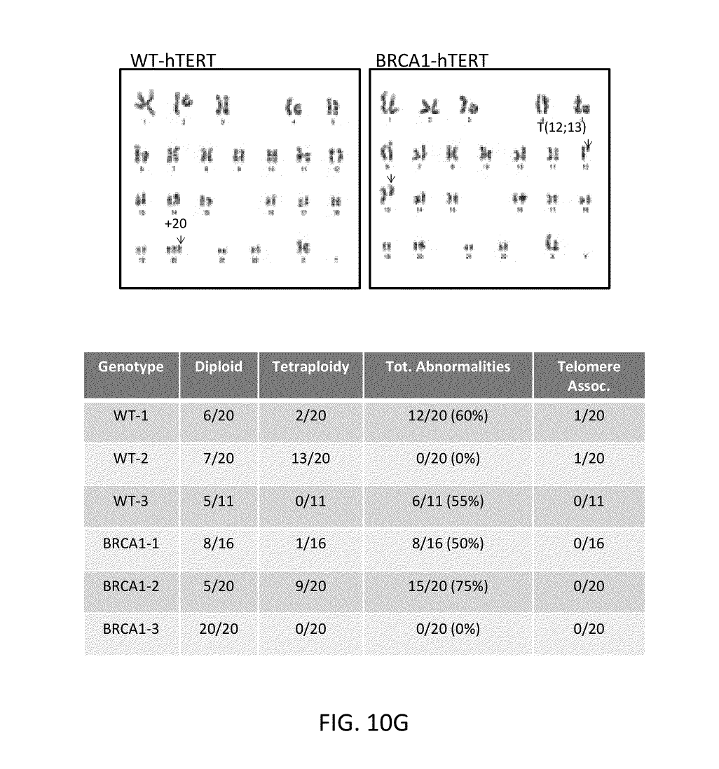

BRCA1 is involved in many processes essential for genomic maintenance and its deficiency causes abnormalities in homologous recombination, double strand break repair, S-phase, G2/M, and spindle checkpoints and in centrosomal regulation (Zhang, J. & Powell, S., Mol. Cancer. Res., 3:531-9, 2005). Although BRCA1-deficiency has been shown to directly lead to genomic instability and increased risk of neoplastic transformation in many cell types, accumulating evidence indicates that loss of BRCA1 in the breast is a late and stochastic event during tumor progression, affecting the mutant or wild-type alleles at similar frequencies (Martins, F. et al., Cancer Discov., 2:503-11, 2012; Clarke, C. et al., Br. J. Cancer, 95:515-9, 2006). Studies have suggested that BRCA1-haploinsufficiency rather than total BRCA1 loss might be associated with increased genomic instability and inefficient DNA damage repair (Baldeyron, C. et al., Oncogene, 21:1401-10, 2002; Rennstam, K. et al., Genes Chromosomes Cancer, 49:78-90, 2010). To explore this in a cell-type and tissue-specific context, the growth properties of primary HMECs, primary dermal epithelial cells (HDEs) and fibroblasts isolated from disease-free breast (RMF) and skin (HDF) tissues of women with or without deleterious mutations in BRCA1, (BRCA1^{mut/+}, WT, respectively; Table 1) were examined. Proliferating BRCA1^{mut/+} HMECs exhibited significantly higher levels of phosphorylated ATM/ATR substrates as well as γH2AX and 53BP1 recruitment to DNA indicating that these cells suffer increased DNA damage and double-strand breaks compared to proliferating WT cells (p=0.01; p=0.03; p=0.009, respectively; FIG. 10A). This was observed across multiple patient-derived BRCA1^{mut/+} HMECs and across multiple BRCA1 mutations (Table 1). Furthermore, proliferating cultures of BRCA1^{mut/+} HMECs displayed increased numbers of chromosomal abnormalities including unbalanced translocations, telomeric fusions and aneuploidy (FIG. 10B).

TABLE 1
BRCA1 mutation status

| | | Patient | Mutation | | |
|---|---|---|---|---|---|
| Sample ID | Tissue Type | Age | (y/n) | Specific Mutation | Analysis |
| 616 | Proph.Mastec-Brca1 | 40 | yes | exon13ins | qFISH |
| 617 | Proph.Mastec-Brca1 | 53 | yes | BRCA1 5385insC | qFISH |
| 627 | Proph.Mastec-Brca1 | 53 | yes | BRCA1 187 delAG | qFISH |
| 628 | Proph.Mastec-Brca1 | 40 | yes | BRCA1 5385 insC | qFISH |
| 629 | Proph.Mastec-Brca1 | 44 | yes | BRCA1 2800 delAA | qFISH, LOH, GC, P/R, |
| | | | | | TELPCR, MET |
| 634 | Proph.Mastec-Brca1 | 39 | yes | BRCA14184del4 | qFISH, LOH, TELPCR, |
| | | | | | MET |
| 635 | Proph.Mastec-Brca1 | 35 | yes | BRCA1 5385 insC | qFISH, LOH, GC, P/R, |
| | | | | | TELPCR, MET |
| 642 | Proph.Mastec-Brca1 | 36 | yes | BRCA1 187 delAG | qFISH, LOH, GC, P/R, |
| | | | | | TELPCR, MET |
| 643 | Proph.Mastec-Brca1 | 47 | yes | BRCA1C61G | qFISH |
| 650 | Proph.Mastec-Brca1 Skin | 33 | yes | BRCA1 943ins10 | LOH, GC, P/R, TELPCR |
| 651 | Proph.Mastec-Brca1 Skin | 47 | yes | delexon14intron14 | LOH, GC, P/R, TELPCR |
| 652 | Proph.Mastec-Brca1 Skin | 46 | yes | BRCA1 4154delA | LOH, GC, P/R, TELPCR |

qFISH = in vivo telomere length; LOH = LOH analysis; GC = growth curves; P/R = protein and RNA analysis; TELPCR = telomere length PCR; MET = metaphase spreads

Given the increase in genomic instability, particularly in lesions associated with telomere dysfunction, the question of whether telomere attrition might be responsible for the increased chromosomal alterations in BRCA1^{mut/+} HMECs was addressed. Indeed, there was a −4-fold increase in telomere erosion rates in BRCA1^{mut/+} HMECs as they approached cell cycle arrest compared to telomere erosion rates in WT cells (p<0.04; FIG. 10C). Consistent with the findings that BRCA1^{mut/+} HMECs exhibit increased DDR, genomic instability and telomere dysfunction, gene set enrichment analysis (GSEA, Table 2) of global transcriptional profiling data collected from proliferating primary BRCA1^{mut/+} HMECs (N=6) compared to age-matched primary WT HMECs (N=6) revealed a significant enrichment of genes associated with DNA repair (p<0.0137), homologous recombination (p<0.022), as well as genes involved in activation of ATR in response to replicative stress (p<0.049), and extension of telomeres (p<0.049).

TABLE 2
GSEA analysis

| Name of the | Gene Set | | | | |
|---|---|---|---|---|---|
| pathway | SIZE | ES | NES | NOM p-val | FDR q-val |
| REACTOME CELL | 261 | −0.18221466 | −3.333048 | 0 | 0 |
| CYCLE MITOTIC | | | | | |
| REACTOME E2F | 27 | −0.49427503 | −3.0600283 | 0 | 2.95E−04 |
| MEDIATED | | | | | |
| REGULATION OF DNA | | | | | |
| REPLICATION | | | | | |
| REACTOME G2 M | 72 | −0.27993816 | −2.8140864 | 0 | 0.00105462 |
| TRANSITION | | | | | |
| REACTOME | 33 | −0.40297702 | −2.7753196 | 0 | 0.0012494 |
| ACTIVATION OF ATR | | | | | |
| IN RESPONSE TO | | | | | |
| REPLICATION STRESS | | | | | |
| REACTOME | 60 | −0.29642725 | −2.7085989 | 0 | 0.00188686 |
| CENTROSOME | | | | | |
| MATURATION | | | | | |
| REACTOME E2F | 18 | −0.51949006 | −2.6574273 | 0 | 0.00293562 |
| TRANSCRIPTIONAL | | | | | |
| TARGETS AT G1 S | | | | | |
| REACTOME G2 M | 38 | −0.35594466 | −2.5365891 | 0 | 0.00567763 |
| CHECKPOINTS | | | | | |
| REACTOME | 27 | −0.3674328 | −2.2960405 | 0 | 0.01802512 |
| ACTIVATION OF THE | | | | | |
| PRE REPLICATIVE | | | | | |
| COMPLEX | | | | | |
| REACTOME MITOTIC | 71 | −0.23039581 | −2.2924228 | 0 | 0.01653609 |
| PROMETAPHASE | | | | | |
| BIOCARTA MCM | 18 | −0.44952336 | −2.2790813 | 0 | 0.01604816 |
| PATHWAY | | | | | |
| KEGG CELL CYCLE | 111 | −0.17238668 | −2.2049823 | 0 | 0.02107831 |
| REACTOME MITOTIC | 134 | −0.15738487 | −2.1053176 | 0 | 0.03355169 |
| M M G1 PHASES | | | | | |
| REACTOME G1 S | 94 | −0.18183643 | −2.066683 | 0 | 0.03683207 |
| TRANSITION | | | | | |
| CHICAS RB1 TARGETS | 221 | 0.27887425 | 4.7940974 | 0 | 0 |
| GROWING | | | | | |
| CHICAS RB1 TARGETS | 495 | 0.16796239 | 4.2921963 | 0 | 0 |
| SENESCENT | | | | | |
| KAMMINGA EZH2 | 39 | 0.41060108 | 3.059856 | 0 | 0 |
| TARGETS | | | | | |
| V$E2F1 Q3 | 210 | 0.15255454 | 2.5253246 | 0 | 2.13E−04 |
| TANG SENESCENCE | 52 | 0.2647142 | 2.2747254 | 0 | 0.00351118 |
| TP53 TARGETS DN | | | | | |
| CHICAS RB1 TARGETS | 490 | 0.082020804 | 2.0581293 | 0.00194932 | 0.01106058 |
| CONFLUENT | | | | | |
| KUMAMOTO | 10 | 0.5047875 | 1.9616181 | 0.0020202 | 0.01498721 |
| RESPONSE TO | | | | | |
| NUTLIN 3A DN | | | | | |
| REACTOME DNA | 27 | −0.29543674 | −1.8379503 | 0.00567108 | 0.08739737 |
| STRAND | | | | | |
| ELONGATION | | | | | |
| REACTOME DNA | 93 | −0.1684125 | −1.8715832 | 0.01372549 | 0.07727332 |
| REPAIR | | | | | |
| REACTOME DNA | 72 | −0.17291869 | −1.751833 | 0.01724138 | 0.1213078 |
| REPLICATION PRE | | | | | |
| INITIATION | | | | | |
| KEGG HOMOLOGOUS | 27 | −0.27458984 | −1.6734208 | 0.02249489 | 0.15364626 |
| RECOMBINATION | | | | | |
| REACTOME | 84 | −0.16309172 | −1.7996148 | 0.02574257 | 0.09985348 |
| SYNTHESIS OF DNA | | | | | |
| REACTOME M G1 | 59 | −0.18244392 | −1.6782709 | 0.0308642 | 0.15378681 |
| TRANSITION | | | | | |
| REACTOME CELL | 99 | −0.14401378 | −1.663237 | 0.03571429 | 0.15834156 |
| CYCLE CHECKPOINTS | | | | | |
| CHICAS RB1 TARGETS | 76 | 0.16329645 | 1.6577407 | 0.04268293 | 0.07548876 |
| LOW SERUM | | | | | |
| REACTOME | 24 | −0.2721803 | −1.5520241 | 0.04930966 | 0.23302579 |
| EXTENSION OF | | | | | |
| TELOMERES | | | | | |

To determine whether increased DDR and genomic instability are features of BRCA1 haploinsufficiency in general, DDR, karyotypes and telomere erosion rates in proliferating RMFs and HDEs from age-matched individuals were examined, respectively. In contrast to HMECs, there was no difference in DDR and chromosomal abnormalities between proliferating WT and BRCA1^{mut/+} mammary fibroblasts (FIG. 10D), nor did HDEs exhibit a difference in telomere attrition rates (p=0.324; FIG. 10E).

Given these findings, another question addressed was whether expression of hTERT rescues telomere erosion and genomic instability in BRCA1^{mut/+} HMECs. Indeed, overexpression of the catalytic subunit of telomerase (hTERT) in either WT or BRCA1^{mut/+} cells resulted in cellular immortalization. BRCA1^{mut/+} HMECs, however, exhibited telomeres that were 2-fold longer than WT cells, and chromosomal abnormalities associated with telomere erosion were attenuated in hTERT expressing BRCA1^{mut/+} HMECs. These results indicate that hTERT expression is able to alleviate telomere dysfunction and telomere-associated genomic instability in BRCA1^{mut/+} HMECs (FIGS. 10F and 10G).

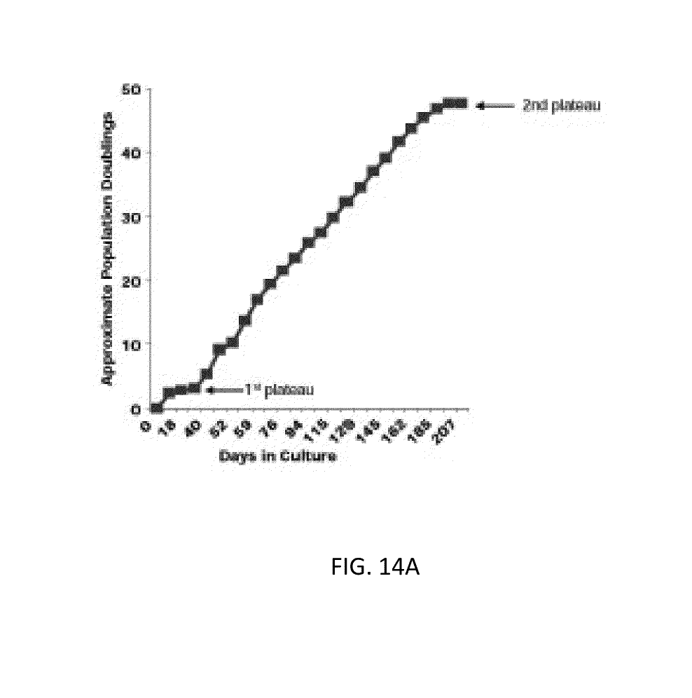

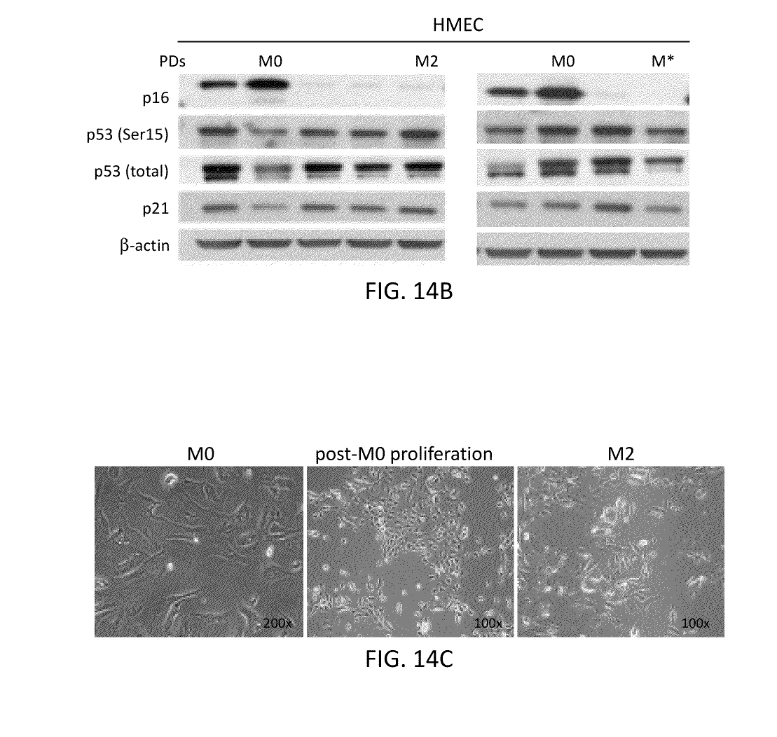

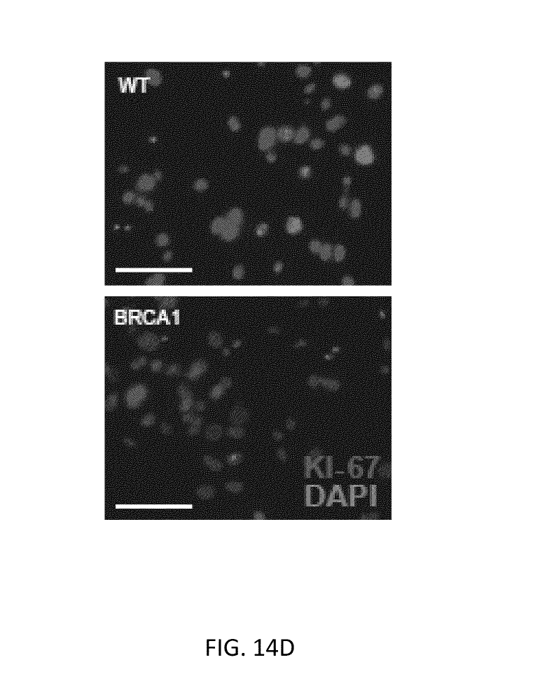

Cellular senescence has emerged as an intrinsic mechanism to suppress cellular proliferation and neoplastic transformation in the context of many forms of stress including telomere erosion, oncogene activation, and most recently tumor suppressor loss. Therefore, the question was addressed as to whether the increased telomere erosion and dysfunction in BRCA1^{mut/+} HMECs leads to premature growth arrest. WT HMECs encounter two mechanistically distinct senescent-like barriers in vitro (FIG. 14). The first proliferative barrier referred to as stasis or M0, is associated with classical p16/INK4a-dependent stress induced senescence and concomitant p53 pathway activation (FIG. 14; Garbe, J. et al., Cell Cycle, 6:1927-36, 2007). Cells that emerge from this barrier do so through down-regulation of p16/INK4a and rapidly proliferate until they reach a second senescence barrier referred to as agonescence/M2 (FIG. 14). Unlike senescence, agonescence/M2 is induced through p53 pathway activation in response to DNA damage and genomic instability as a consequence of telomere attrition and dysfunction. In addition, the apparent proliferative arrest observed during agonescence/M2 is maintained through a balance of proliferation and apoptosis.

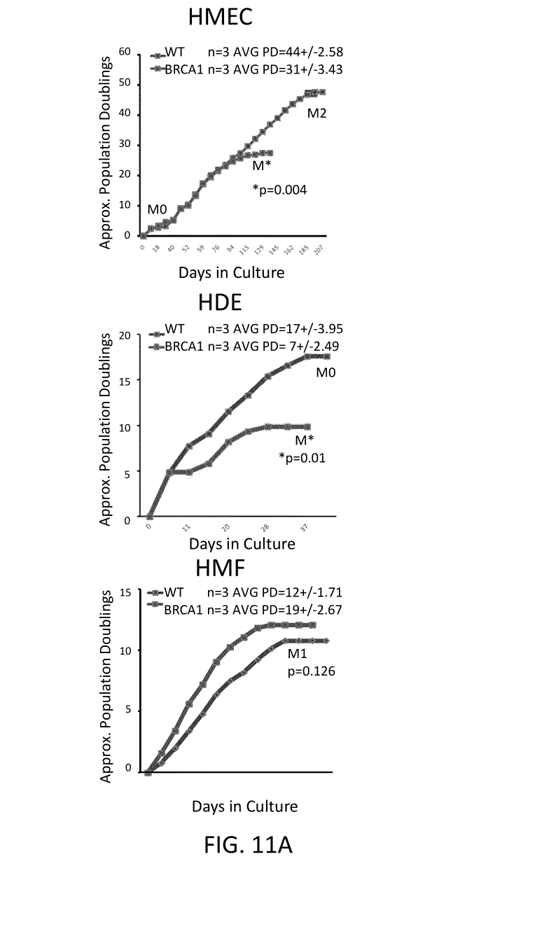

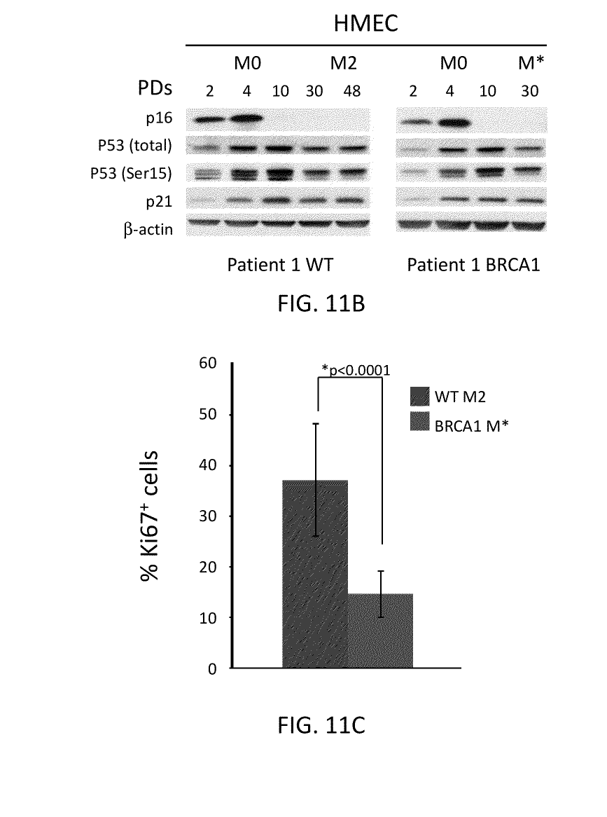

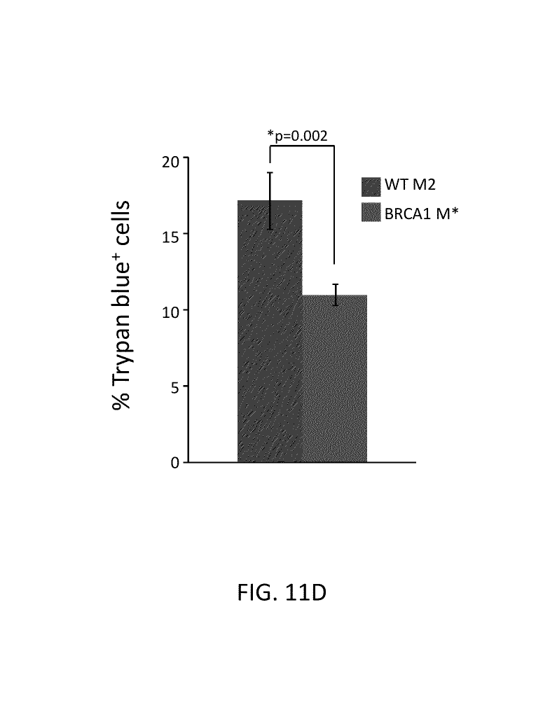

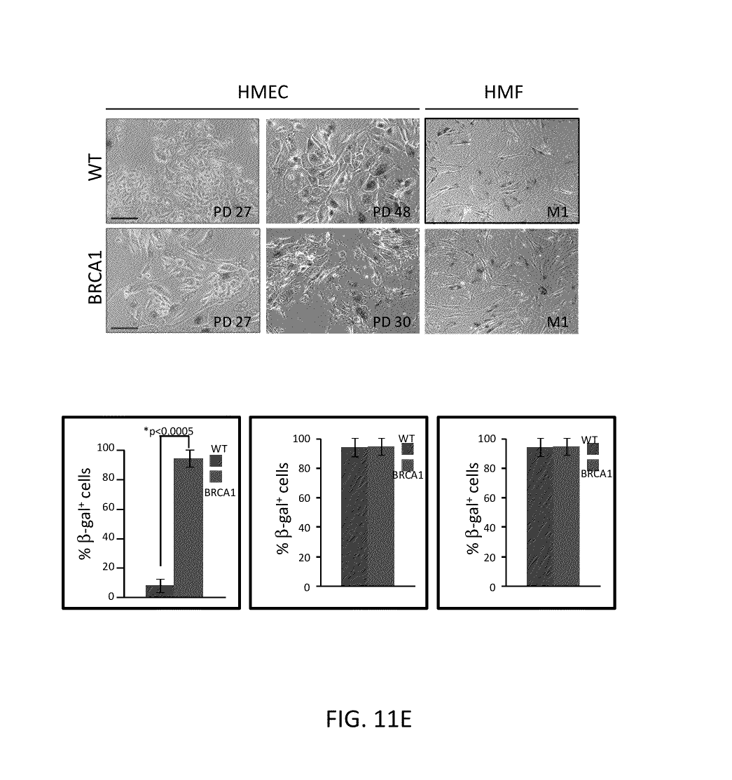

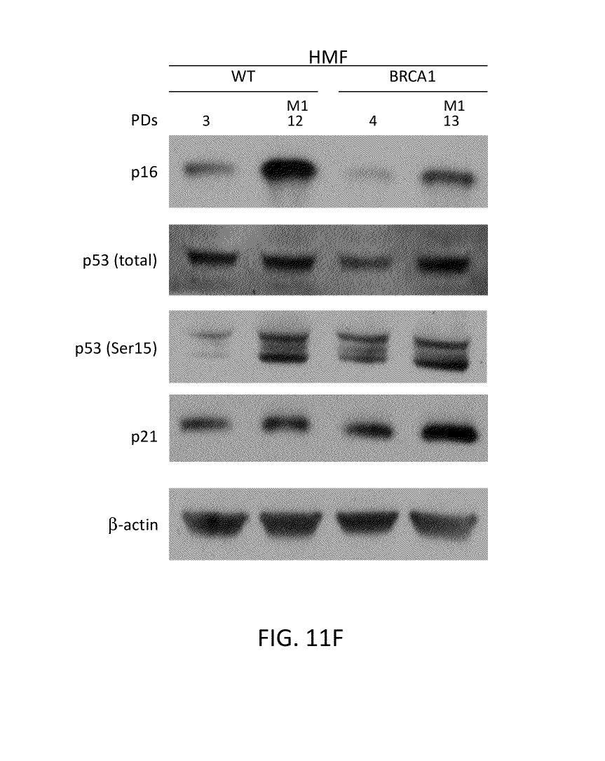

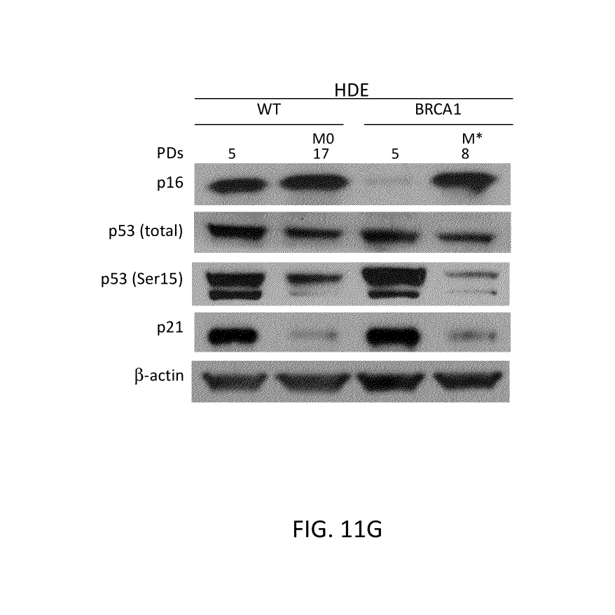

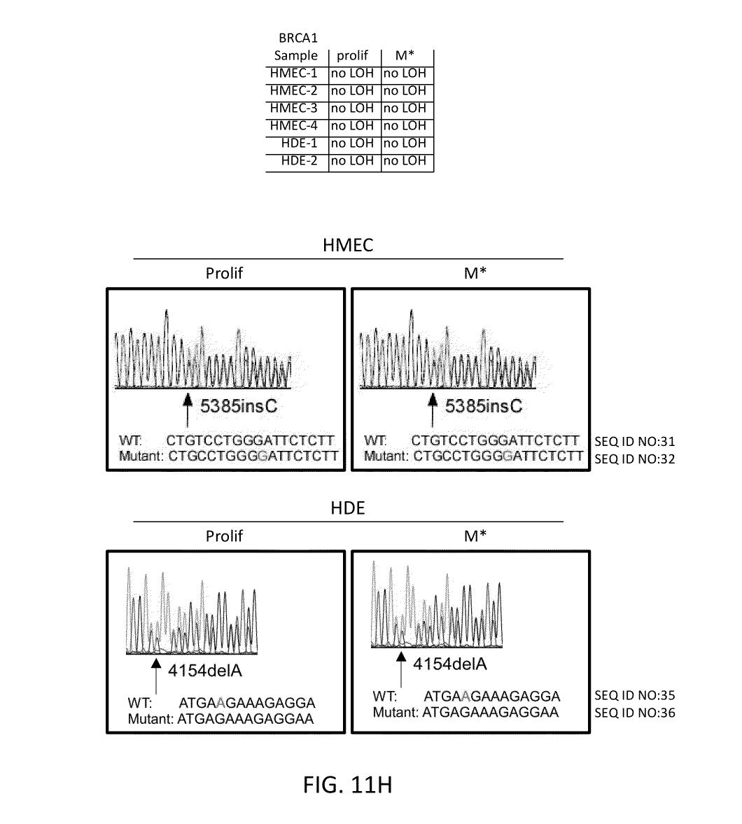

Examination of BRCA1^{mut/+} and WT HMECs revealed similar growth kinetics and molecular responses in early cultures; both WT and BRCA1^{mut/+} HMECs entered into M0/stasis, induced p16/INK4a, and p53 protein expression in a similar fashion (FIGS. 11A, 11B and 14B). Likewise, WT and BRCA1^{mut/+} HMECs overcame M0 with similar frequencies and efficiencies, and both exhibited loss of p16/INK4a expression upon bypass of stasis (FIGS. 11A, 11B and 14B). However, while WT HMECs on average continued to proliferate for an additional ˜44 PDs, BRCA1^{mut/+} HMECs stopped proliferating after −31 PDs (FIG. 11A). This premature growth arrest (M*) was observed across multiple patient-derived BRCA1^{mut/+} HMECs with different BRCA1 mutations and was observed in BRCA1^{mut/+} HMECs well before agonescence/M2 in WT HMECs (p=0.004, Table 1). Unlike M2, M* differed in that the proliferation and apoptosis indexes were significantly lower indicating cell cycle arrest in BRCA1^{mut/+} HMECs (FIGS. 11C and 11D; p<0.0001, p=0.002, respectively; FIG. 14D). However, M* and M2 were both associated with the senescent phenotype characterized by enlarged, flattened morphology and positive staining for SA-β-gal (FIG. 11E).

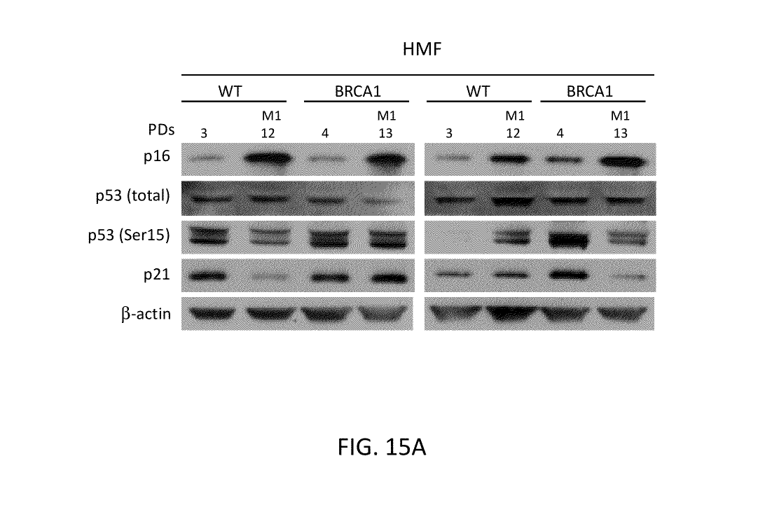

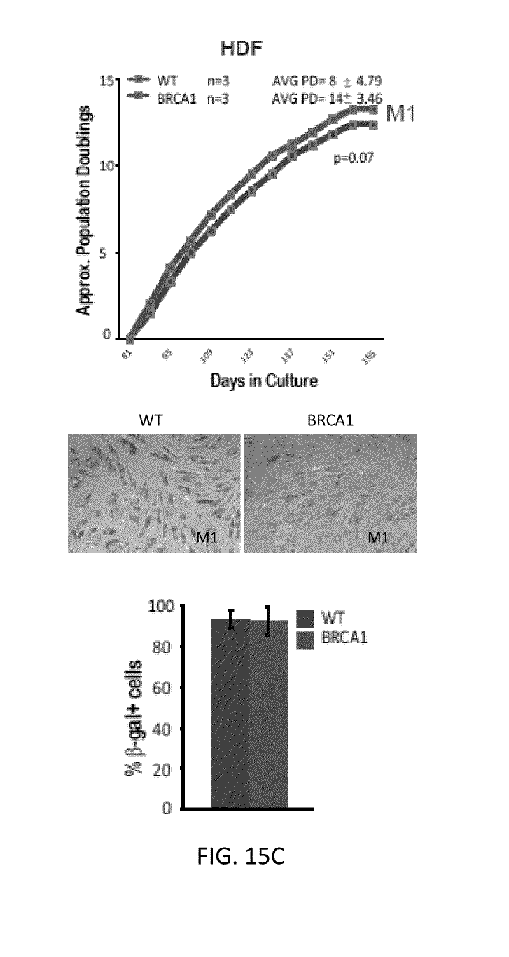

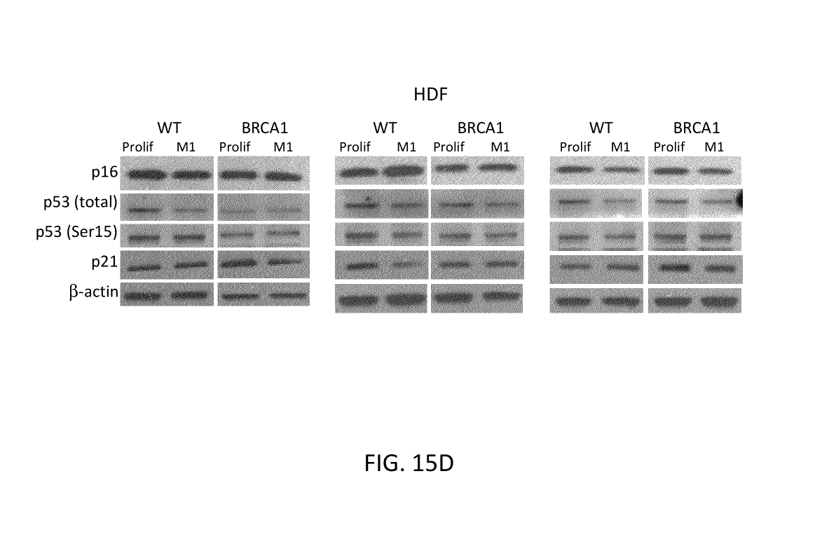

To determine whether premature senescence was a feature of BRCA1 haploinsufficiency in other cell and tissue types, the growth kinetics as well as p53 and p16 pathway activation in fibroblasts and skin epithelial cells from age-matched individuals was examined. Similar to HMECs, BRCA1^{mut/+} HDEs also underwent rapid premature growth arrest with typical features of senescence compared to WT HDEs (Avg. PD=7±2.5 vs. Avg. PD=17±4, respectively; FIG. 11A). This was also observed across multiple patient-derived BRCA1^{mut/+} samples and across multiple BRCA1 mutations (p=0.01, Table 1). In contrast to epithelial cells, BRCA1^{mut/+} mammary and skin fibroblasts underwent similar population doublings in culture compared to WT cells, after which cells became SA-β-gal positive and stopped dividing (FIGS. 11A, 11E and 15D). In addition, consistent with classical replicative senescence M1, skin and mammary fibroblasts induced p16/INK4a as they approached senescence (FIGS. 11F, 11G and 15D).

Several lines of evidence have shown that LOH of tumor suppressor genes (e.g., VHL, PTEN or NF1) can lead to the induction of premature senescence programs. Examination of BRCA1^{mut/+} HMECs and HDEs for LOH, however, revealed that both WT and mutant alleles were present in both proliferative and senescent cells (FIG. 11H). These findings indicate that loss of the remaining WT allele was not responsible for premature senescence and that haploinsufficiency for BRCA1 results in the engagement of a novel premature senescence-like barrier in epithelial cells but not other cell types (HIS).

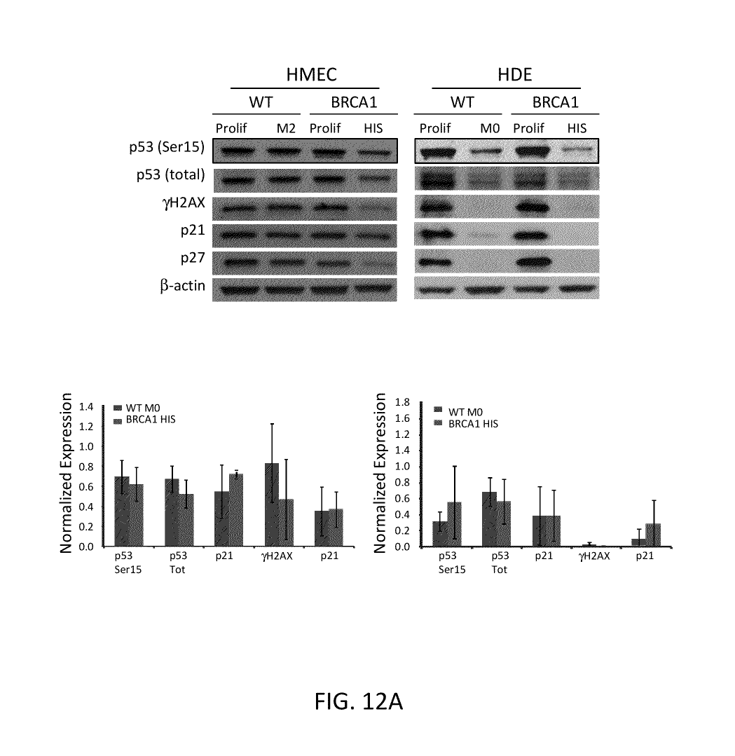

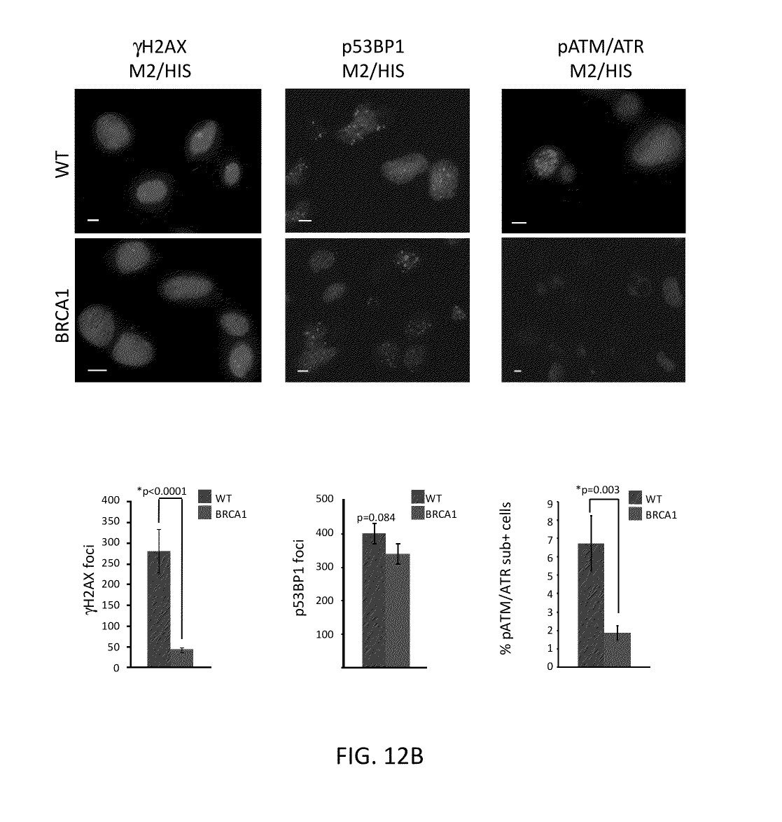

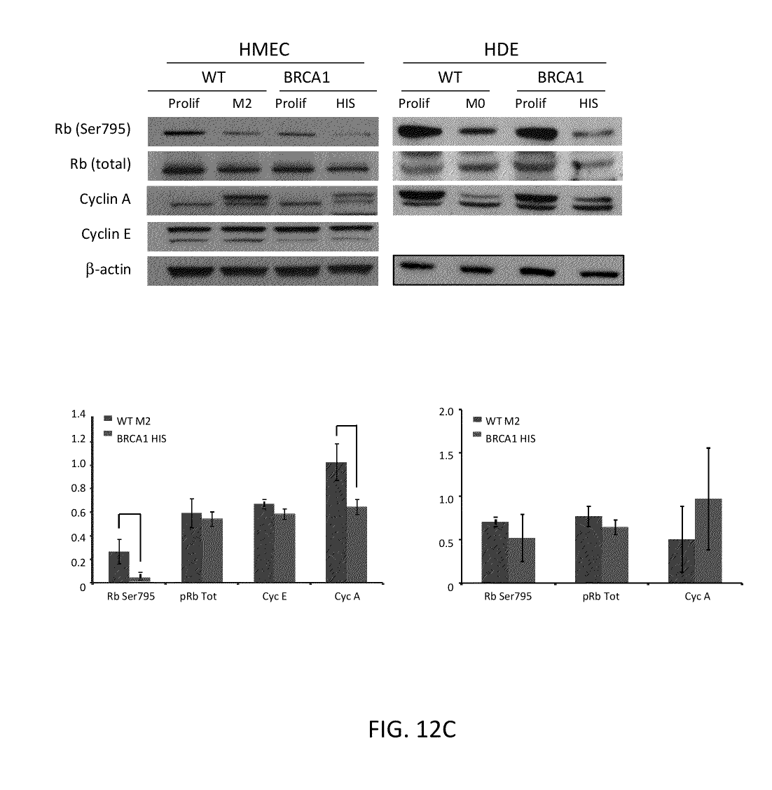

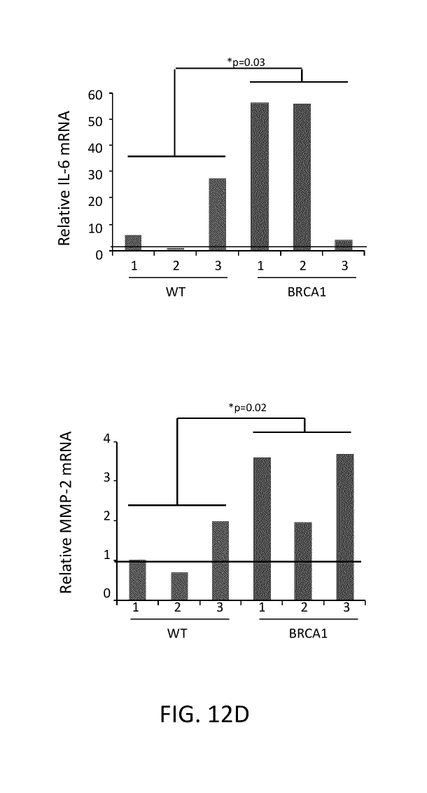

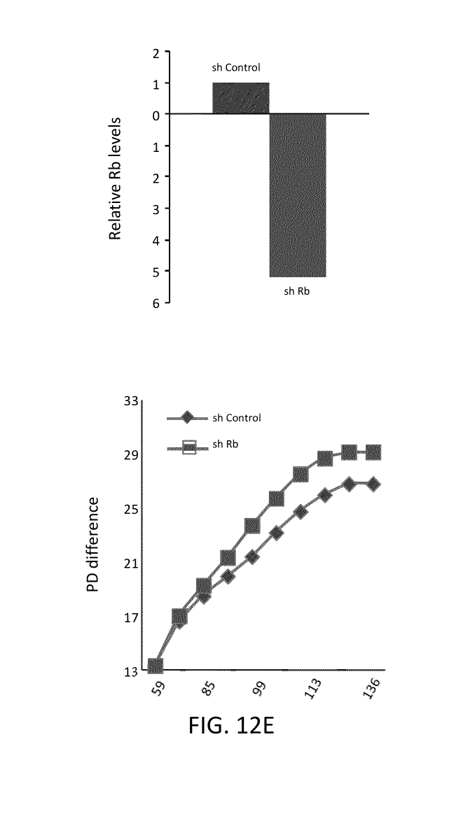

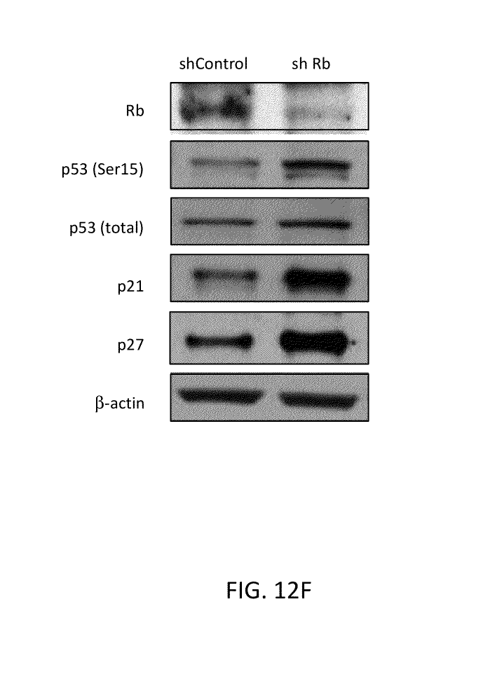

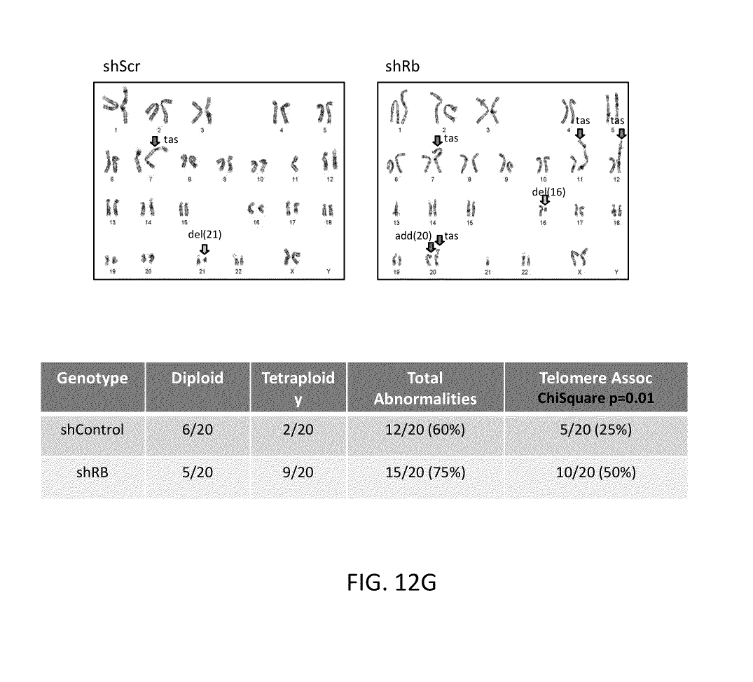

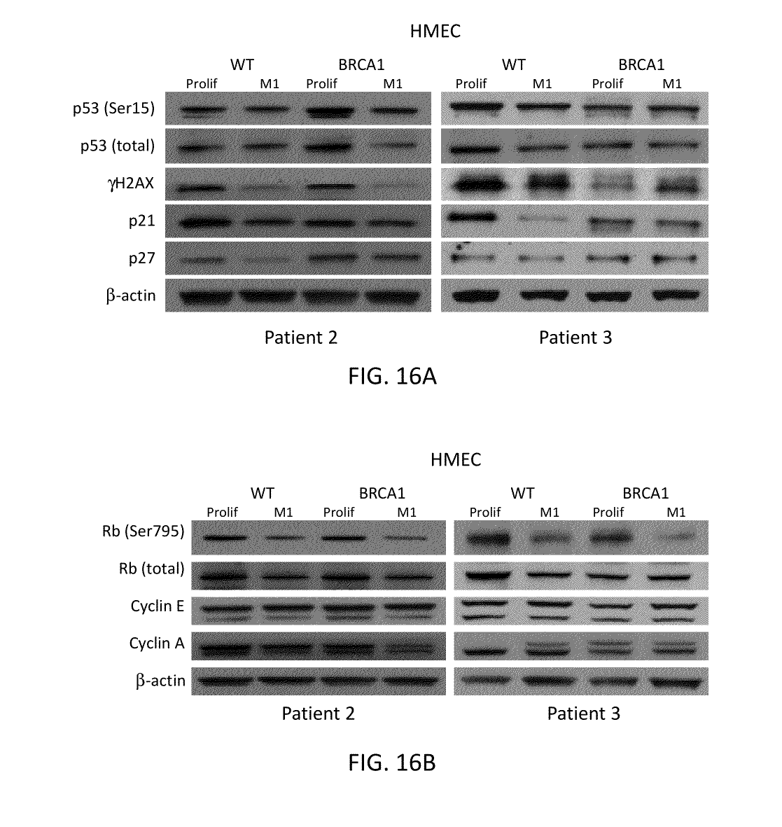

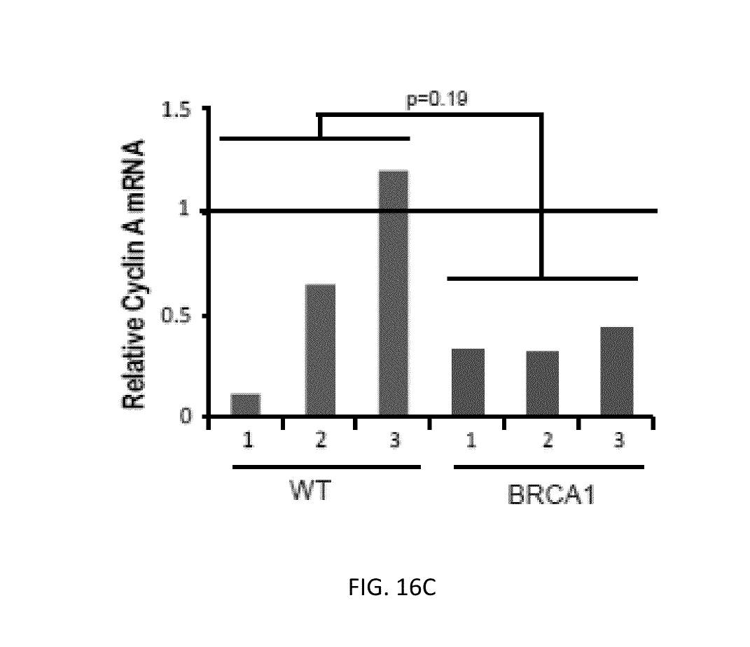

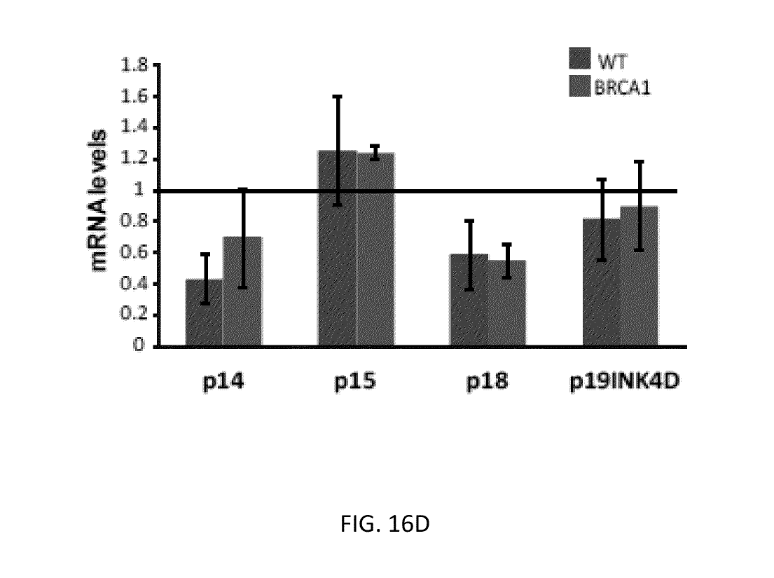

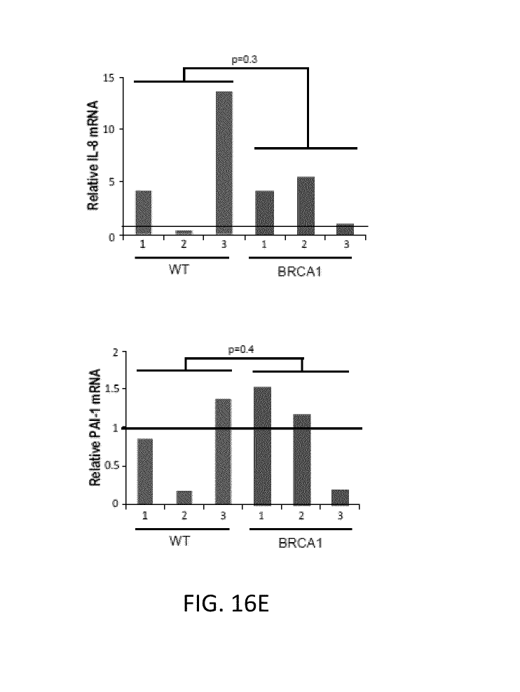

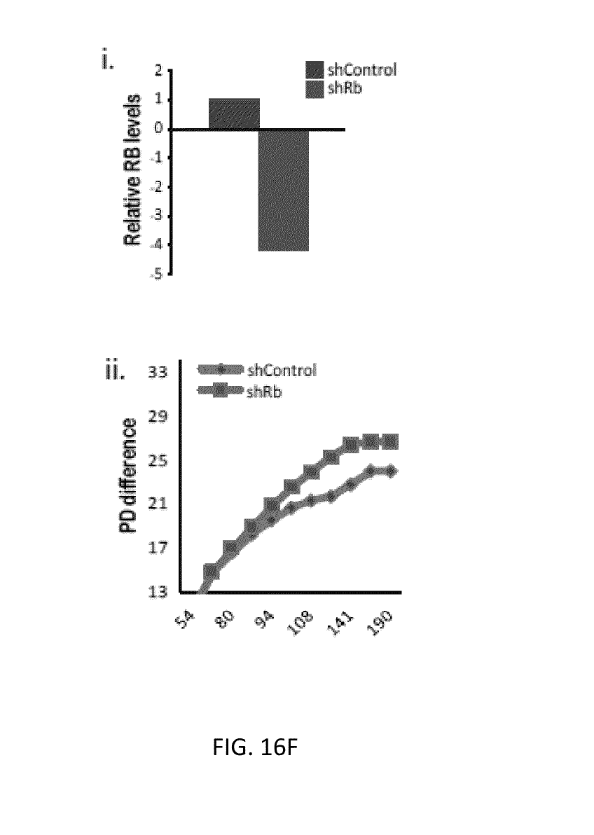

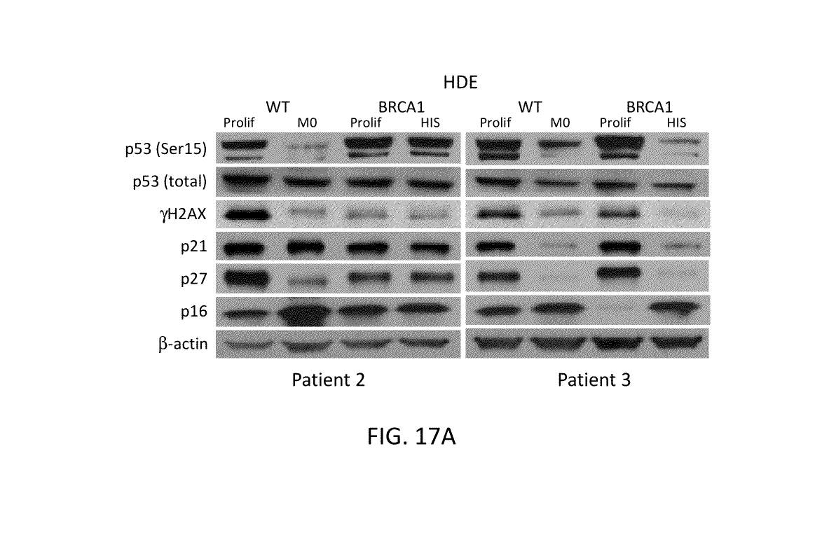

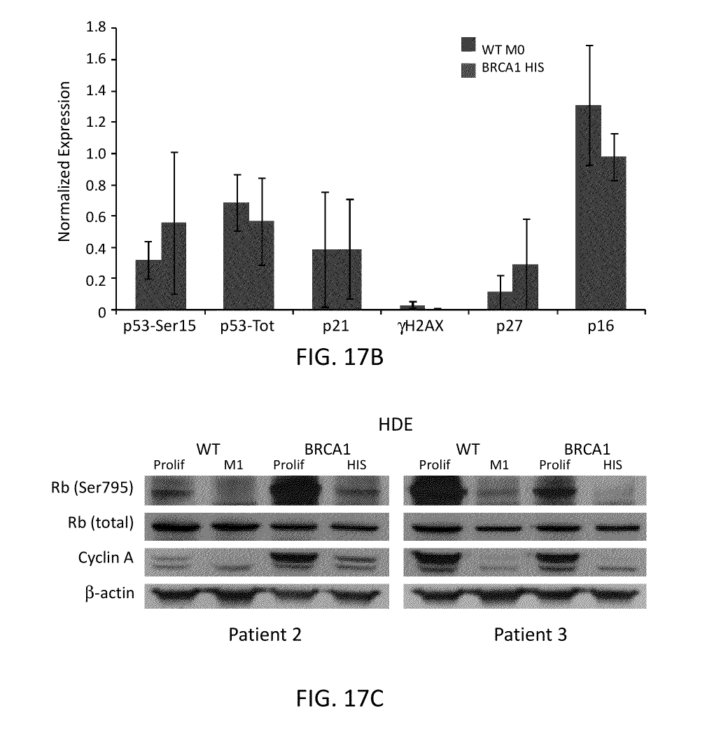

While premature senescence in BRCA1-heterozygous cells has not been previously reported, senescence in BRCA1-deficient mouse embryonic fibroblasts or human cells has been reported to be triggered in response to excessive DDR through a p53-dependent pathway (Tu, Z. et al., Dev. Cell, 21:1077-91, 2011). In contrast to this process however, the levels of critical components of DDR and p53 pathway activation, such as phosphorylated p53 (Ser15), total p53, p21, p27 as well as phosphorylated ATM/ATR substrates, γH2AX and 53BP1 were not elevated in senescent BRCA1^{mut/+} HMECs or HDEs indicating that there was no preferential induction of the p53 pathway in BRCA1-heterozygous cells leading to HIS (FIGS. 12A, 12B, 16 and 17). Rather, HIS in BRCA1^{mut/+} HMECs actually showed a significant reduction in the number of cells with phosphorylated ATM/ATR substrates (p=0.003; FIG. 12B), and a reduction in γH2AX foci (p<0.0001; FIG. 12B) when compared to agonescent WT HMECs suggesting that engagement of the DDR at HIS is suppressed and differs from that in M2 (FIGS. 11A, 11B and 16).

Since senescence is mediated by activation of the pRb pathway, the levels of pRb phosphorylation, and the E2F target genes, cyclin A and cyclin E were assessed in HMECs and HDEs. Although total levels of pRb were similar, levels of phosphorylated pRb at Ser795 were reduced in senescent BRCA1^{mut/+} HMECs compared to WT HMECs (FIGS. 12C, 16 and 17). In addition, levels of the E2F target gene cyclin A were significantly decreased in senescent BRCA1^{mut/+} HMECs compared to WT HMECs (FIGS. 12C and 16). Consistent with these findings, GSEA of gene expression data analyzed from BRCA1^{mut/+} HMECs revealed a significant enrichment of various pRb target genes including those associated with senescence (p<10⁴; Table 2), E2F1-regulated genes (p<10⁴; Table 2) as well as genes down-regulated in senescent cells lacking p53 activity (p<10⁴; Table 2). Furthermore, examination of levels of senescence-associated secreted factors (SASFs) such as IL-6, IL-8, MMP-2 and PAI-1 revealed that IL-6 and MMP-2 (but not IL-8 or PAI-1) were increased in senescent BRCA1^{mut/+} HMECs compared to M2 WT HMECs (FIGS. 11D and 16E). In addition, unlike pRb-induced senescence in other contexts, levels of cell cycle inhibitors including p14/ARF, p15/INK4b, p18/INK4c and p191NK4d did not differ between WT or BRCA1^{mut/+} HMECs (FIG. 16).

Using hairpin-mediated lentiviral knockdown of pRb in BRCA1^{mut/+} HMECs, the question of whether senescence in BRCA1^{mut/+} HMECs is in fact induced by pRb was examined. Compared to control BRCA1^{mut/+} HMECs, knockdown of pRb led to an increase in replicative potential (FIGS. 12E and 16F), indicating that pRb is one of the main mediators of premature senescence. Interestingly, BRCA1^{mut/+} HMECs forced to proliferate as a result of pRb knockdown eventually arrested (FIG. 12E). Moreover, this bypass of HIS led to a further significant increase in telomeric fusions and genomic instability (p=0.01), with concomitant p53 pathway activation (FIG. 12F, G).

Given that there were no differences in the expression levels of cell cycle inhibitors in BRCA1^{mut/+} HMECs despite the reduction in pRb phosphorylation and activity, it was possible that an alternate mechanism was responsible for pRb activation in these cells. Indeed, pRb phosphorylation on multiple residues can be regulated by acetylation events that are catalyzed by the NAD-dependent deacetylase SIRT1 in pRb-SIRT1 complexes. Some studies have shown that SIRT1 protein expression decreases during replicative senescence and that there is a negative correlation between levels of SIRT1 and SA-β-gal activity. Moreover, cell cycle arrest in these settings was shown to be associated with both decreased pRb phosphorylation and increased pRb acetylation. In addition, SIRT1 has also been reported to mediate deacetylation of histone H3K9, H3K56 and H4K16 during cellular aging on telomeric and subtelomeric regions, thereby leading to loss of histones, shorter telomeres and genomic instability (Dang, W. et al., Nature, 459:802-7, 2009). Thus, misregulation of SIRT1 in BRCA1^{mut/+} HMECs results in both modifications of pRb acetylation leading to induction of HIS, as well as changes in histone acetylation resulting in telomere dysfunction and increased genomic instability.

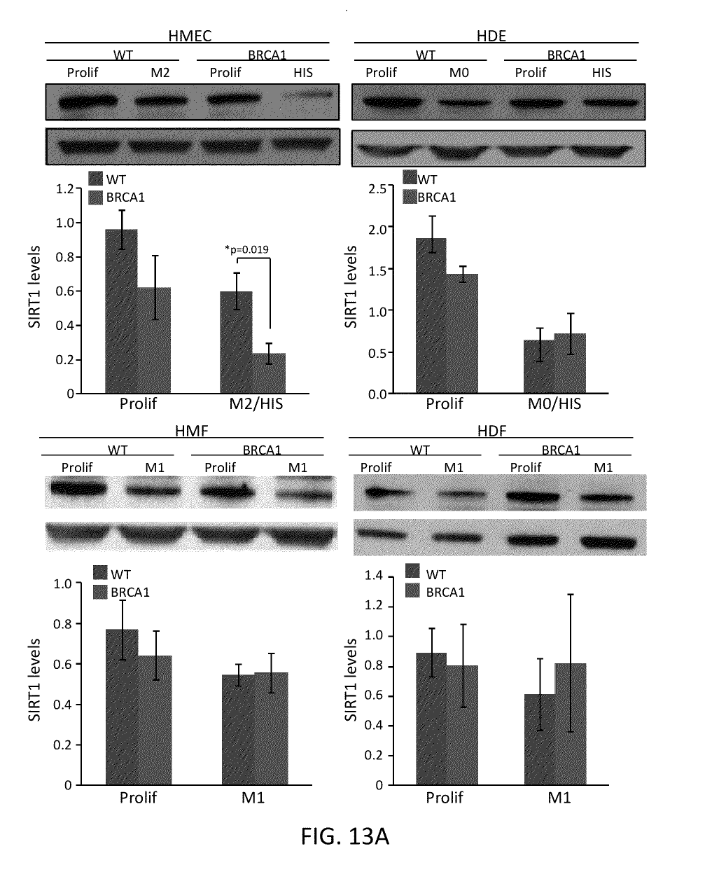

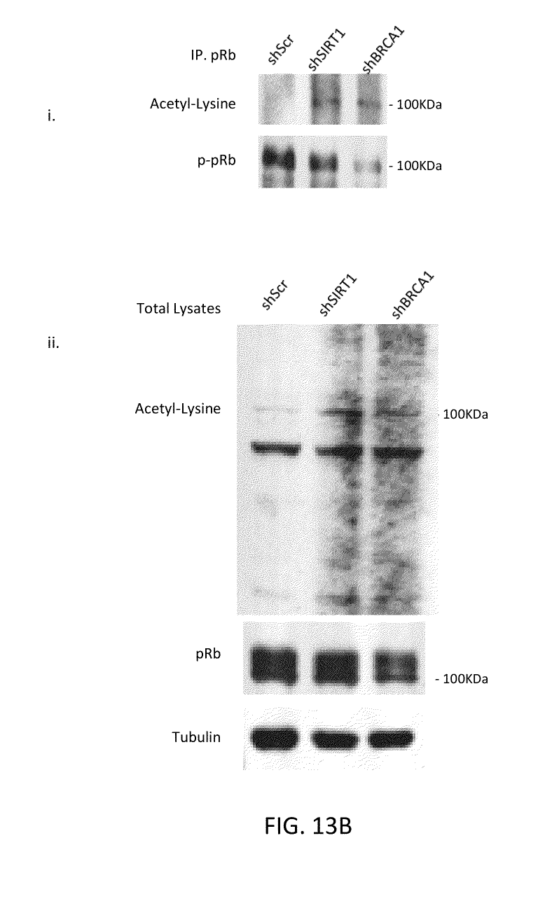

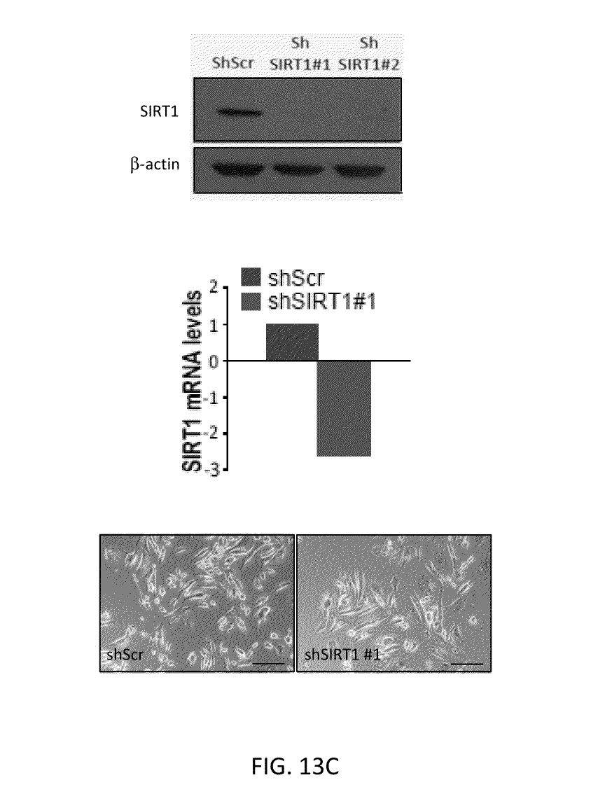

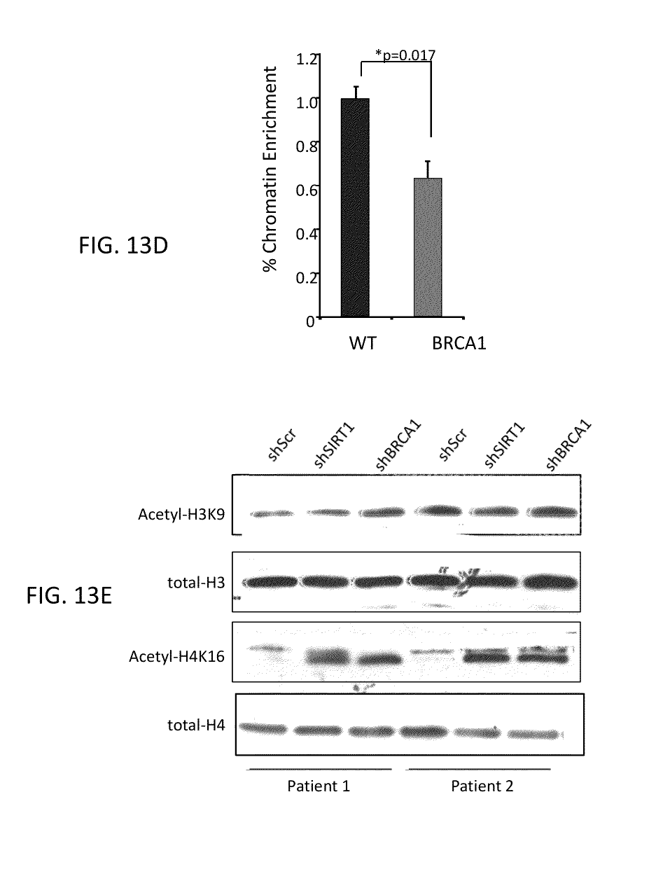

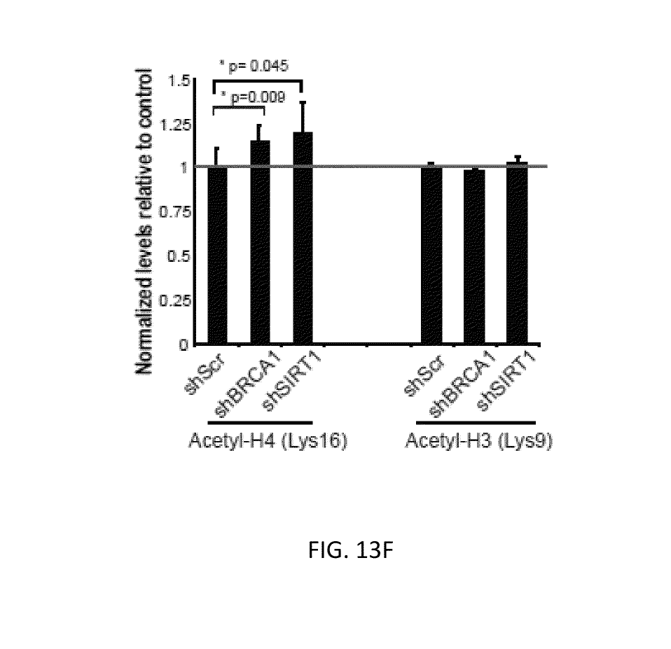

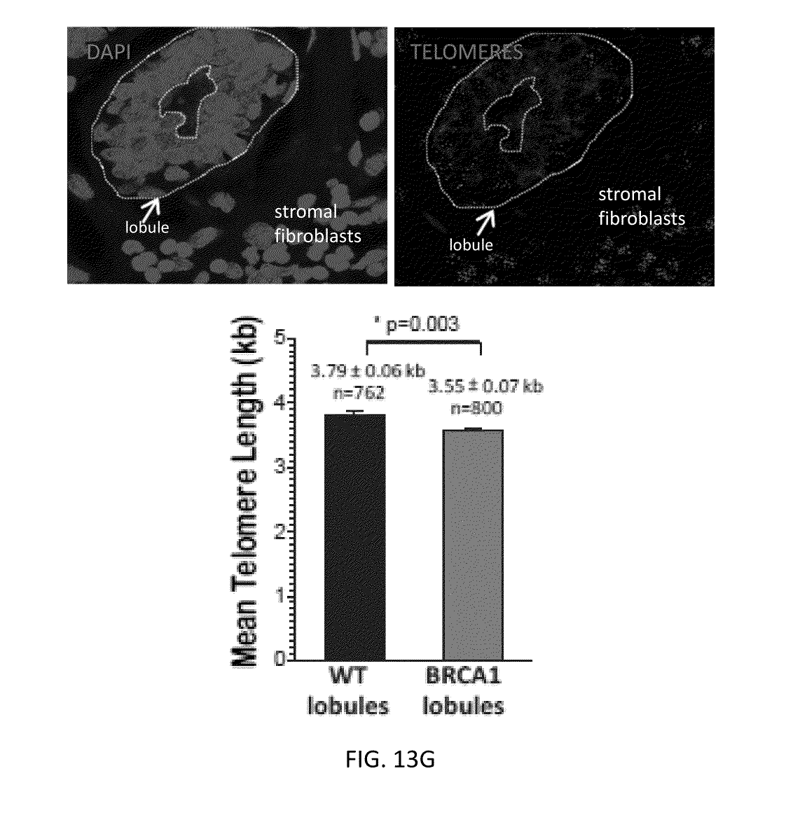

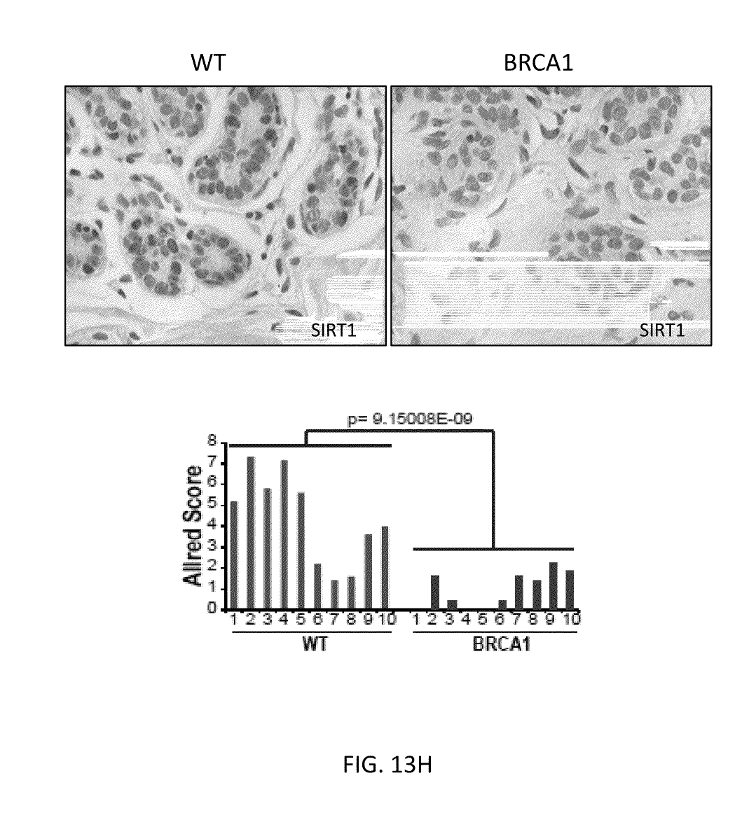

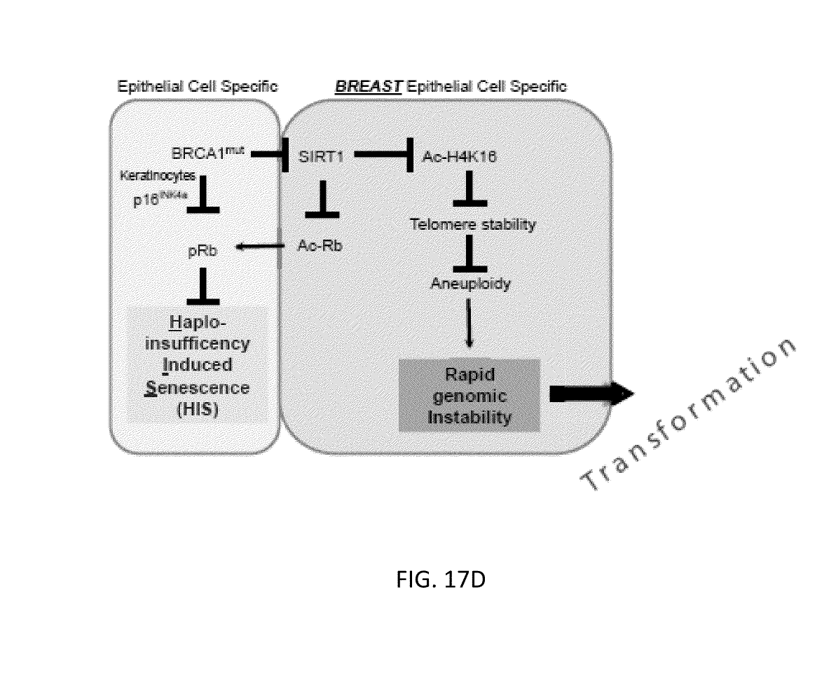

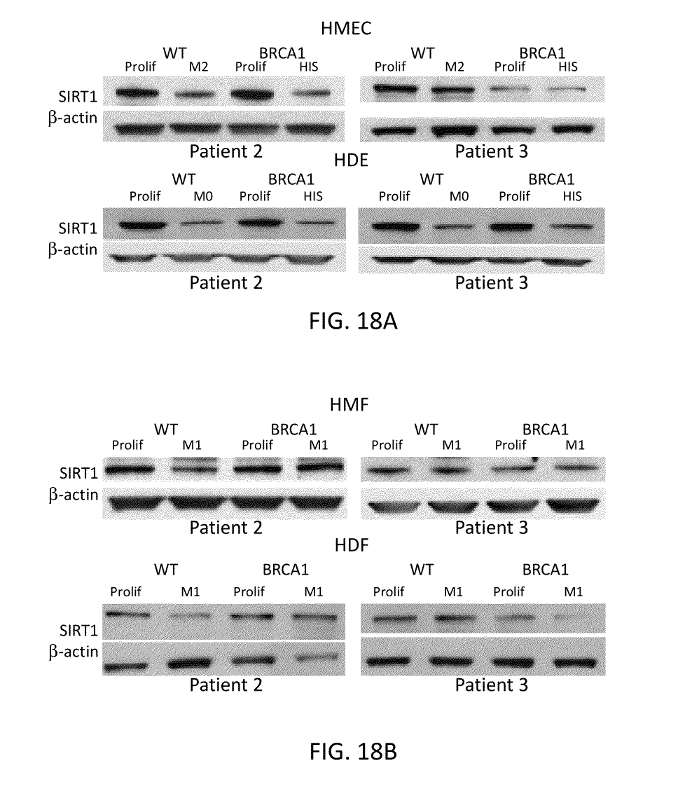

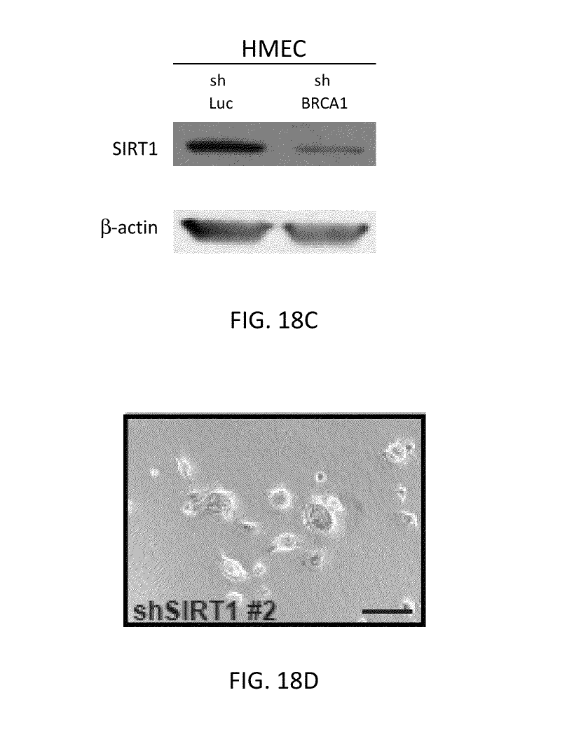

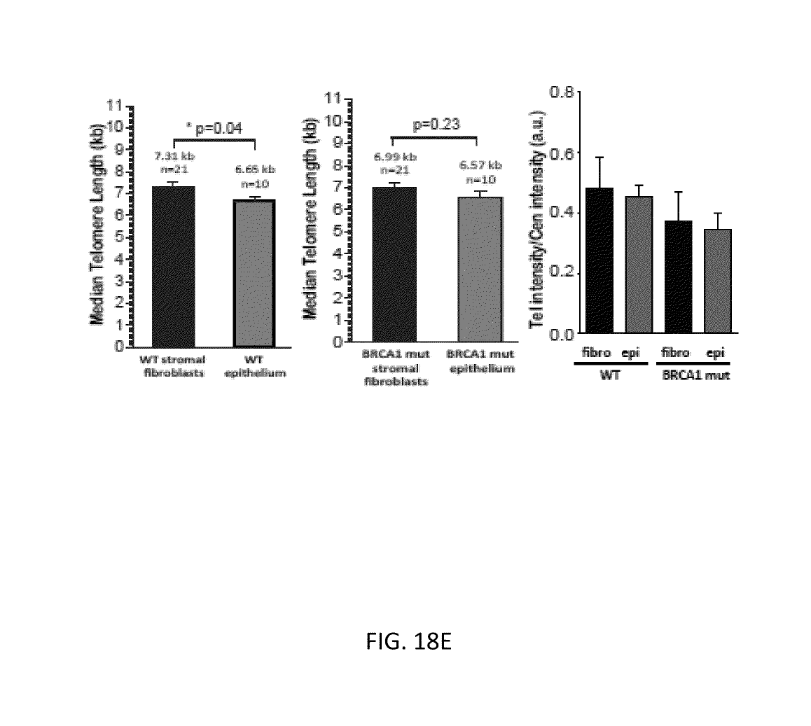

In support of this hypothesis, levels of SIRT1 in HMECs from BRCA1-mutation carriers were significantly reduced in senescent cells (p=0.019; FIGS. 13A and 18A). Further, SIRT1 levels were decreased in WT HMECs in which BRCA1 expression had been attenuated through lentiviral-mediated short hairpin inhibition (FIG. 18C), consistent with the notion that SIRT1 is a BRCA1 target. The decrease in SIRT1 was cell type-specific as the levels of SIRT1 in senescent BRCA1^{mut/+} HDEs, RMFs or HDFs did not differ from those found in senescent WT cells of the same tissue origin (FIGS. 13A, 18A and 18B). The decrease in SIRT1 expression was also associated with increased Ac-pRb (as well as increased acetylation of other proteins) in HMECs following knockdown of BRCA1 or SIRT1 (FIG. 13B). Furthermore, knockdown of SIRT1 in WT HMECs resulted in cell cycle arrest and morphological changes associated with senescence (FIGS. 13C and 18D). SIRT1 occupancy was also examined at telomeres and its levels were found to be significantly reduced in BRCA1^{mut/+} compared to WT HMECs (p=0.017; FIG. 13D). Histone substrates of SIRT1, specifically histone H4K16 acetylation, were also found to be altered in HMECs in which BRCA1 or SIRT1 was inhibited. Telomere-specific as well as global levels of H4K16Ac were markedly increased in either shBRCA1 or shSIRT1 HMECs, while no significant change in H3K9Ac was observed (FIGS. 13E, F). These findings demonstrate that haploinsufficiency of BRCA1 in HMECs, but not in other cell types, is associated with misregulation of SIRT1, leading to accumulation of H4K16-Ac and pRb-Ac, and thereby resulting in telomere erosion, genomic instability and pRb-dependent HIS.

Consistent with increased chromosomal abnormalities, unbalanced translocations, and the aneuploidy observed in BRCA1^{mut/+} HMECs in vitro (FIG. 10B), in vivo analysis of disease-free human breast tissues from BRCA1-mutation carriers has revealed similar genomic and chromosomal aberrations, as well as multipolar mitoses and abnormal centrosomes. To determine whether the other features of BRCA1 haploinsufficiency including telomere erosion, SIRT1 misregulation and HIS are also present in vivo, disease-free breast tissue specimens from BRCA1 mutation carriers were examined for telomere length, SIRT1 expression and evidence for pRb pathway activation. Telomere length was measured by Q-FISH with a telomeric probe in WT (N=21) and BRCA1^{mut/+} (N=9) tissues. Telomeres were indeed significantly shorter in breast epithelial cells within lobules of BRCA1^{mut/+} breast tissues compared to lobules of WT breast tissues (p=0.003, FIGS. 13G and 18E). This finding is of particular significance given that the cellular precursors to breast cancers reside within lobules. In addition, although breast epithelium from WT tissues was associated with overall shorter telomeres compared to breast stromal fibroblasts in WT tissues (FIG. 18E, p=0.04), this association was not observed in BRCA1-mutation carriers indicating that mechanisms regulating telomere length are altered in vivo.

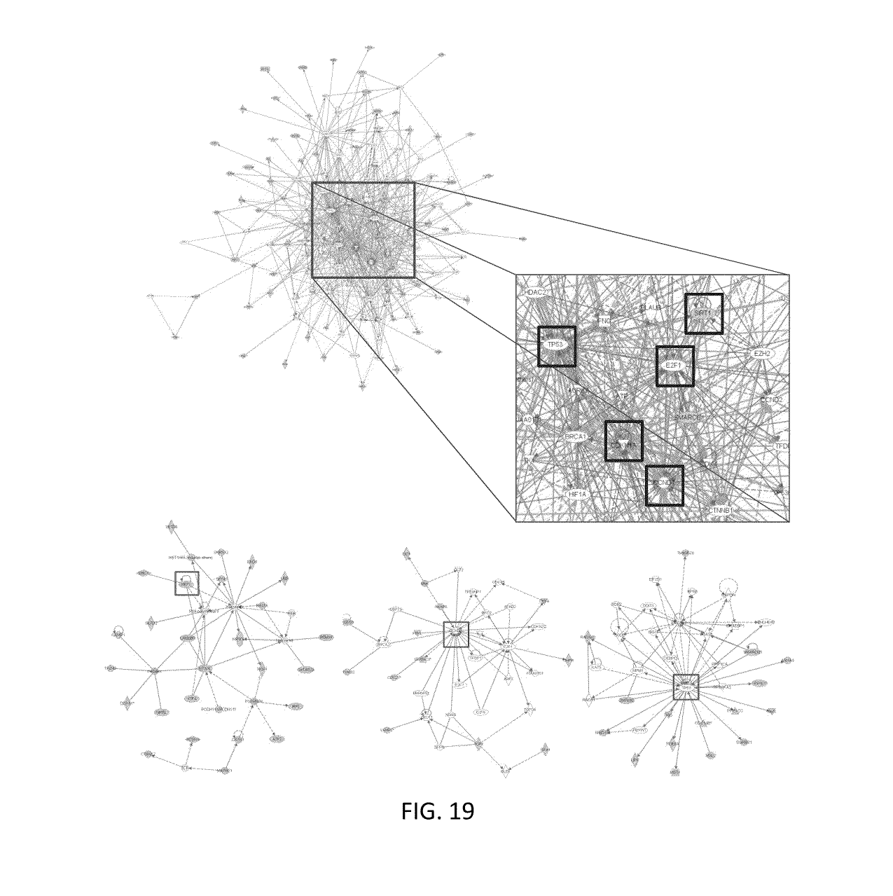

Likewise, SIRT1 expression and nuclear localization was significantly reduced in luminal cells within lobules of BRCA1^{mut/+} breast tissues compared to their WT counterparts (p=9.15×10⁻⁹; FIG. 13H), consistent with the lower levels of SIRT1 observed in vitro. Finally, gene expression data collected from freshly isolated breast epithelial cells from WT (N=4) and BRCA1-mutation carriers (N=4) was examined to determine whether evidence of DDR and HIS pathway activation could be observed in vivo. Consistent with in vitro findings, Ingenuity Pathway Analysis revealed ATM signaling (p=5.83×10⁻³), p53 signaling (p=4.51×10⁻¹), mismatch repair (p=3.44×10⁻¹), and cell cycle control of chromosomal replication (p=1.95×10⁻¹) as being significantly enriched in BRCA1^{mut/+} tissues. Moreover, comprehensive network analysis using Ingenuity Gene Network Analysis revealed 25 significant networks as major regulators in epithelial cells from BRCA1 mutation carriers, 12 of which formed an overlapping network with central nodes consisting of SIRT1, cyclin D1, CDKN1A and p53 (FIG. 19). Interestingly, additional networks involving cellular stress, metabolism, and autophagy were also enriched in vivo in BRCA1^{mut/+} tissues, consistent with the role of these pathways in regulating autophagy and senescence in response to DNA damage and chronic apoptotic stress (Brown, N. et al., Cancer Res., 72:6477-89, 2012; Salem, A. et al., Cell Cycle, 11:4167-73, 2012; Esteve, J. et al., Exp. Cell Res., 316:2618-29, 2010; Tang, M. & Wong, A., Cancer Res., 71(8, Suppl. 1):3790, 2011; Singh, K. et al., Autophagy, 8:236-51, 2012). Collectively, these findings indicate that breast epithelial cells in BRCA1 mutation carriers are poised for the rapid development of cancer due to misregulation of SIRT1, leading to telomere dysfunction, genomic instability, and pRB and p53 pathway activation.

A role for pRb in suppression of cellular replication and neoplastic transformation in BRCA1 haploinsufficient cells is supported by the high incidence of RB1 loss or mutations in human breast cancers with inactivated BRCA1 (Stefansson, O. et al., Epigenetics, 6:638-49, 2011; Hu, X. et al., Mol. Cancer. Res., 7:511-22, 2009; Jonsson, G. et al., Cancer Res., 72:4028-36, 2012). The findings described herein indicate that BRCA1^{mut/+} HMECs exhibit even greater genomic instability and telomeric fusions following forced proliferation beyond HIS indicates that a second non-proliferative barrier is triggered in response to excessive DDR and must be overcome for neoplastic transformation. Indeed, the increased genomic instability and elevated p53-dependent responses following pRb inhibition and in tissues from BRCA1-carriers is consistent with the role p53 likely plays upon loss of pRb during cancer progression. This is further supported by the observation that p53 is also frequently mutated or lost in BRCA1-associated breast cancers and in BRCA1-deficient murine cells that overcome senescence. Senescence is not a foolproof mechanism to prevent neoplastic transformation, as it has been shown to be bypassed following loss of p53 and pRb. These and other functionally related mutational events or hTERT re-expression overcome or bypass senescence, leading to rapid neoplastic transformation.

Haploinsufficiency for BRCA1 in disease-free breast epithelial cells from BRCA1-mutation carrier tissues also results in altered phenotypes and the misexpression of several genes involved in the establishment and/or maintenance of chromatin structure and concomitant defects in proper differentiation programs. Consistent with these findings, haploinsufficiency for BRCA1 in breast epithelial cells results in altered epigenetic histone modifications both globally and at telomeres. These findings demonstrate that a deleterious mutation in a single copy of BRCA1 is sufficient to induce a mutator phenotype driven by genetic and epigenetic events activating a novel form of senescence, which can be bypassed either through reactivation of hTERT or loss of pRb. Since mutation or loss of p53 and pRb pathways are obligate events in the pathogenesis of BRCA1-associated breast cancers, this previously unrecognized function of BRCA1 haploinsufficiency offers insights into the evolution of cancer in a tissue-specific manner associated with BRCA1-mutation carriers. Indeed, it is herein shown that of the cell and tissue types examined, only HMECs that are BRCA1 haploinsufficient lead to both pRb-dependent senescence and the means to overcome it through SIRT1-loss induced changes in telomere stability (FIG. 13I). Of note is the finding that ovarian epithelial cells from BRCA1-mutation carriers accumulate DNA double-strand breaks leading to premature ovarian aging (Titus, S. et al., Sci. Transl. Med., 5:172ra21, 2013).

### Cell Lines and Tissue Culture

All human breast tissue procurement for these experiments was obtained in compliance with the laws and institutional guidelines, as approved by the institutional IRB committee from Brigham and Women's Hospital and Tufts Medical Center. Disease-free prophylactic mastectomy (4 fresh, 10 formalin-fixed paraffin embedded) and skin tissue derived from women carrying a known deleterious BRCA1 heterozygous mutation were obtained with patient consent from the Surgical Pathology files or immediately following prophylactic mastectomy surgery. Tissues in which BRCA1 mutation was confirmed but not known were submitted for sequence/genotyping at Myriad Genetic Laboratories to confirm BRCA1 mutation. Non-BRCA1 tumor tissues were obtained from discarded material at Tufts Medical Center undergoing elective reduction mammoplasty at Tufts Medical Center. BRCA1 mutation status is listed in Table 1. The range of patient ages for fresh BRCA1^{+/+} tissue used in this study was 30-54 with a median age of 40; the range of patient ages for fresh BRCA1^{mut/+} tissue used in this study was 35-53 with a median age of 44. All disease-free breast tissues were verified by surgical pathologists prior to use in these studies.

HMECs were isolated and cultured in MEGM (Lonza) supplemented with bovine pituitary extract (BPE), insulin (5 μg/mL), EGF (10 ng/mL) and hydrocortisone (1 μg/mL). These cells were immortalized with the catalytic subunit of human telomerase (hTERT) (Elenbaas, B. et al., Genes Dev., 15:50-65, 2001). Human mammary fibroblasts (HMFs) were isolated and cultured in DMEM (Invitrogen) supplemented with 10% Calf Serum. Keratinocytes (HDEs) and dermal fibroblasts HDFs were isolated (Normand, J. & Karasek, M., In Vitro Cell Dev. Biol. Anim., 31:447-55, 1995). Briefly, skin tissue was chopped up into 0.5 cm cubes using a razor blade, and incubated overnight for digestion in a Dispase-containing solution. The following day, epidermis and dermis layers were separated and incubated in Collagenase-containing solution for 20 min at 37 C. Tissue/cell suspensions were pelleted, resuspended in trypsin, and frequently agitated to promote dissociation of cells. Dissociated epidermis layer was pelleted, plated and cultured in KGM-2 (Lonza) supplemented with bovine pituitary extract (BPE), insulin (5 μg/mL), hEGF (10 ng/mL), hydrocortisone (1 μg/mL), GA-1000 (gentamicin, amphotericin-B), Epinephrine and Transferrin. Dissociated dermis layer was pelleted, plated and cultured in DMEM (Invitrogen) supplemented with 10% Calf Serum.

### Lentiviral Constructs and Virus Production

The VSV-G-pseudotyped lentiviral vectors were generated by transient co-transfection of the vector construct with the VSV-G-expressing construct pCMV-VSVG and the packaging construct pCMV DR8.2Dvpr (Miyoshi, H. et al., J. Virol., 72:8150-7, 1998) into 293T cells together with FuGENE 6 transfection reagent (Roche). Lentiviral shRNA constructs targeting BRCA1, SIRT1 and pRb (Sigma Aldrich MISSION shRNA SHCLNG-NM_{—}007294, SHCLNG-NM_{—}012238 and SHCLNG-NM_{—}000321, respectively) were prepared (Gupta, P. et al., Nat. Genet., 37:1047-54, 2005).

### Western Blot Analysis

Cultured cells were harvested by trypsinization, pelleted and incubated in RIPA buffer supplemented with protease and phosphatase inhibitors (Roche) to obtain whole cell lysates. Cellular debris was removed by centrifugation at 13,000 rpm for 10 min. 30 μg of the whole cell lysate was used per sample. Western blotting was performed according to the manufacturer's protocol (BioRad). Briefly, 12% and/or 4-12% pre-cast gels (depending on the size of the proteins) and XT-MOPS running buffer were used for SDS-PAGE electrophoresis. 0.2 or 0.45 μm nitrocellulose membrane was used for protein transfer. Membranes were incubated overnight at 4 C with primary antibodies diluted in 1% bovine serum albumin in TBS-T. Secondary antibodies were applied for 1 hr at room temperature. The antibodies used included p16 (Santa Cruz), p53-Ser15 (Cell Signaling), p53-total (Santa Cruz), p21 (Santa Cruz), γH2AX (Cell Signaling), p27 (Santa Cruz), pRb-Ser795 (Cell Signaling), pRb-total (Santa Cruz), Cyclin E (Santa Cruz), Cyclin A (Santa Cruz), SIRT1 (Millipore), and β-actin (AbCam).

### Immunoprecipitation (IP)

shRNA-expressing WT HMECs (shScr, shBRCA1 and shSIRT1) were lysed in IP buffer (20 mM Tris pH 7.5, 150 mM NaCl, 1 mM EDTA, 1 mM EGTA, 1% Triton X-100) supplemented with protease and phosphatase inhibitors (Roche). For immunoprecipitation assays, protein lysates (200-600 μg) were combined with 2 μg of antibody and 25 μL of Protein A/G-Plus agarose beads (Santa Cruz, sc-2003). Following an overnight incubation at 4 C, agarose beads were extensively washed in IP buffer, resuspended in SDS sample buffer (125 mM Tris pH 6.8, 2.5% SDS, 10% glycerol, 2.5% 2-mercaptoethanol, 0.01% bromophenol blue) and loaded into a protein gel. Antibodies used in these experiments included anti-pRB (BD Pharmingen, #554136) and anti-Acetylated lysine (Cell Signaling, #9441).

### Histone Acid Extractions/Blots

Cells were harvested by trypsinization and acid extraction of histone proteins was carried out (Shechter, D. et al., Nat. Protoc., 2:1445-57, 2007). Briefly, cells were lysed in PBS with 0.5% Triton X-100, 2 mM PMSF and 0.02% NaN₃, nuclei were pelleted by centrifugation at 1000×g and the nuclear pellet was incubated at 4 C overnight in 0.2 N HCl. Western blotting was carried out as described above using 5 μg of acid-soluble lysate per sample. Antibodies used: anti-histone H3 (Cell Signaling #9715, 1:1000), anti-acetyl-histone H3K9 (Cell Signaling #9649P, 1:1000), anti-histone H4 (Millipore 07-108, 1:250), anti-acetylated histone H4K16 (Millipore 07-108, 1:500).

### Senescence Associated β-gal Assay

Senescence associated β-gal staining was performed according to the chromogenic assay as previously described (Debacq-Chainiaux, F. et al., Nat. Protoc., 4:1798-806, 2009). Briefly, cells were cultured in 6-well plates and fixed with formaldehyde/glutaraldehyde solution. After fixation, cells were washed twice in PBS. Samples were covered with staining solution and incubated overnight (12-16 h) at 37 C (no CO₂). Images were captured by bright filed microscopy.

### Quantitative RT-PCR

Total RNA from cultured cells was extracted with the RNeasy Mini Kit (QIAGEN). cDNA was prepared with an iScript kit (BioRad) and PCR was carried out with SYBR Green (BioRad). The following primers were used in this study:

Cyclin A:
(SEQ ID NO: 1)
Forward 5′-CGCTGGCGGTACTGAAGTC-3′
(SEQ ID NO: 2)
Reverse 5′-AAGGAGGAACGGTGACATGC-3′
IL-6:
(SEQ ID NO: 3)
Forward 5′-AACCTGAACCTTCCAAAGATGG-3′
(SEQ ID NO: 4)
Reverse 5′-TCTGGCTTGTTCCTCACTACT-3′
MMP2:
(SEQ ID NO: 5)
Forward 5′-CCGTCGCCCATCATCAAGTT-3′
(SEQ ID NO: 6)
Reverse 5′-CTGTCTGGGGCAGTCCAAAG-3′
IL-8:
(SEQ ID NO: 7)
Forward 5′-ACTGAGAGTGATTGAGAGTGGAC-3′
(SEQ ID NO: 8)
Reverse 5′-AACCCTCTGCACCCAGTTTTC-3′
PAI-1:
(SEQ ID NO: 9)
Forward 5′-GCTTGTCCAAGAGTGCATGGT-3′
(SEQ ID NO: 10)
Reverse 5′-AGGGCTGGTTCTCGATGGT-3′
Rb:
(SEQ ID NO: 11)
Forward 5′-GCCTCTCGTCAGGCTTGAG-3′
(SEQ ID NO: 12)
Reverse 5′-TCATCTAGGTCAACTCGTGCAA-3′
SIRT1:
(SEQ ID NO: 13)
Forward 5′-GCAGATTAGTAGGCGGCTTG-3′
(SEQ ID NO: 14)
Reverse 5′-GCTGGTGGAACAATTCCTGT-3′
p14:
(SEQ ID NO: 15)
Forward 5′-GGCCCTCGTGCTGATGCTAC-3′
(SEQ ID NO: 16)
Reverse 5′-TGGAGCAGCAGCAGCTCCGC-3′
p15:
(SEQ ID NO: 17)
Forward 5′-GGACTAGTGGAGAAGGTGCG-3′
(SEQ ID NO: 18)
Reverse 5′-GGGCGCTGCCCATCATCATG-3′
p16:
(SEQ ID NO: 19)
Forward 5′-CACCGAATAGTTACGGTCGG-3′
(SEQ ID NO: 20)
Reverse 5′-GCACGGGTCGGGTGAGAGTG-3′
p18:
(SEQ ID NO: 21)
Forward 5′-GGGGACCTAGAGCAACTTAC-3′
(SEQ ID NO: 22)
Reverse 5′-GTAGCAGTCTCCTGGCAATC-3′
p19:
(SEQ ID NO: 23)
Forward 5′-CTCAACCGCTTCGGCAAGAC-3′
(SEQ ID NO: 24)
Reverse 5′-GGACTGGTACCGGAGGTGTC-3′
GAPDH:
(SEQ ID NO: 25)
Forward 5′-GAGTCAACGGATTTGGTCGT-3′
(SEQ ID NO: 26)
Reverse 5′-TTGATTTTGGAGGGATCTCG-3′

GADPH was used as an internal control. Analysis was performed with the delta-delta Ct method.

### Immunofluorescence (IF)

Cells were cultured on 8-well chamber-slides and fixed with methanol at −20 C for 10 min. Samples were incubated overnight at 4 C with primary antibodies diluted in 1% bovine serum albumin PBS. Fluorescently labeled secondary antibodies were applied for 1 hr at room temperature. Cells were counterstained with DAPI. A Nikon Eclipse 80t microscope and SPOT camera were used for analyzing and photographing the stained sections. The antibodies used included Ki-67 (AbCam), γH2AX (Cell Signaling), p53BP (Cell Signaling), and pATM/ATR (Cell Signaling).

Telomere Chromatin Immunoprecipitation and qPCR

ChIP assays for shBRCA1 and shSIRT1 HMECs were performed as previously. In brief, after crosslink and sonication, chromatin from 4×10⁶ cells were used per each immunoprecipitation with protein A/G Plus agarose beads (Santa Cruz Biotechnology, sc-2003) and the following antibodies: 5 μg of anti-histone H3 (#ab1791, Abcam), 5 μg of anti-H3K9 (#H9286, Sigma), 5 μg anti-histone H4 (#ab10158, Abcam), 5 μg of anti-H4K16Ac (#39167, Active Motif) or pre-immune serum. The immunoprecipitated DNA was transferred to a Hybond N±membrane using a dot blot apparatus. The membrane was then hybridized with a telomeric probe containing TTAGGG repeats. Quantification of the signal was performed with ImageJ software. The amount of telomeric DNA after ChIP was normalized to the total telomeric DNA signal respectively for each genotype (input), as well as to the H3 and H4 abundance at these domains, thus correcting for differences in the number of telomere repeats or in nucleosome spacing.

ChIP on BRCA1^{mut/+} and WT HMECS were performed (Lee, T. et al., Nat. Protoc., 1:729-48, 2006), except that cross-linked nuclei were sonicated to 150-500 bp fragments in buffer containing 1% SDS, 50 mM Tris-HCl (pH 8.0), 10 mM EDTA, 1 mM PMSF, and complete protease inhibitors (Roche), and bound ChIP complexes were washed according to the Upstate/Millipore protocol, (Palacios, J. et al., J. Cell Biol., 191:1299-313, 2010; Mulligan, P. et al., Mol. Cell, 42:689-99, 2011). Antibodies used were: anti-SIRT1 (Cyclex Co. Ltd., Japan), anti-H4K16ac (Millipore, Mass., USA) and anti-histone H3 (Abcam, UK). Quantitative PCR analysis of telomeric sequences was performed using forward primer (5′-CGGTT TGTTT GGGTT TGGGT TTGGG TTTGG GTTTG GGTT; SEQ ID NO:27) and reverse primer (5′-GGCTT GCCTT ACCCT TACCC TTACC CTTAC COTTA CCC; SEQ ID NO:28) at an annealing temperature of 60 C.

### Immunohistochemistry (1HC)

Immunohistochemistry was performed on formalin-fixed, paraffin-embedded tissue sections with sodium citrate antigen retrieval, followed by visualization with the ABC Elite peroxidase kit and DAB substrate (Vector Labs) for detection of SIRT1 (Millipore). IHC results were semi-quantitatively analyzed using the Allred Score.

### Chromosomal Metaphase Analysis

Cultures were checked for harvest on the third day after trypsinization, and 30 μL of colcemid (10 μg/mL Gibco) was added per 5 mL of culture medium. Cultures were incubated for 30 mins at 37 C. Cells were detached from flasks with trypsin and the supernatant and cells were spun at 1,100 rpm for 5 mins. The supernatant was discarded and replaced with 2:1 hypotonic solution (2 parts 0.075 M potassium chloride to one part 0.6% sodium citrate). The cultures were incubated at 37 C for 20 mins, and then fixed with several changes of fixative (methanol, acetic acid). Slides were prepared, treated with trypsin and stained with Wright's-Giemsa.

### Telomere Length Assays

The overall telomere lengths for each experimental sample were determined relative to the reference DNA by comparing the difference in their ratios of the telomere copy number (T) to the single copy gene copy number (S) using quantitative PCR. This ratio is proportional to the mean telomere length (Cawthon, R., Nucl. Acids Res., 37:e21, 2009). A modified qPCR assay was used for telomere sequence quantitation that is compatible with Applied Biosystems 7900 HT instrumentation. Each plate (384 wells on each plate) contained a set of standards spanning an 81-fold range prepared by serial dilution, and each sample was analyzed in triplicate. Two master mixes of PCR reagents were prepared, one with the telomere primers (telc and telg) and the other with either the albumin pair (albd, and albu) or the beta-globin pair (hgbu, and hgbd). The final concentrations in each PCR reaction were 0.8×SYBR Green I Master Mix (Agilent Technologies), and 900 nM of the telomere pair, 900 nM of the albumin pair or 500 nM of the beta-globin pair. The thermal cycling profile used was 15 min at 95 C, 2 cycles of 15s at 94 C, 15s at 49 C, followed by 32 cycles of 15s at 94 C, 10s at 62 C, and 15s at 74 C with data acquisition. The plates were read at 74 C to minimize the interference from the telomere primer-dimers. The ABI software SDS version 2.0 was used to generate two standard curves from each plate, one for the telomere amplification, and the other for the single copy gene. The ratio (T/S) of the telomere copy number (T) to the single gene copy number (S) was generated for each experimental sample, and the value averaged across the triplicates, which provides the average telomere length for each experimental sample. The T/S ratios relative to the reference sample were generated using the comparative CT (cycle threshold) method.

### Allele Specific Loss of Heterozygosity Studies

PCR primers were designed flanking the BRCA1 mutations from the individuals in the study (187delAG, 2800delAA, 4184del4, 5385insC, 943ins10 and 4154delA). PCR products were treated with ExoSap-It (USB) and sequenced. Sequence traces in the forward and reverse direction were compared between control blood DNAs of individuals with these germline mutations and the different derivatives of primary human mammary epithelial cells from individuals with these mutations using DNAstar 3.0. Loss was determined visually by two reviewers and consisted of at least 30% difference between the two alleles compared to normal carrier ratios as described (Spearman, A. et al., J. Clin. Oncol., 26:5393-400, 2008).

### Quantitative Telomere Fluorescence In Situ Hybridization

For qFISH analysis on breast tissue samples, de-paraffinated sections were hybridized with a PNA-tel Cy3-labeled probe, and telomere length was determined as described (Zijlmans, J. et al., Proc. Natl. Acad. Sci. USA, 94:7423-8, 1997; Gonzalez-Suarez, E. et al., Nat. Genet., 26:114-7, 2000; Samper, E. et al., EMBO Rep., 1:244-52, 2000; Munoz, P. et al., Nat. Genet., 37:1063-71, 2005; Flores, I. et al., Genes Dev., 22:654-67, 2008). DAPI and Cy3 signals were acquired simultaneously into separate channels using a confocal ultraspectral microscope Leica TCS-SP5 and maximum projections from image stacks were generated for image quantification.

For image acquisition we used a new tool for intelligent screening named “matrix screening remote control (MSRC)” developed at CNIO. The MSRC application manages a first fast scan with low-resolution settings, generating one image per sample of the whole tissue and later localizes the areas of interest, extracting their coordinates and surface area. With the spatial information, the MSRC application interacts with the microscope and load high-resolution settings, scanning automatically just the areas of interest.

Quantitative image analysis of telomere fluorescence intensity was performed on confocal images using the Definiens Developer Cell software (Definiens Developer XD). The DAPI image was used to define the nuclear areas that were separated by a Cellenger-Solution. After defining the nuclear areas a predefined Ruleset was used for the quantification of telomere fluorescence intensity (Cy3 image). The fluorescence values for each section were exported to GraphPad Prism, and graphs were generated. The total number of telomeric spots scored for each genotype is shown. Student's t-test was used for statistical analysis.

### BRCA1^{Mut/+} Gene Expression Analysis, GSEA and Network Analysis

Gene Set Enrichment Analysis (GSEA) was applied to previously published gene expression data collected on cultured proliferating primary human mammary epithelial cells isolated from BRCA1-mutation carriers (N=6) or age-matched WT (N=6) (GSE19383, Bellacosa, A. et al., Cancer Prev. Res. (Phila.), 3:48-61, 2010). Two-sided T-tests were run on the gene sets and the top 2000 genes from each set were ranked. Gene Set Enrichment Analysis (GSEA) was performed (Subramanian, A. et al., Proc. Natl. Acad. Sci. USA, 102:15545-50, 2005). Gene networks were constructed from gene expression data collected on freshly isolated human mammary epithelial cells isolated from BRCA1-mutation carriers (N=4) or age-matched WT (N=4) (GSE25835). Important hubs were identified using Ingenuity Pathway Analysis (IPA; Ingenuity Systems, Mountain View, Calif.) based on differentially expressed genes between BRCA1^{mut/+} and WT patients (n=701 genes).

### Other Embodiments

Other embodiments will be evident to those of skill in the art. It should be understood that the foregoing detailed description is provided for clarity only and is merely exemplary. The spirit and scope of the present disclosure are not limited to the above examples, but are encompassed by the following claims and their equivalents. The contents of all references cited herein are incorporated by reference in their entireties.

What is claimed is:

1. A method for prophylactically treating an individual at risk for breast cancer or ovarian cancer, comprising identifying the individual as comprising a genotype that comprises one copy of a defective BRCA1 allele and one copy of a functional BRCA1 allele, and administering a prophylactically effective amount of a SIRT1 agonist to the individual.

2. The method of claim 1, wherein the prophylactic treatment prevents or ameliorates a pre-malignant condition associated with breast cancer or ovarian cancer.

3. The method of claim 1, wherein the SIRT1 agonist is selected from the group consisting of: a small molecule agonist, an activating antibody and an enzymatic agonist.

4. The method of claim 5, wherein the SIRT1 agonist is selected from the group consisting of: butein, fisetin, isonicotinamide, piceatannol, quercetin and resveratrol.

5. A method for prophylactically treating an individual at risk for breast cancer or ovarian cancer, comprising identifying the individual as comprising a genotype that comprises one copy of a defective BRCA1 allele and one copy of a functional BRCA1 allele, and administering a prophylactically effective amount of a deacetylase that deacetylates Rb.

6. The method of claim 5, further comprising administering a prophylactically effective amount of a Rb phosphorylase.
